(19) United States
(12) Patent Application Publication
(10) Pub. No.: US 2012/0130432 A1
(43) Pub. Date: May 24, 2012

(54) Methods and Apparatus for Treating Spinal Stenosis

(75) Inventors: Bret A. Ferree, Cincinnati, OH (US); David Tompkins, Milford, OH (US)

(73) Assignee: NUVASIVE, INC., San Diego, CA (US)

(21) Appl. No.: 13/299,328

(22) Filed: Nov. 17, 2011

Related U.S. Application Data
(63) Continuation of application No. 11/051,692, filed on Feb. 4, 2005, now abandoned.
(60) Provisional application No. 60/629,018, filed on Nov. 18, 2004.

(51) Int. Cl. A61B 17/88

(52) U.S. Cl. 606/279

(57) ABSTRACT

Surgical implants are configured for placement posteriorly to a spinal canal between vertebral bodies to distract the spine and enlarge the spinal canal. The device permits spinal flexion while limiting spinal extension thereby providing an effective treatment for treating spinal stenosis without the need for laminectomy. The device may be used in the cervical, thoracic, or lumbar spine. Numerous embodiments are disclosed, including elongated, length-adjustable components coupled to adjacent vertebral bodies using pedicle screws. The device is configured for placement between adjacent vertebral bodies and adapted to fuse the lamina, facet, spinous process or other posterior elements of a single vertebra. Preferably, the device forms a pseudo-joint in conjunction with the non-fused vertebra. Alternatively, the device could be fused to the caudal vertebra or both the cranial and caudal vertebrae.

### REFERENCE TO RELATED APPLICATION

This application claims priority from U.S. Provisional Application Ser. No. 60/629,018, filed Nov. 18, 2004, the entire content of which is incorporated herein by reference.

### FIELD OF THE INVENTION

This invention relates generally to spine surgery and, in particular, to methods and apparatus for treating spinal stenosis.

### BACKGROUND OF THE INVENTION

Spinal stenosis is a narrowing of spaces in the spine, results in pressure on the spinal cord and/or nerve roots. This disorder usually involves the narrowing of one or more of the following: (1) the canal in the center of the vertebral column through which the spinal cord and nerve roots run, (2) the canals at the base or roots of nerves branching out from the spinal cord, or (3) the openings between vertebrae through which nerves leave the spine and go to other parts of the body.

Pressure on the lower part of the spinal cord, or on nerve roots branching out from that area, may give rise to pain or numbness in the legs. Pressure on the upper part of the spinal cord (that is, the neck area) may produce similar symptoms in the shoulders, or even the legs. The condition generally occurs in patients who are in their last decade or decades of life.

Laminectomy, which involves removing bone, the lamina, from the vertebrae, is the most common surgical treatment for spinal stenosis. Laminectomy enlarges the spinal canal, thus relieving the pressure on compressed nerves. Surgical burs, drills, punches, and chisels are used during the procedure.

Surgeons risk injuring the nerves or the spinal cord as they enlarge the spinal canal. In addition, elderly patients frequently have co-morbidities that increase the risk of laminectomy. Complications of laminectomy include increased back pain, infection, nerve injury, blood clots, paralysis, prolonged recovery, and death.

Lumbar fusion is frequently preformed in-conjunction with laminectomy. Current fusion techniques require abrasion of large surfaces of bone. Bone bleeds during and after abrasion. Current fusion techniques increase the risk of spinal stenosis procedures.

Fusion also prolongs patient recovery following spinal stenosis surgery. Patients and surgeons would welcome less invasive treatments for spinal stenosis.

### BRIEF DESCRIPTION OF THE DRAWINGS

FIG. 1A is a lateral view of a novel device of the invention;

FIG. 1B is a lateral view of the embodiment of the invention shown in FIG. 1A;

FIG. 1C is an end view of a novel vertebral screw and a cross-section of the threaded portion of the rod shown in FIG. 1A;

FIG. 1D is an end view of the screw shown in FIG. 1C and a cross-section of the threaded end component shown in FIG. 1A;

FIG. 1E is an end view of an alternative embodiment of the screw shown in FIG. 1C;

FIG. 1F is a lateral view of the device shown in FIG. 1A and the screws of the embodiment shown in FIG. 1C;

FIG. 1G is a lateral view of the embodiment of the device shown in FIG. 1F;

FIG. 1H is a lateral view of the device shown in FIG. 1G;

FIG. 1I is a sagittal cross-section of the device shown in FIG. 1H;

FIG. 2A is a lateral view of an alternative embodiment of the invention;

FIG. 2B is an exploded view of the device shown in FIG. 2A and an alternative embodiment of the vertebral screws;

FIG. 2C is a lateral view of the device shown in FIG. 2B;

FIG. 2D is an exploded end view of the screw and fastening component shown in FIG. 2C;

FIG. 2E is end view of the screw and fastening component shown in FIG. 2D and a cross-section of the rod-like device shown in FIG. 2C;

FIG. 3A is a lateral view of an alternative embodiment of the device shown in FIG. 2A;

FIG. 3B is a lateral view of the embodiment of the device shown in FIG. 3A;

FIG. 3C is a view of the top of the device shown in FIG. 3B;

FIG. 3D is an enlarged view of the top of the hinge joint shown in FIG. 3C;

FIG. 3E is an enlarged view of the top of an alternative embodiment of the hinge joint shown in FIG. 3D;

FIG. 3F is a lateral view of the device shown in FIG. 3B and vertebral screws;

FIG. 4A is an exploded lateral view of an alternative embodiment of a vertebral screw and a portion of the device shown in FIG. 3B;

FIG. 4B is a lateral view of the embodiment of the device shown in FIG. 4A;

FIG. 4C is a lateral view of the embodiment of the device shown in FIG. 4B;

FIG. 5A is a lateral view of an alternative embodiment of the device shown in FIG. 2A;

FIG. 5B is a lateral view of the device shown in FIG. 5A. The device is shown in its contracted position;

FIG. 5C is an exploded view of the device shown in FIG. 5A and vertebral screws;

FIG. 5D is a lateral view of the assembled device shown in FIG. 5C;

FIG. 6A is an oblique view of an alternative embodiment of the invention;

FIG. 6B is a lateral view of a portion of the spine;

FIG. 6C is a lateral view of a portion of the spine and the embodiment of the invention shown in FIG. 6A;

FIG. 6D is a lateral view of a portion of the spine and the device shown in FIG. 6A;

FIG. 6E is an oblique view of a cancellous bone block;

FIG. 6F is an oblique view of a section of the shaft of a long bone;

FIG. 6G is an oblique view of a section of the shaft of a long bone and a cancellous bone block;

FIG. 6H is an oblique view of the embodiment of the invention shown in FIG. 6G;

FIG. 6I is lateral view of a portion of the spine and a sagittal cross-section of the embodiment of the device shown in FIG. 6A;

FIG. 7A is an oblique view of a portion of a shaft of a long bone;

FIG. 7B is an oblique view of portion of a shaft of a shaped long bone;

FIG. 7C is a lateral view of the spine and the embodiment of the invention shown in FIG. 7B;

FIG. 7D is an oblique view of a piece of bone;

FIG. 7E is an end view of the device shown in FIG. 7A and the bone shown in FIG. 7D;

FIG. 7F is a lateral view of a portion of the spine and a sagittal cross-section of the embodiment of the device shown in FIG. 7E;

FIG. 8A is an end view of an alternative shape of the device shown in FIG. 7B;

FIG. 8B is an end view of an alternative shape of the device shown in FIG. 8A;

FIG. 8C is an end view of an alternative shape of the device shown in FIG. 8B;

FIG. 9A is an oblique view of an alternative embodiment of the device shown in FIG. 6A;

FIG. 9B is a view of the top of the embodiment of the invention shown in FIG. 8A;

FIG. 9C is a lateral view of a portion of the spine and the embodiment of the invention shown in FIG. 9A;

FIG. 10A is an oblique drawing of an alternative embodiment of the invention related to that shown in FIG. 6A;

FIG. 10B is a lateral view of a portion of the spine and the embodiment of the invention shown in FIG. 10A;

FIG. 10C is a dorsal view of a portion of the spine and the embodiment of the invention shown in FIG. 10A;

FIG. 10D is a sagittal cross-section of the embodiment of the invention shown in FIG. 10B and a lateral view of the spine;

FIG. 10E is a sagittal cross-section of the embodiment of the invention shown in FIG. 10D and a lateral view of the spine;

FIG. 10F is a coronal cross section of the embodiment of the invention shown in FIG. 10E and the spine;

FIG. 11A is a dorsal view of the embodiment of the invention shown in FIG. 10F and the spine;

FIG. 11B is a dorsal view of the embodiment of the invention shown in FIG. 11A and the spine;

FIG. 12A is a lateral view of the spine and the embodiment of the invention shown in FIG. 11A;

FIG. 12B is a dorsal view of the embodiment of the invention shown in FIG. 12A and the spine;

FIG. 12C is a lateral view of the spine and the embodiment of the invention shown in FIG. 12A;

FIG. 12D is a lateral view of, the spine and the embodiment of the invention shown in FIG. 12C;

FIG. 13A is a lateral view of the spine and the embodiment of the invention shown in FIG. 10B;

FIG. 13B is a dorsal view of the embodiment of the invention shown in FIG. 13A and the spine;

FIG. 14A is a lateral view of the spine and an alternative embodiment of the invention shown in FIG. 13A;

FIG. 14B is a dorsal view of the embodiment of the invention shown in FIG. 14A and the spine;

FIG. 14C is a dorsal view of the spine and the embodiment of the invention shown in FIG. 14B;

FIG. 14D is a dorsal view of the spine and an alternative embodiment of the invention shown in FIG. 14C;

FIG. 15 is a lateral view of the spine and the embodiment of the invention shown in FIG. 10A;

FIG. 16 is a dorsal view of the spine and the embodiment of the invention shown in FIG. 10A;

FIG. 17 is a dorsal view of the spine and the embodiment of the invention shown in FIG. 10A;

FIG. 18 is a lateral view of the spine and the embodiment of the invention shown in FIG. 10A;

FIG. 19 is a lateral view of the spine and the embodiment of the invention shown in FIG. 10A;

FIG. 20A is a dorsal view of the spine and an alternative embodiment of a device related to that shown in FIG. 17;

FIG. 20B is a lateral view of the spine and the embodiment of the invention shown in FIG. 20A;

FIG. 21A is a dorsal view of the spine and an alternative embodiment of the invention related to that shown in FIG. 20A;

FIG. 21B is a dorsal view of the spine and the embodiment of the invention shown in FIG. 21A;

FIG. 22 is a lateral view of the spine and an alternative embodiment of the invention related to that shown in FIG. 12A;

FIG. 23A is a lateral view of the spine and an alternative embodiment of the invention shown in FIG. 22;

FIG. 23B is a dorsal view of the spine and the embodiment of the invention shown in FIG. 23A;

FIG. 24A is an exploded dorsal view of the spine and an alternative embodiment of the invention related to that shown in FIG. 23B;

FIG. 24B is a dorsal view of the spine and the embodiment of the invention shown in FIG. 24A;

FIG. 25A is a lateral view of the spine and an alternative embodiment of the invention related to that shown in FIG. 12A;

FIG. 25B is a dorsal view of the spine and the embodiment of the invention shown in FIG. 25A;

FIG. 26A is a lateral view of the spine and an alternative embodiment of the invention related to that shown in FIG. 25A;

FIG. 26B is dorsal view of the spine and the embodiment of the invention shown in FIG. 26A;

FIG. 26C is a sagittal cross-section of an alternative embodiment of the invention related to that shown in FIG. 26B;

FIG. 27A is a lateral view of an alternative embodiment of the device related to that shown in FIG. 26A;

FIG. 27B is a dorsal view of the spine and the embodiment of the invention shown in FIG. 27A;

FIG. 28A is a lateral view of the spine and the embodiment of the invention shown in FIG. 10A;

FIG. 28B is a lateral view of the spine and the embodiment of the invention shown in FIG. 28A;

FIG. 28C is a lateral view of the spine and the embodiment of the invention shown in FIG. 28B;

FIG. 29 is a lateral view of the spine, the embodiment of the invention shown in FIG. 12A, and a device to help prevent extrusion of the spinous process spacer;

FIG. 30 is a view of the caudal aspect of the cranial vertebra shown in FIG. 28C;

FIG. 31 shows a dorsal view of the spine;

FIG. 32A is a lateral view of the spine and the embodiment of the invention shown in FIG. 12A;

FIG. 32B is a lateral view of the spine and an alternative embodiment of the invention related to that shown in FIG. 32A;

FIG. 32C is a lateral view of the spine and an alternative multilevel embodiment of the invention related to that shown in FIG. 32B;

FIG. 33 is a lateral view of the spine and an alternative multilevel embodiment of the invention related to that shown in FIG. 32C;

FIG. 34 is a lateral view of the spine and an alternative embodiment of the invention related to that shown in FIG. 12A;

FIG. 35A is a lateral view of the spine and an alternative multilevel embodiment of the invention related to that shown in FIG. 20B;

FIG. 35B is a dorsal view of the spine and the embodiment of the invention shown in FIG. 35A;

FIG. 35C is a dorsal view of an alternative embodiment of the invention related to that shown in FIG. 20B;

FIG. 35D is a dorsal view of the spine and a three-level version of the device shown in FIG. 35B;

FIG. 36A is a dorsal exploded view of the spine and an alternative embodiment of the device related to that shown in FIG. 35B;

FIG. 36B is dorsal view of the spine and the embodiment of the invention shown in FIG. 36A;

FIG. 37 is a lateral view of the spine and an alternative embodiment of the invention related to that shown in FIG. 33;

FIG. 38A is an exploded lateral view of spine and an alternative embodiment of the invention related to that shown in FIG. 12A;

FIG. 38B is a lateral view of the spine and the embodiment of the invention shown in FIG. 38A;

FIG. 38C is a lateral view of the spine and an alternative embodiment of the invention related to that shown in FIG. 38B;

FIG. 39A is a dorsal view of an alternative embodiment of the invention related to that shown in FIG. 38C;

FIG. 39B is a dorsal view of the embodiment of the invention shown in FIG. 39A;

FIG. 40A is a dorsal view of an alternative embodiment of the device related to that shown in FIG. 39A;

FIG. 40B is a dorsal view of the embodiment of the invention shown in FIG. 40A;

FIG. 40C is a lateral view of the spine and the embodiment of the device shown in FIG. 40B;

FIG. 41A is an exploded oblique view of an alternative embodiment of the invention related to that shown in FIG. 10A;

FIG. 41B is a lateral view of the embodiment of the device shown in FIG. 41A;

FIG. 41C is a dorsal view of the embodiment of the invention shown in FIG. 41B;

FIG. 42 is an oblique view of an alternative embodiment of the invention related to that shown in FIG. 41C;

FIG. 43A is an exploded oblique view of an alternative embodiment of the invention related to that shown in FIG. 41A;

FIG. 43B is an oblique view of the device shown in FIG. 43B;

FIG. 44A is an exploded oblique view of an alternative embodiment of the invention related to that shown in FIG. 43A;

FIG. 44B is an oblique view of the invention shown in FIG. 44A;

FIG. 45 is an oblique view of an alternative embodiment of the invention related to that shown in FIG. 44B;

FIG. 46 is an oblique view of an alternative embodiment of the invention related to that shown in FIG. 45;

FIG. 47A is an exploded oblique view of an alternative embodiment of the invention related to that shown in FIG. 46;

FIG. 47B is an oblique view of the embodiment of the invention shown in FIG. 47A;

FIG. 48A is an exploded oblique view of an alternative embodiment of the invention related to that shown in FIG. 47A;

FIG. 48B is an oblique view of the embodiment of the invention shown in FIG. 48A;

FIG. 49A is lateral view of an alternative embodiment of the invention related to that shown in FIG. 10A;

FIG. 49B is an exploded lateral view of the embodiment of the device shown in FIG. 49A;

FIG. 49C is a lateral view of an alternative embodiment of the invention related to that shown in FIG. 49A;

FIG. 50A is lateral view of an alternative embodiment of the device related to that shown in FIG. 49C;

FIG. 50B is a dorsal view of the embodiment of the invention shown in FIG. 50A;

FIG. 50C is an oblique view of bones shaped to be connected in an alternative method according to the invention;

FIG. 50D is a lateral view of an alternative embodiment of the device related to that shown in FIG. 49C;

FIG. 51A is an oblique view of an alternative embodiment of the invention related to that shown in FIG. 10A;

FIG. 51B is a dorsal view of the spine and the embodiment of the invention shown in FIG. 51A;

FIG. 51C is a caudal view of the embodiment of the device shown in FIG. 51A;

FIG. 52 is dorsal view of the spine and an alternative embodiment of the invention related to that shown in FIG. 51B;

FIG. 53 is a dorsal view of the spine and an alternative embodiment of the invention related to that shown in FIG. 52;

FIG. 54A is a lateral view of the spine and an alternative embodiment of the invention related to that shown in FIG. 53;

FIG. 54B is a dorsal view of the spine and the device shown in FIG. 54A;

FIG. 55 is a dorsal view of the spine and an alternative embodiment of the device shown in FIG. 54B;

FIG. 56A is a dorsal view of the spine and an alternative embodiment of the invention related to that shown in FIG. 10A;

FIG. 56B is a lateral view of the spine and the embodiment of the invention shown in FIG. 56A;

FIG. 57 is a dorsal view of the spine and an alternative embodiment of the invention related to that shown in FIG. 56A;

FIG. 58 is a lateral view of the spine and an alternative embodiment of the invention related to that shown in FIG. 10A;

FIG. 59 is a lateral view of the spine and the embodiment of the invention shown in FIG. 10A;

FIG. 60A is a lateral view of the spine and an exploded lateral view of an alternative embodiment of the invention related to that shown in FIG. 10A;

FIG. 60B is a lateral view of the spine and the embodiment of the invention shown in FIG. 60A;

FIG. 61 is a lateral view of the spine and an alternative embodiment of the invention related to that shown in FIG. 10A;

FIG. 62A is a dorsal view of the spine and an alternative embodiment of the invention related to that shown in FIG. 10C;

FIG. 62B is a dorsal view of the spine and the embodiment of the invention shown in FIG. 62A;

FIG. 63A is a lateral view of the spine and an alternative embodiment of the invention related to that shown in FIG. 62A;

FIG. 63B is a lateral view of the spine and the embodiment of the device shown in FIG. 63A;

FIG. 64A is a lateral view of the spine and an alternative embodiment of the invention related to that shown in FIG. 63B;

FIG. 64B is a lateral view of the spine and the embodiment of the invention shown in FIG. 64A;

FIG. 65A is a dorsal view of an alternative embodiment of the invention related to that shown in FIG. 10A;

FIG. 65B is a dorsal view of the embodiment of the device shown in FIG. 65A;

FIG. 66A is an exploded oblique view of an alternative embodiment of the invention related to that shown in FIG. 41A;

FIG. 66B is an oblique view of the device shown in FIG. 66A;

FIG. 67 is a dorsal view of the spine and an alternative embodiment of the invention related to that shown in FIG. 10A;

FIG. 68 is a lateral view of the spine and an alternative embodiment of the device related to that shown in FIG. 12A;

FIG. 69A is a dorsal view of an alternative embodiment of the invention related to that shown in FIG. 10A;

FIG. 69B is a dorsal view of the device shown in FIG. 69A;

FIG. 70A is a dorsal view of an alternative embodiment of the invention related to that shown in FIG. 69A;

FIG. 70B is a dorsal view of the device shown in FIG. 70A;

FIG. 71 is a lateral view of the spine and an alternative embodiment of the invention related to that shown in FIG. 45;

FIG. 72A is lateral view of a knife-like instrument;

FIG. 72B is a lateral view of the spine and the cutting tool shown in FIG. 72A;

FIG. 73A is a lateral view of a tool used to distract the spinous processes;

FIG. 73B is a view of the one end of the distracting tool shown in FIG. 73A;

FIG. 73C is a lateral view of the tool shown in FIG. 72A;

FIG. 73D is a view of the dorsal aspect of two adjacent spinous processes and the end of the tool shown in FIG. 73C;

FIG. 73E is a dorsal view of two adjacent spinous processes and the tips of the tool shown in FIG. 73D;

FIG. 74A is a lateral view of a measuring tool;

FIG. 74B is a view of a gauge that may be used on the handle of the instrument shown in FIG. 74A;

FIG. 75 is an oblique view of a sleeve;

FIG. 76A is a lateral view of the spine and the embodiment of the invention shown in FIG. 12A;

FIG. 76B is a dorsal view of the spine and the embodiment of the invention shown in FIG. 76A;

FIG. 77A is a lateral view of the tip of an instrument;

FIG. 77B is a lateral view of the tip of the instrument shown in FIG. 77A;

FIG. 77C is a lateral view of the tip of the tool shown in FIG. 77A and a device according to the invention;

FIG. 78A is a lateral view of the tip of a distractor tool;

FIG. 78B is a dorsal view of the tips of two spinous processes and the tip of the distractor tool shown in FIG. 78A;

FIG. 79A is a dorsal view of the tip of a spinous process, a cross-section of a tool, and a cable;

FIG. 79B is a dorsal view of the tip of a spinous process, the cross-section of the tool shown in FIG. 79A and a cable;

FIG. 80A is a lateral view of the spine and an alternative embodiment of the invention related to that shown in FIG. 12A;

FIG. 80B is a dorsal view of the spine and the embodiment of the invention shown in FIG. 80A;

FIG. 80C is a cranial view of the embodiment of the invention shown in FIG. 80A;

FIG. 81 is a lateral view of the spine and an alternative embodiment of the invention related to that shown in FIG. 80A;

FIG. 82 is a caudal view including an alternative embodiment of the invention related to that shown in FIG. 81;

FIG. 83 is a caudal view including an alternative embodiment of the invention related to that shown in FIG. 82;

FIG. 84 is a lateral view of the spine and an alternative embodiment of the invention related to that shown in FIG. 37;

FIG. 85 is a dorsal view of the spine and an alternative embodiment of the invention shown in FIG. 80A;

FIG. 86 is a dorsal view of the spine and an alternative embodiment of the invention related to that shown in FIG. 85;

FIG. 87A is a dorsal view of the spine and an alternative embodiment of the invention related to that shown in FIG. 85;

FIG. 87B is a dorsal view of the spine and the embodiment of the invention shown in FIG. 87A;

FIG. 88A is a lateral view of the spine an alternative embodiment of the invention related to that shown in FIG. 20A;

FIG. 88B is a dorsal view of the spine and the embodiment of the invention shown in FIG. 88A;

FIG. 89A is a lateral view of the spine and an alternative embodiment of the invention related to that shown in FIG. 88A;

FIG. 89B is a dorsal view of the spine and the embodiment of the invention shown in FIG. 89A

FIG. 90A is an exploded oblique view of an alternative embodiment of the invention related to that shown in FIG. 10A;

FIG. 90B is a lateral view of the assembled device shown in FIG. 90A;

FIG. 90C is an anterior view of the assembled device shown in FIG. 90B;

FIG. 90D is coronal cross section of the assembled device shown in FIG. 90C;

FIG. 91A is a dorsal view of the spine and an alternative embodiment of the invention related to that shown in FIG. 12A;

FIG. 91B is a coronal cross-section of the spine and the embodiment of the device shown in FIG. 91A;

FIG. 92A is an oblique view of a shim-like device;

FIG. 92B is an exploded lateral view of the spine, shims, and an alternative embodiment of the invention related to that shown in FIG. 89A;

FIG. 92C is a dorsal view of the spine and the embodiment of the invention shown in FIG. 92B;

FIG. 93A is a lateral view of the spine and an alternative embodiment of the invention related to that shown in FIG. 61;

FIG. 93B is a dorsal view of the spine and the embodiment of the invention shown in FIG. 93A;

FIG. 93C is a dorsal view of the spine and an alternative embodiment of the invention related to that shown in FIG. 93B;

FIG. 94A is a view of the cranial side of the embodiment of the invention shown FIG. 10A and a novel insertion tool;

FIG. 94B is a side view of the embodiment of the invention shown in FIG. 94A;

FIG. 94C is a lateral view of the spine and the embodiment of the invention shown in FIG. 94B;

FIG. 94D is a lateral view of the spine and the embodiment of the invention shown in FIG. 94C;

FIG. 94E is an exploded lateral view of the spine and the embodiment of the invention shown in FIG. 94D;

FIG. 94F is an exploded view of the caudal end of a vertebra, and the embodiment of the tool shown in FIG. 94E;

FIG. 94G is a dorsal view of the spine and the embodiment of the invention shown in FIG. 94F;

FIG. 94H is a cross section of the embodiment of the invention shown in FIG. 94A;

FIG. 95A is a lateral view of the spine, the embodiment of the SPS shown in FIG. 10A, and a second impactor tool;

FIG. 95B is a dorsal view of the spine and the embodiment of the invention shown in FIG. 95A;

FIG. 95C is a lateral view of the tool shown in FIG. 95A;

FIG. 95D is a view of the cranial side of the tool shown in FIG. 95C;

FIG. 96A is a cranial view of an alternative embodiment of the invention related to that shown in FIG. 94A;

FIG. 96B is a lateral view of the embodiment of the invention shown in FIG. 96B;

FIG. 97A is a lateral view of an alternative embodiment of the invention related to that shown in FIG. 73A;

FIG. 97B is an exploded cranial view of the embodiment of the invention shown in FIG. 97A;

FIG. 97C is an oblique view of the embodiment of the invention shown in FIG. 97A and one arm of a McCulloch retractor;

FIG. 98A is an exploded oblique view of an alternative embodiment of the invention related to that shown in FIG. 20A;

FIG. 98B is an oblique view of an assembled device of the embodiment shown in FIG. 98A; and

FIG. 98C is a lateral view of the spine and the embodiment of the invention shown in FIG. 98A.

FIG. 99A is a dorsal view of the spine and an alternative embodiment of the invention including rods that connect components placed between spinous processes;

FIG. 99B is an exploded dorsal view of the embodiment of the invention drawn in FIG. 99B;

FIG. 99C is a lateral view of the spine and the embodiment of the invention drawn in FIG. 99B;

FIG. 100A is an oblique view of an alternative embodiment of the invention related to that drawn in FIG. 97A;

FIG. 100B is a dorsal view of the embodiment of the invention drawn in FIG. 100A;

FIG. 100C is a lateral view of the embodiment of the device drawn in FIG. 100B;

FIG. 101A is a coronal cross section of an alternative embodiment of the invention drawn in FIG. 93B;

FIG. 101B is sagittal cross section of the embodiment of the device drawn in FIG. 101A; and

FIG. 101C is a lateral view of the spine and the embodiment of the device drawn in FIG. 101A.

### SUMMARY OF THE INVENTION

This invention is directed to surgical apparatus for treating spinal stenosis, without the need for laminectomy. Broadly the invention resides in a device configured for placement posteriorly to a spinal canal between vertebral bodies to distract the spine and enlarge the spinal canal. In the preferred embodiments the device permits spinal flexion while limiting spinal extension, thereby providing an effective treatment for treating spinal stenosis. The invention may be used in the cervical, thoracic, or lumbar spine.

Numerous embodiments are disclosed, including elongated, length-adjustable components coupled to adjacent vertebral bodies using pedicle screws. The preferred embodiments, however, teach a device configured for placement between adjacent vertebrae and adapted to fuse to the lamina, facet, spinous process or other posterior elements of a single vertebra. Various mechanisms, including shape, porosity, tethers, and bone-growth promoting substances may be used to enhance fusion. The tether may be a wire, cable, suture, allograft tissue, or other single or multi-filament member. Preferably, the device forms a pseudo-joint in conjunction with the non-fused vertebra. Alternatively, the device could be fused to the caudal vertebra or both the cranial and caudal vertebrae.

In certain embodiments at least a portion of the device is constructed from bone.

For example, the device may be constructed from the shaft of the clavicle, rib, humerus, radius, ulna, metacarpal, phalanx, femur, tibia, fibula, or metatarsal bone. The device includes a slot or indent to receive a portion of a spinous process or other vertebral feature to enhance fusion. The device may contain one or more bone-growth promoting substances such as BMP1, 2, 3, 4, 5, 6, 7, 8, 9, 10, 11, 12, 13, 14 . . . n, demineralized bone matrix, allograft cancellous bone, autograft bone, hydroxy appetite, coral or other highly porous substance.

An elastic, synthetic ligament or allograft ligament may me provided as part of the invention. The device may be configured to surround or clamp to a single spinous process, or include optional projections extending along the sides of a spinous process.

The device may include spring-like or shape-memory properties. The device may have an asymmetric cross section or other shape to wedge or distract the spinous processes upon insertion. The may include a generally V-, U-, or C-shaped device configured to fit between the lamina of one vertebra and the spinous process and or lamina of an adjacent vertebra, and my be customized at the time of surgery.

The devices according to this invention may be made of any suitable material, including titanium, chrome-cobalt, stainless steel, polymers, liquid metals, shape-memory materials, ceramics, or human tissue. The device may be made of an in-situ curing material. The device could be customized to fit between the spinous processes. Bone or bone-growth material could be added top the device after the device cures.

Devices according to the invention may be constructed of bone, including allograft bone, PEEK (polyaryletherketone), or ceramic. Devices according to the invention may also me made of other biocompatible materials such as Polyphenolsulfone, Polysulfone, Acetal (Delrin), UHMW Polyethylene, and composites of these materials and carbon fibers. Alternative materials include bioresorbable materials such as polylactic acid (PLA), polyglycolic acid (PGA), poly (ortho esters), poly(glycolide-co-trimethylene carbonate), poly-L-lactide-co-6-caprolactone, polyanhydrides, poly-n-dioxanone, and poly(PHB-hydroxyvaleric acid).

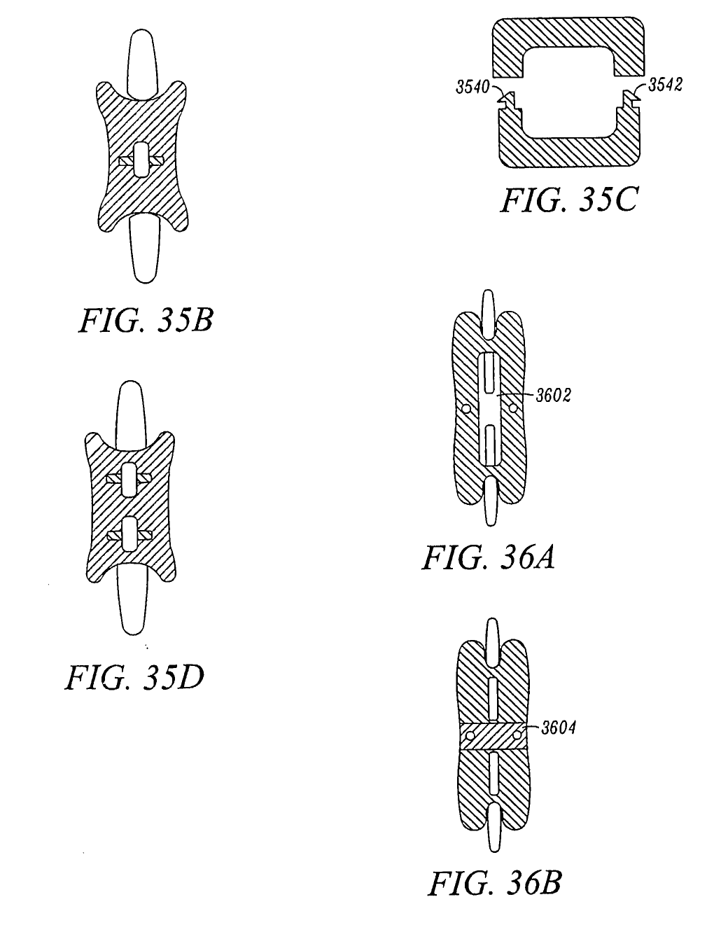

Certain devices according to the invention are designed to withstand loads of at least 90N, and are preferably provided in a number of sizes. For example, the cranial-to-caudal dimensions could vary from 6 mm-24 mm in 2 mm increments. The ventral-to-dorsal dimensions could also vary from 6 mm-24 mm, also in 2 mm increments. The left-to-right dimensions could vary from 10-50 mm, again in 2 mm increments. Multi-level devices, similar to the embodiment shown in FIG. 35D would be supplied in larger dimensions.

### DETAILED DESCRIPTION OF THE INVENTION

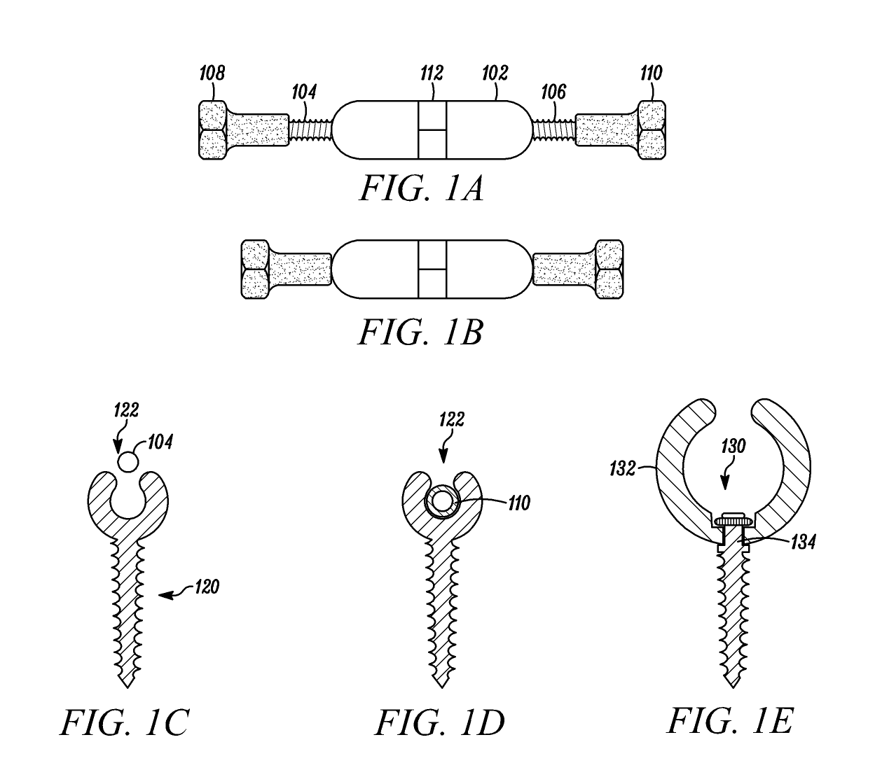

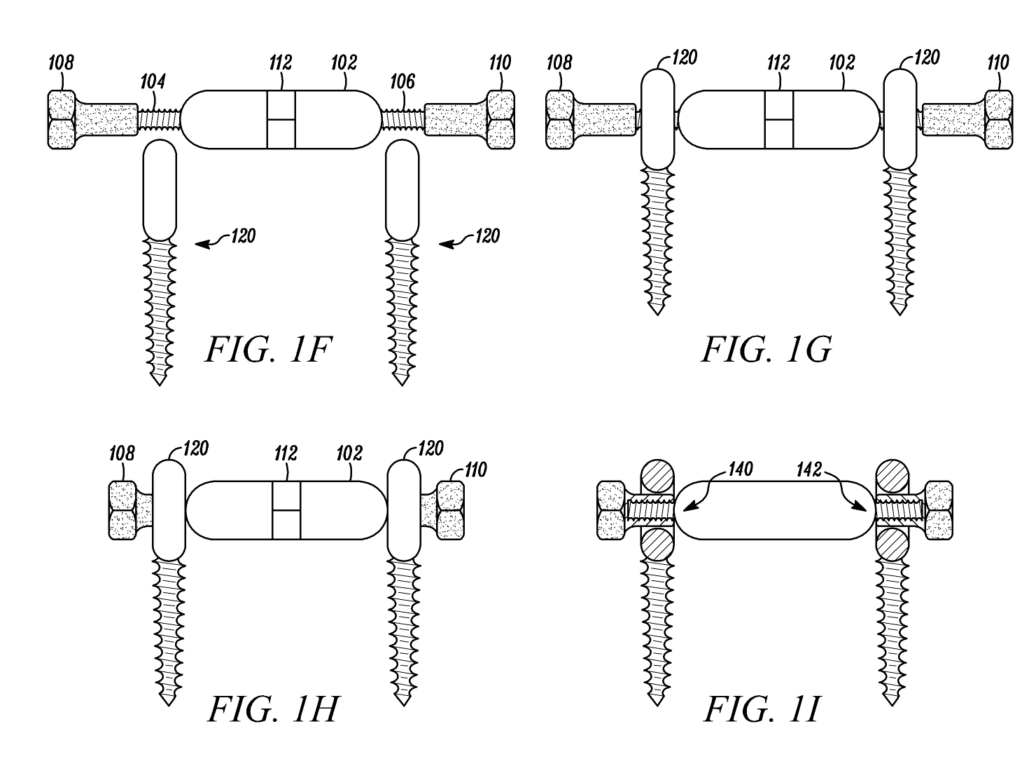

FIG. 1A is a lateral view of a three-component device used to treat spinal stenosis, drawn in its extended position. The central rod component 102 is threaded 104, 106 on both ends. One end of the component has left-handed threads. The other end of the rod component has right-handed threads. Bolt-like components 108, 110 are threaded onto the ends of the rod component. As discussed in further detail below, the rod component is coupled to pedicle screws then adjusted to force the screws apart. This permits spinal flexion, but limits spinal extension, thereby distracting the spine and enlarging the spinal canal.

FIG. 1B is a lateral view of the device of FIG. 1A drawn in its contracted position. Tools are used to prevent rotation of the end components. A wrench may be used to rotate the rod component placed on flats 112. Rotating the rod component, while preventing rotation of the end components, causes the end components to advance along the treaded portions of the rod, simultaneously.

FIG. 1C is an end view of a vertebral screw 120 and a cross section of the threaded portion 104 of the rod, which passes through an opening 122 in the screw. FIG. 1D is an end view of the screw drawn in FIG. 1C and a cross section of the threaded end component 110 drawn in FIG. 1A. The larger diameter of the end component prevents the component from passing through the opening in the screw.

FIG. 1E is an end view of an alternative embodiment of a screw having a mechanism 130 that permits a connector ring 132 to swivel about a shaft 134 of the screw.

FIG. 1F is a lateral view of the device drawn in FIG. 1A and the screws of the embodiment drawn in FIG. 1C. The device is drawn in its extended position. The narrow diameters of the treaded portions of the rod component permit the device to be inserted through the openings in the screws. FIG. 1G is a lateral view of the embodiment of the device drawn in FIG. 1F. The rod-like device has been inserted into the screws. FIG. 1H is a lateral view of the device drawn in FIG. 1G. The device is drawn in its contracted position. Rotation of the rod-like component advances the end bolt-like components into the screws. The large diameter of the end components prevents the tightened rod component from passing through the slot-like openings in the screws.

FIG. 1I is a sagittal cross section of the device drawn in FIG. 1H. The hemispherical ends 140, 142 of the rod component 102 articulate with the donut-like opening of the screws. The drawing illustrates the spherical cross section of the connector portion of the screws. The cooperation between the rod component and the screws prevent the heads of the screws from approaching one another. The enlargements at the ends of the assembled device may also be used to limit spinal flexion. The devices according to this invention may be made of any suitable material, including titanium, chrome-cobalt, stainless steel, polymers, liquid metals, shape-memory materials, ceramics, or human tissue. The device may also include a spring component. For example, a coil spring could be placed around the rod component.

The following disclosure describes how the device may be used to enlarge the spinal canal. The screws are placed into the pedicles of adjacent vertebrae, or into vertebrae spaced apart by at least one intermediate vertebra. The rod component is installed, and the enlargements at the ends of the bolt-like end components prevent the assembled rod from dissociating from the screws. A rod component of the appropriate length is selected to force the screws apart. This distracts the spine and enlarges the spinal canal as the heads of the screws separate. The circular openings in the screws enable the screws to slide along the end components. This permits spinal flexion, but limits spiral extension, which enlarges the spinal canal. Spinal flexion occurs as the screws advance along the end components. Spinal extension decreases the diameter of the spinal canal and decreases the size of the neuroforamina.

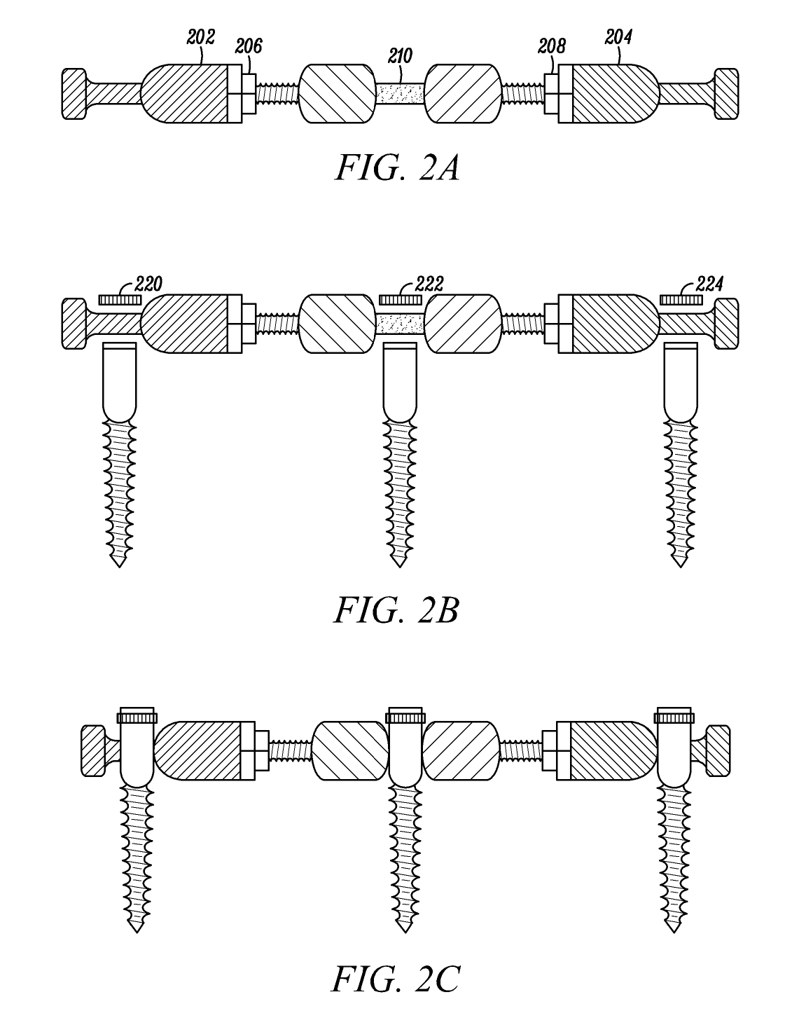

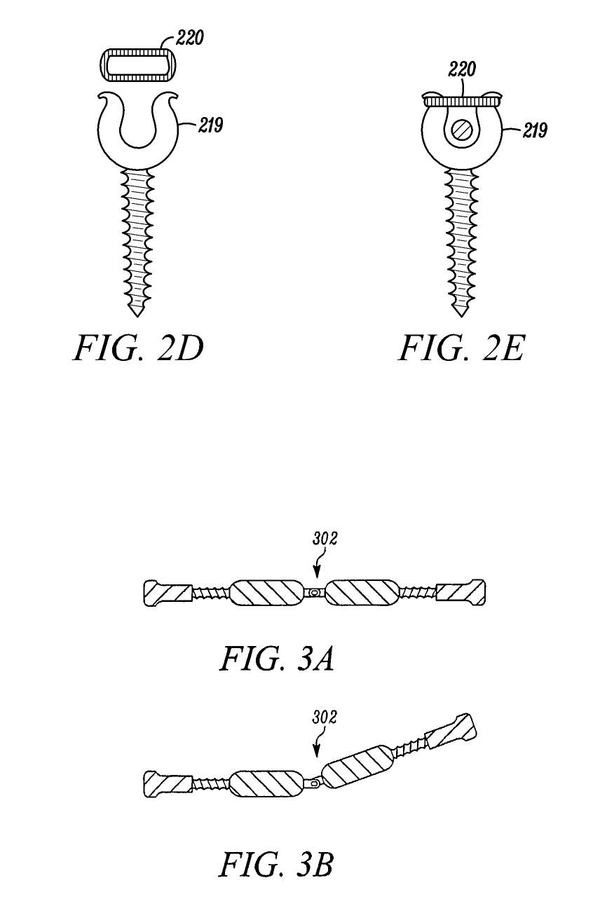

FIG. 2A is a lateral view of an alternative embodiment of the invention which has two rod-like components 202, 204. The rod components have a turnbuckle to allow lengthening and shortening of the rods. Each turnbuckle further includes a nut 206, 208 that permits the rod to be locked in a particular length. One end of the rod component is shaped somewhat like the end component drawn in FIG. 1F. The rods are connected to one another by a cable 210.

FIG. 2B is an exploded view of the device drawn in FIG. 2A and an alternative embodiment of the vertebral screws, each using a ring-like closure mechanism 220, 222, 224. FIG. 2C is a lateral view of the device drawn in FIG. 2B. The device has been drawn in an assembled configuration. FIG. 2D is an exploded end view of the screw 219 and a fastening component 220.

FIG. 2E is end view of the screw and fastening component drawn in FIG. 2D and a cross section of the rod-like device drawn in FIG. 2C. The fastening component may be made of a shape memory material. Alternatively, the fastening component may be made of an elastic material that is stretched prior to inserting the component. The fastening ring contracts after it is placed over the head of the screw.

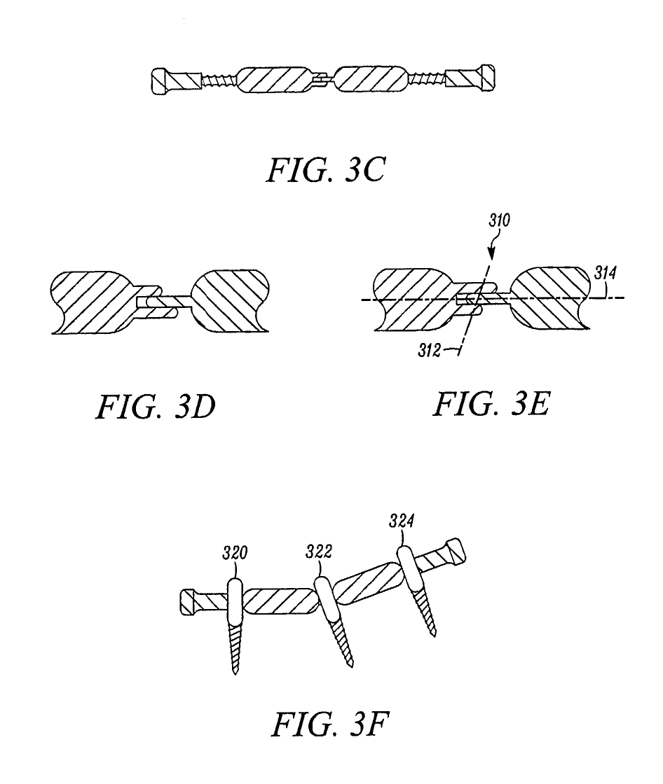

FIG. 3A is a lateral view of an alternative embodiment of the invention, wherein the rod-like components are connected by a hinge joint 302. FIG. 3B is a lateral view of the embodiment of the device drawn in FIG. 3A. The rods are drawn in a different position than drawn in FIG. 3A. FIG. 3C is a view of the top of the device drawn in FIG. 3B. FIG. 3D is an enlarged view of the top of the hinge joint drawn in FIG. 3C.

FIG. 3E is an enlarged view of the top of an alternative hinge joint 310 oriented in a direction 312 that is not perpendicular to the axis 314 of the rod components. FIG. 3F is a lateral view of the device drawn in FIG. 3B and vertebral screws 320, 322, 324.

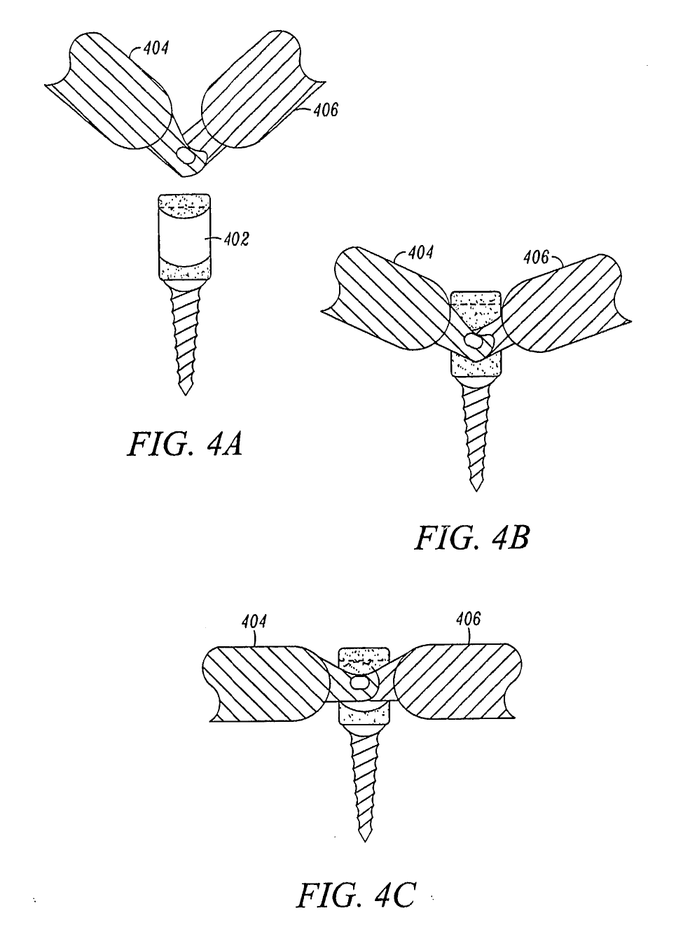

FIG. 4A is an exploded lateral view of an alternative vertebral screw and a hinged device that passes through an angled slot 402 in the screw when the rods 404, 406 are angled properly. FIG. 4B is a lateral view of the device with the rods area angled to pass the rods through the slot in the screw. FIG. 4C is a lateral view of the device with the rods oriented such that they will not pass through the slot in the screw.

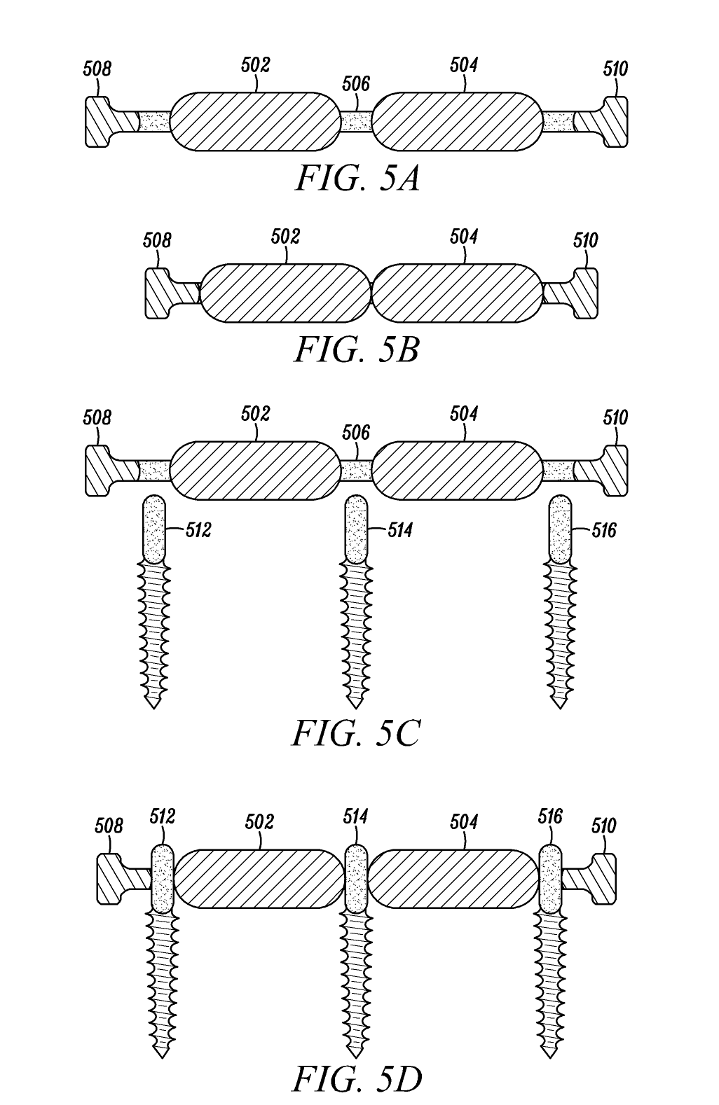

FIG. 5A is a lateral view of an alternative embodiment wherein rod-like components 502, 504 are threaded over an elastic cord 506. The end components 508, 510 are connected to the elastic cord. The device is drawn in its extended position. The elastic cord is stretched in the extended position. FIG. 5B is a lateral view of the device drawn in its contracted position. FIG. 5C is an exploded view of the device drawn in FIG. 5A and vertebral screws 512, 514, 516. The elastic cord passes through the slots in the vertebral screws. FIG. 5D is a lateral view of the assembled device drawn in FIG. 5C.

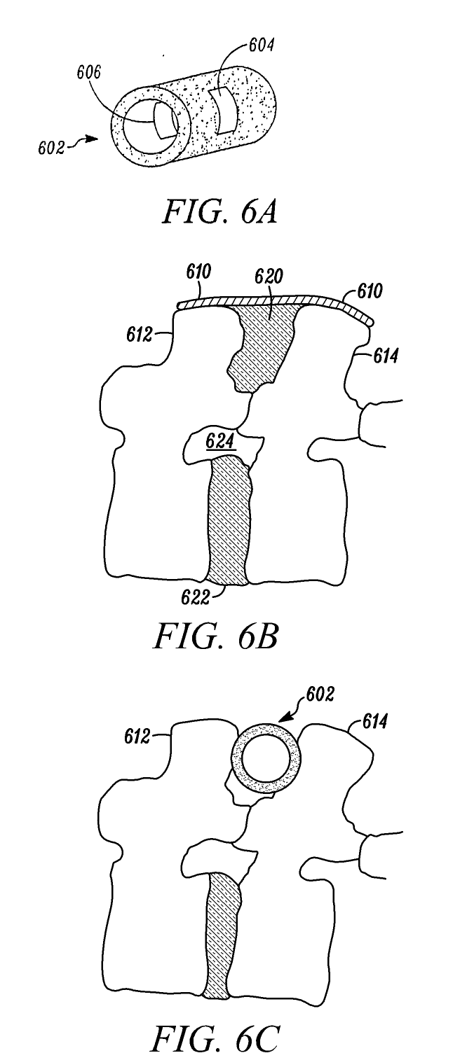

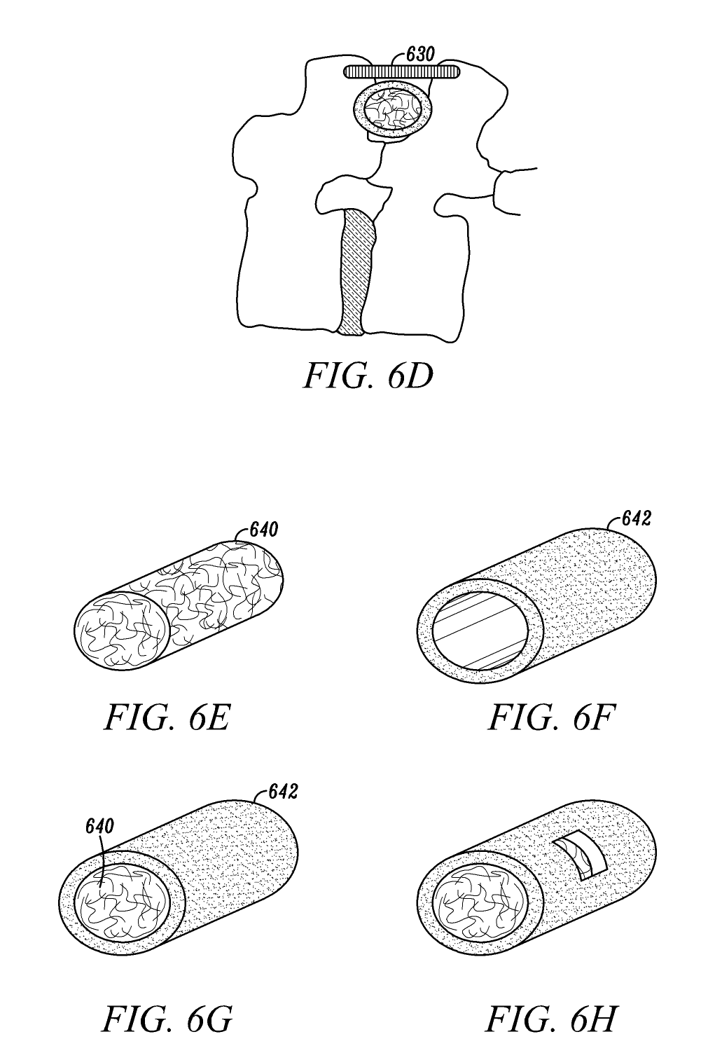

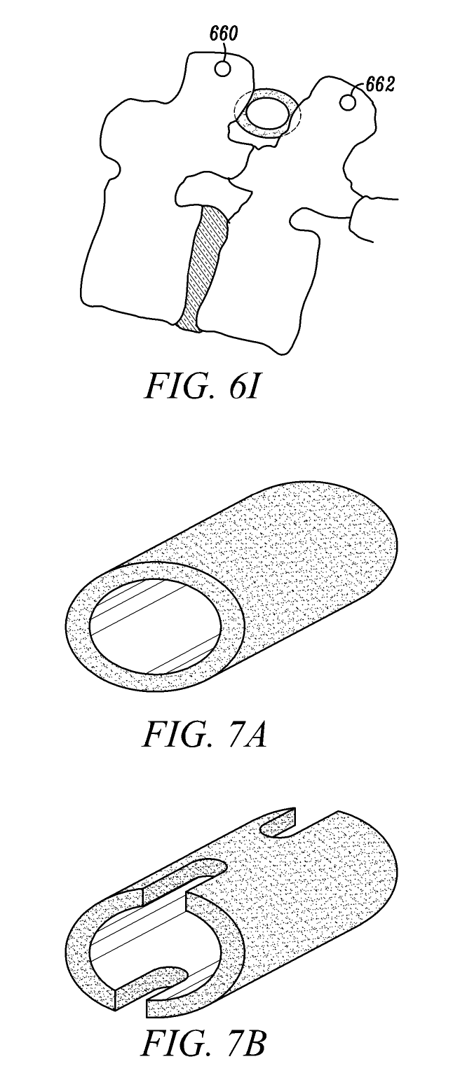

FIG. 6A is an oblique view of an alternative embodiment of the invention in the form of a cylindrical device 602 having two slots 604, 606 in sides of the device. The ends of the tube shaped device may by open. FIG. 6B is a lateral view of a portion of the spine. The supraspinous ligament 610 is attached to the dorsal surface of the spinous processes 612, 614 of two consecutive vertebrae. The interspinous ligament 620 courses between the spinous processes of the vertebrae. The intervertebral disc is depicted at 622 and the neuroforamina at 624.

FIG. 6C is a lateral view of a portion of the spine and the device of FIG. 6A, which has been wedged between the spinous processes. The supraspinous and interspinous ligaments have been removed. The device forces the spinous processes apart. The spine flexes as the spinous processes are forced apart. The neuroforamina and the spinal canal are enlarged as the spine is flexed. The device holds the vertebrae in a flexed position. The device may be made of any suitable materials, including bone, metals, ceramics, or polymers. For example, the device may be made from an allograft shaft of a long bone such as the humerus, tibia, fibula, radius, ulna, or femur. Alternatively, the device may be made of material known as PEEK.

FIG. 6D is a lateral view of a portion of the spine and the device drawn in FIG. 6A. The device has been filled with a material that promotes bone growth. For example, the device may be filled with bone, BMP soaked collagen sponges, or demineralized bone matrix. The device may fuse with one or both of the spinous processes. The device does not fuse with other portions of the vertebrae. For example, the device does not fuse across to the lamina of the vertebrae. The lamina of the vertebrae remain their normal size and shape. The lack of fusion across the lamina facilitates future surgical “decompression” procedures. The interspinous has been reconstructed. The area of the drawing at 630 represents the reconstructed interspinous ligament. Allograft tendon may be used to reconstruct the interspinous ligament. Other materials such as Gortex, Dacron, Marlex or other non-absorbable material may be used to reconstruct the interspinous ligament.

FIG. 6E is an oblique view of a cancellous bone block 640 which may be placed into the device drawn of FIG. 5A. FIG. 6F is an oblique view of a section of the shaft 642 of a long bone. FIG. 6G is an oblique view of a section of the shaft of a long bone and a cancellous bone block. The cancellous bone block has been placed into the cortical bone ring. FIG. 6H is an oblique view showing two slots machined into the sides of the cortical bone ring. As with the embodiment of FIG. 6A, the slots are shaped to fit over at least a portion of the spinous processes.

FIG. 6I is lateral view of a portion of the spine and a sagittal cross section of the embodiment of the device drawn in FIG. 6A or 6H. The dotted lines represent the outline of the cortical ring. The drawing illustrates holes 660, 662 in the spinous processes receive an allograft tendon. Allograft tissue could also be wrapped around the cranial aspect of the cranial spinous process and the caudal aspect of the caudal spinous process.

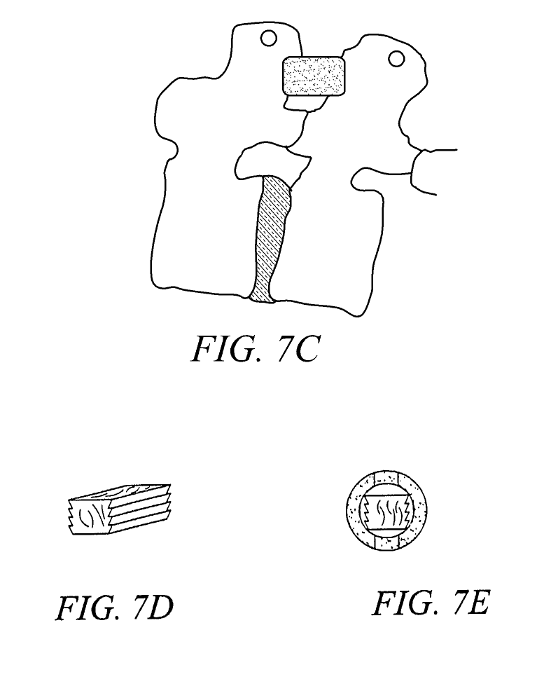

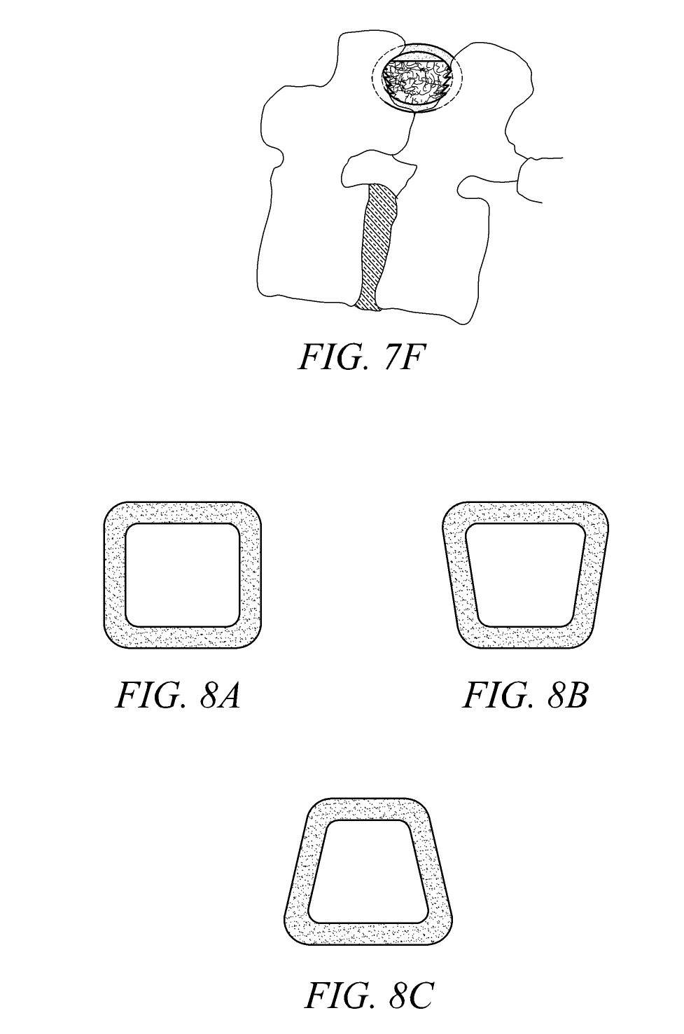

FIG. 7A is an oblique view of a portion of a shaft of a long bone. FIG. 7B is an oblique view of portion of a shaft of a long bone that has been machined to fit between two spinous processes. The bone has been machined to insert the device at an orientation ninety degrees to the orientation drawn in FIG. 6I. FIG. 7C is a lateral view of the spine and the device of FIG. 7B which has been inserted between the spinous processes.

FIG. 7D is an oblique view of a piece of bone with teeth machined or otherwise formed to facilitate insertion in a first direction and resist extrusion in a direction 180 degrees from the first direction. FIG. 7E is an end view of the device drawn in FIG. 7A and the bone drawn in FIG. 7D which has been inserted into the device drawn in FIG. 7B. The bone of FIG. 7D may be fastened to the device of FIG. 7B.

FIG. 7F is a lateral view of a portion of the spine and a sagittal cross section of the embodiment of the device drawn in FIG. 7E. The dotted lines represent the outline of the periphery of the device.

FIG. 8A is an end view of an alternative shape of the device drawn in FIG. 7B. FIG. 8B is an end view of an alternative shape of the device drawn in FIG. 8A. FIG. 8C is an end view of an alternative shape of the device drawn in FIG. 8B. Alternative shapes are possible, including solid forms.

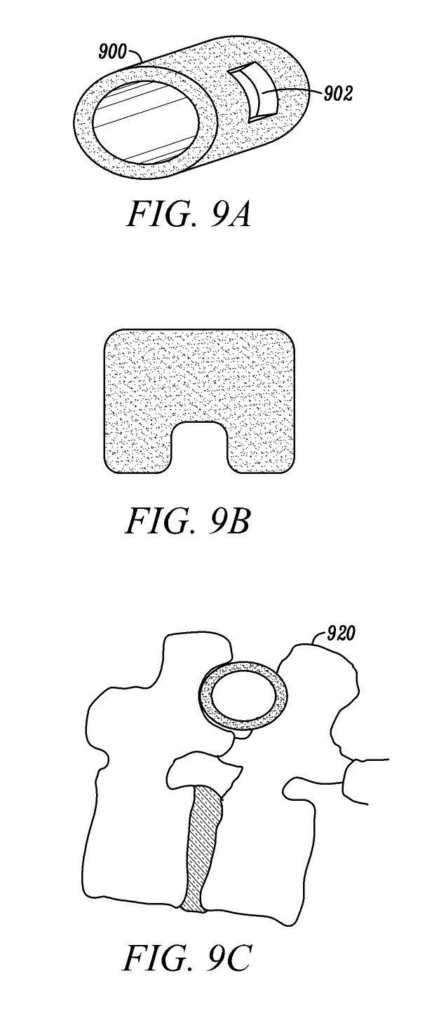

FIG. 9A is an oblique view of an alternative embodiment of the invention which has a single slot 902 on one side of the device 900. FIG. 9B is a view of the top of the embodiment of the invention drawn in FIG. 8A. FIG. 9C is a lateral view of a portion of the spine and the embodiment of the invention drawn in FIG. 9A. The device straddles a single spinous process 920. This embodiment of the device is designed to fuse to a single spinous process. Fusion to a single spinous process allows spinal flexion, but limits spinal extension. The invention anticipates embodiments of the device that do not fuse to either spinous process.

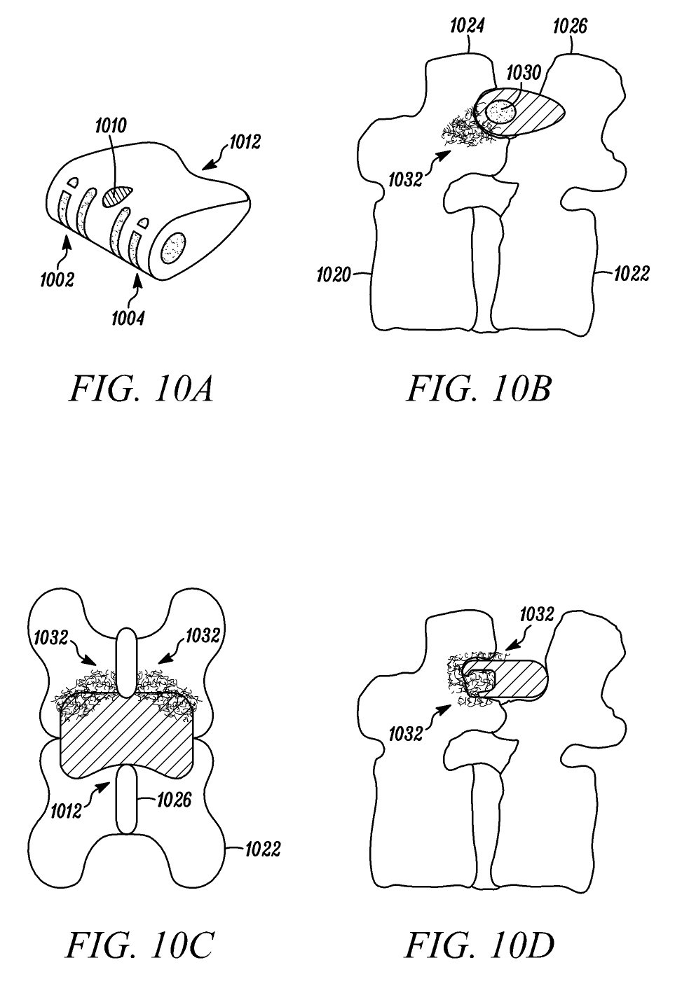

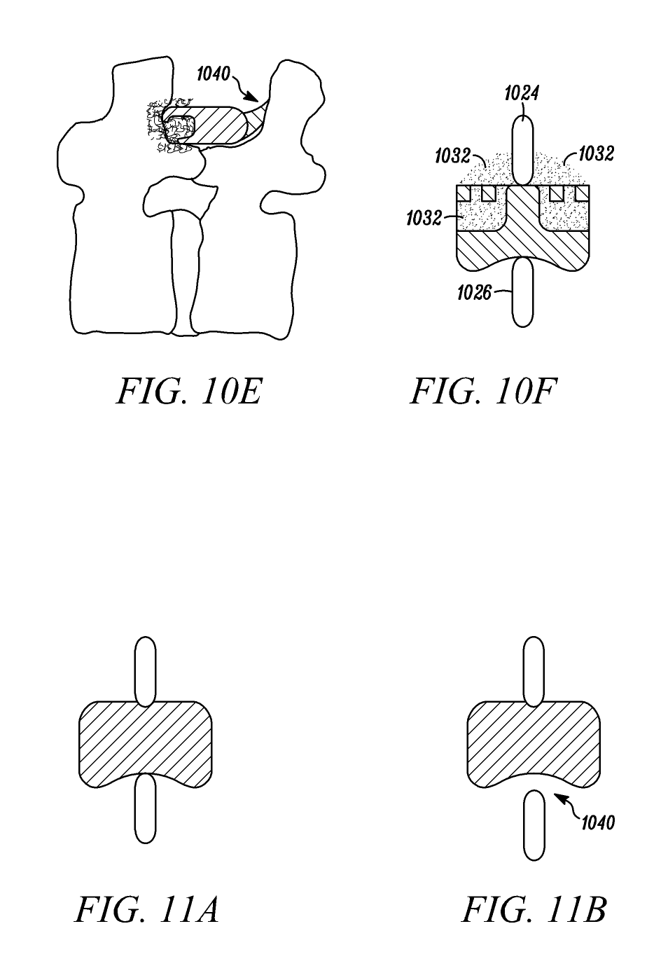

FIG. 10A is an oblique drawing of an alternative embodiment of the invention related to that shown in FIG. 6A. The device has chambers 1002, 1004 in the left and right sides of the cranial portion which may be filled with a material that promotes the growth of bone into the device. One or more openings may connect the two chambers. The cranial end of the device also has openings that extend into the chambers. The openings provide a path for cells to migrate into the chambers. The device has a notch 1010 on its cranial side which may or may not have teeth.

The notch accommodates the Spinous Process (SP) of the cranial vertebra. The caudal end 1012 of the device preferably includes a concavity. As with the embodiment of FIG. 6C, the device may be made of bone including allograft bone, metal such as titanium, PEEK (polyaryletherketone), or ceramic. Devices according to the invention may also me made of other biocompatible materials such as Polyphenolsulfone, Polysulfone, Acetal (Delrin), UHMW Polyethylene, and composites of these materials and carbon fibers. Alternative materials include bioresorbable materials such as polylactic acid (PLA), polyglycolic acid (PGA), poly (ortho esters), poly(glycolide-co-trimethylene carbonate), poly-L-lactide-co-6-caprolactone, polyanhydrides, poly-n-dioxanone, and poly(PHB-hydroxyvaleric acid).

Devices according to the invention are designed to withstand loads of at least 90N, and are preferably provided in a number of sizes. For example, the cranial-to-caudal dimensions could vary from 6 mm-24 mm in 2 mm increments. The ventral-to-dorsal dimensions could also vary from 6 mm-24 mm, also in 2 mm increments. The left-to-right dimensions could vary from 10-50 mm, again in 2 mm increments. Multi-level devices, similar to the embodiment shown in FIG. 35D would be supplied in larger dimensions.

FIG. 10B is a lateral view of a portion of the spine and the embodiment of the invention shown in FIG. 10A. The device fits between the SP of two adjacent vertebrae 1020, 1022. The device distracts the spinous processes 1024, 1026. The device also causes relative flexion of the spine at the area of the spine treated with the device. The device has been filled with a bone growth promoting substance 1030. The bone growth material has also been applied to the lamina 1032 of the cranial vertebra 1020. A portion of the lamina of the cranial vertebra has been decorticated to facilitate migration of cells from the patient's bone to the bone growth material. The device is designed to fuse to the cranial vertebra. The lack of bone growth material at the caudal end of the device inhibits fusion to the caudal vertebra.

FIG. 10C is a dorsal view of a portion of the spine and the embodiment of the invention shown in FIG. 10A. The SP 1026 of the caudal vertebra 1022 fits into the concavity 1012 on the caudal end of the device. The SP 1024 of the cranial vertebra 1020 fits into the notch 1010 on the cranial end of the device. Bone growth material 1032 is shown in the cranial aspect of the device and the lamina, SP, and facets of the cranial vertebra.

FIG. 10D is a sagittal cross section of the embodiment of the invention shown in FIG. 10B and a lateral view of the spine. The bone growth material 1032 can be seen extending from the lamina and SP of the cranial vertebra into one of the chambers in the device. The bone growth material extends through the slots in the cranial aspect of the device and through the openings on the left and the right sides of the device.

FIG. 10E is a sagittal cross section of the embodiment of the invention shown in FIG. 10D and a lateral view of the spine. The spine has been flexed. A gap 1040 can be seen between the SP of the caudal vertebra and the caudal end of the device.

FIG. 10F is a coronal cross section of the embodiment of the invention shown in FIG. 10E and the spine. The device is seated between the spinous processes of the cranial and caudal vertebrae. The chambers that house the bone growth material can be seen on the left and right sides of the device. Bone growth material 1032 can be seen passing through the slots on the cranial aspect of the device. An opening could connect the chambers in the left and the right sides of the device.

FIG. 11A is a dorsal view of the embodiment of the invention shown in FIG. 10F and the spine. FIG. 11B is a dorsal view of the embodiment of the invention shown in FIG. 11A and the spine. The spine has been flexed beyond the flexion caused by the device. A gap 1040 forms between the device and the caudal SP. The device is fused to the cranial vertebra. Alternatively, the device could be fused to the caudal vertebra or both the cranial and caudal vertebrae.

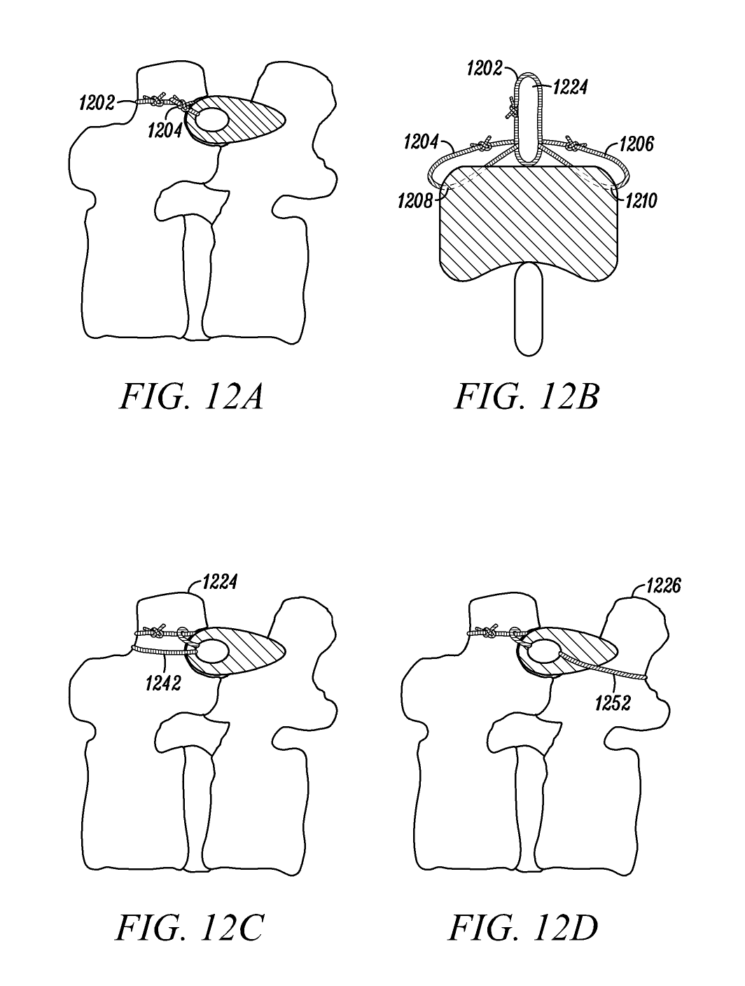

FIG. 12A is a lateral view of the spine and the embodiment of the invention shown in FIG. 10A. The device has been connected to the SP of the cranial vertebra. A cable, strap, cable tie, wire, cord, suture or other member 1202 has been wrapped around the base or waist of the SP. Second and third strap members 1204, 1206 pass between the SP and the loop around the SP. The second and third strap members are looped through holes 1208, 1210 on the left and right sides of the cranial aspect of the device. The device is forced into the SP, lamina, and/or facet joints of the cranial vertebra. The strapping method prevents migration of the device. The strapping method also prevents or restricts movement between the device and the cranial vertebrae. Reducing movement between the device and the cranial vertebra facilitates fusion to the cranial vertebra. The caudal end of embodiments of the device that are made of allograft bone could be treated to discourage fusion between the device and the caudal vertebra. For example, bone wax could be applied to the caudal end of the allograft device. Alternatively, the caudal end of the device could be covered with an allograft soft tissue, such as fascia, to inhibit bone growth to the device. Synthetic materials could also be used to inhibit bone growth to a portion of the device.

FIG. 12B is a dorsal view of the embodiment of the invention shown in FIG. 12A and the spine. A cable 1202 has been wrapped around the SP 1224 of the cranial vertebra. The second and third cables 1204, 1206 can be seen passing through the left and right sides of the device. The second and third cables also pass between the SP and the first cable. The cable that is looped around the SP of the cranial vertebra is preferably passed between the interspinous ligament and the cranial aspect of the SP.

FIG. 12C is a lateral view of the spine and the embodiment of the invention wherein a fourth cable, strap, cable tie, wire, cord, suture or other member 1242 has been passed around the SP 1224 of the cranial vertebra and through the device. The fastening devices are preferably made of non-absorbable material. Alternatively, fastening member 1242 could pass through a set of holes in the ventral portion of the device.

FIG. 12D is a lateral view of the spine and an embodiment of the invention wherein a fourth member 1252 has been passed around the SP 1226 of the caudal vertebra and through the device. The fourth cable may be tightly tied or loosely tied to permit movement between the caudal vertebra and the device.

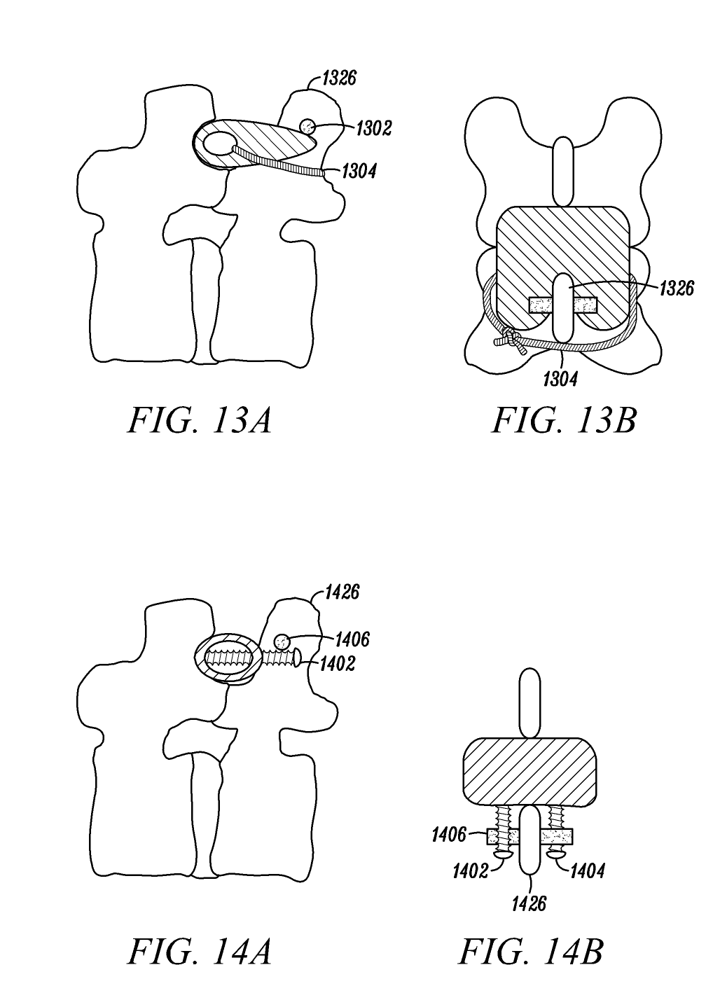

FIG. 13A is a lateral view of the spine and an embodiment of the invention wherein a screw, nail, or pin 1302 has been passed through the SP 1326 of the caudal vertebra. A member 1304 has been passed through the device and around the SP 1326 of the caudal vertebra. The cable and the pin prevent migration of the device. The cable and the pin also prevent or restrict movement between the device and the caudal vertebra. The device and the fastening method are designed to fuse the device to only the caudal vertebra. The caudal aspect of the device has holes that extend from the lamina and SP of the caudal vertebra to the chambers inside the device. Bone growth material is placed into the device and over the caudal vertebra. The lamina and/or the SP of the caudal vertebra could be decorticated to promote fusion. FIG. 13B is a dorsal view of the embodiment of the invention shown in FIG. 13A and the spine.

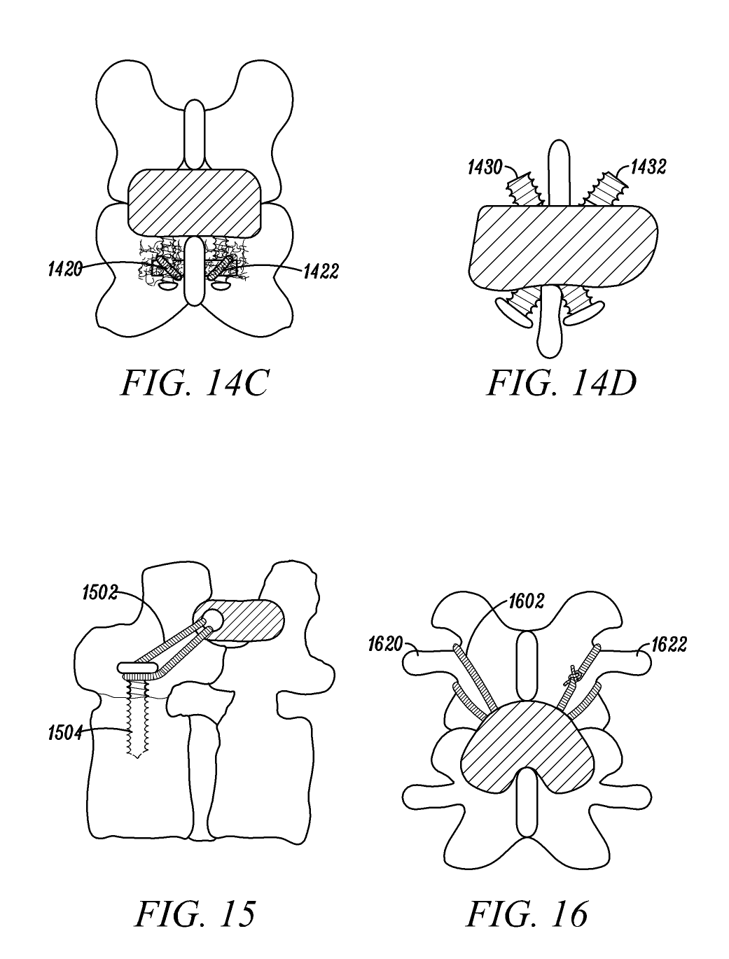

FIG. 14A is a lateral view of the spine and an alternative embodiment of the invention wherein screws 1402, 1404 pass through the caudal aspect of the device. A screw, pin, or nail 1406 also passes through the SP 1426 of the caudal vertebra. Note that transverse pin 1406 passes dorsal to one of the screws and ventral to the other screw.

FIG. 14B is a dorsal view of the embodiment of the invention shown in FIG. 14A and the spine.

FIG. 14C is a dorsal view of the spine and an embodiment of the invention wherein optional cables, sutures, wires, cable ties or like members 1420, 1422 have been wrapped around the screws and the pin. Bone growth material has been placed over the caudal aspect of the device and the SP and lamina of the caudal vertebra.

FIG. 14D is a dorsal view of the spine and an alternative embodiment of the invention wherein crossing screws 1420, 1432 pass through the device.

FIG. 15 is a lateral view of the spine showing an alternative method is used to fasten a device according to the invention to the cranial vertebra. A wire, cable, suture, or other single or multi-filament member 1502 is passed through the device and around or through screws 1504 placed into the pedicles of the cranial vertebra.

FIG. 16 is a dorsal view of the spine showing an alternative method used to fasten a device according to the invention to the cranial vertebra. A wire, cable, suture, or other single or multi-filament member 1602 is passed through the device and around the transverse processes 1620, 1622 of the cranial vertebra.

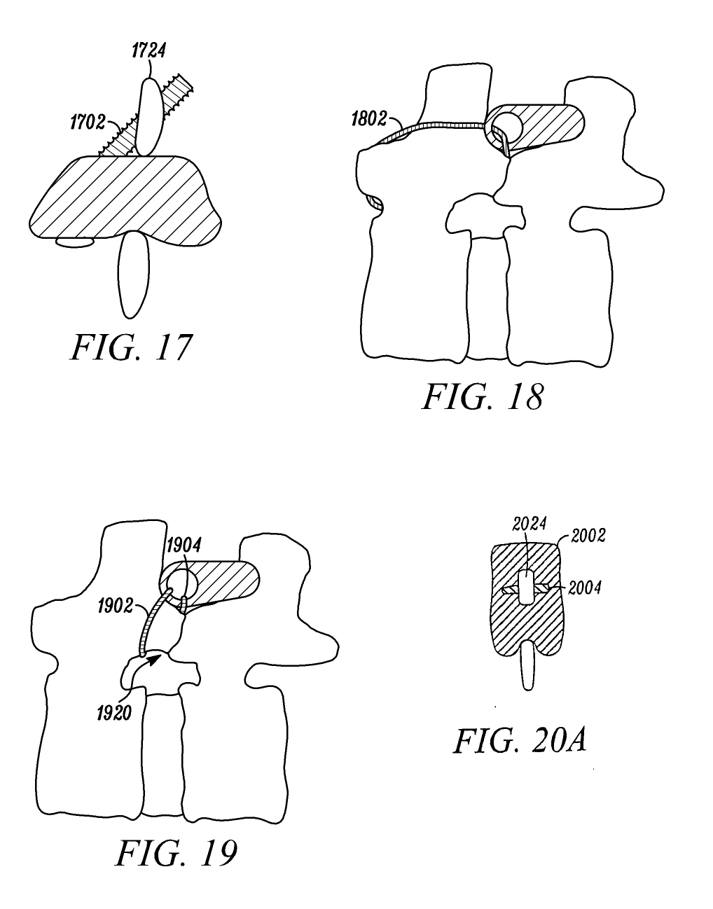

FIG. 17 is a dorsal view of the spine showing an alternative method used to fasten a device according to the invention to the cranial vertebra. A screw 1702 is passed through the device and through the cranial SP 1724. Alternatively, screws could be passed through the SP and the left and right sides of the device. The left and right sides of the device are preferably tapered.

FIG. 18 is a lateral view of the spine illustrating an alternative method used to fasten a device according to the invention to the cranial vertebra. Member 1802 passes through the left and right sides of the device. The member also passes around the lamina of the cranial vertebra.

FIG. 19 is a lateral view of the spine showing an alternative method used to fasten a device according to the invention to the cranial vertebra. Members 1902, 1904 pass through the left and right sides of the device. The members also pass around the cranial vertebra just cranial to the inferior facet joints 1920.

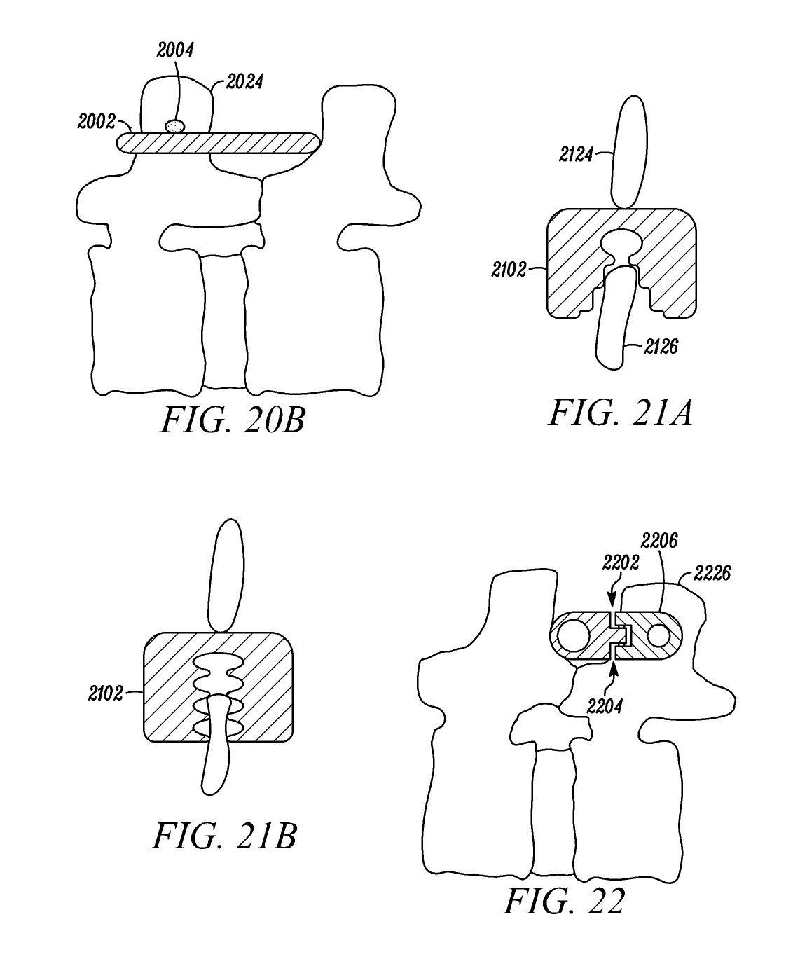

FIG. 20A is a dorsal view of the spine and an alternative embodiment of the invention including a device 2002 that surrounds the SP of the cranial vertebra. The device impinges against the cranial aspect of the caudal vertebra. The device may be held in place by a pin 2004 that passes through the SP 2024 of the cranial vertebra. FIG. 20B is a lateral view of the spine and the embodiment of the invention shown in FIG. 20A. The device is preferably designed to fuse to the cranial vertebra.

FIG. 21A is a dorsal view of the spine and an alternative embodiment of the invention 2102 which clamps to the cranial aspect of the SP of the caudal vertebra 2126. The device may also clamp to the cranial vertebra 2124, or both the caudal and cranial. The device may have spring properties that clamp the device to the SP. Alternatively, the device could be made of a shape memory material such as a Nitinol. The device could contract as it reacts to temperature change. FIG. 21B is a dorsal view of the spine and the embodiment of the invention shown in FIG. 21A. The device was shown in its clamped or contracted shape.

FIG. 22 is a lateral view of the spine and an alternative embodiment of the invention 2202 attached to the SP 2226 of the caudal vertebra. A hinge joint 2204 connects the fastener to the portion of the device that contains the bone growth material. A screw 2206 passes through the fastener component and the SP.

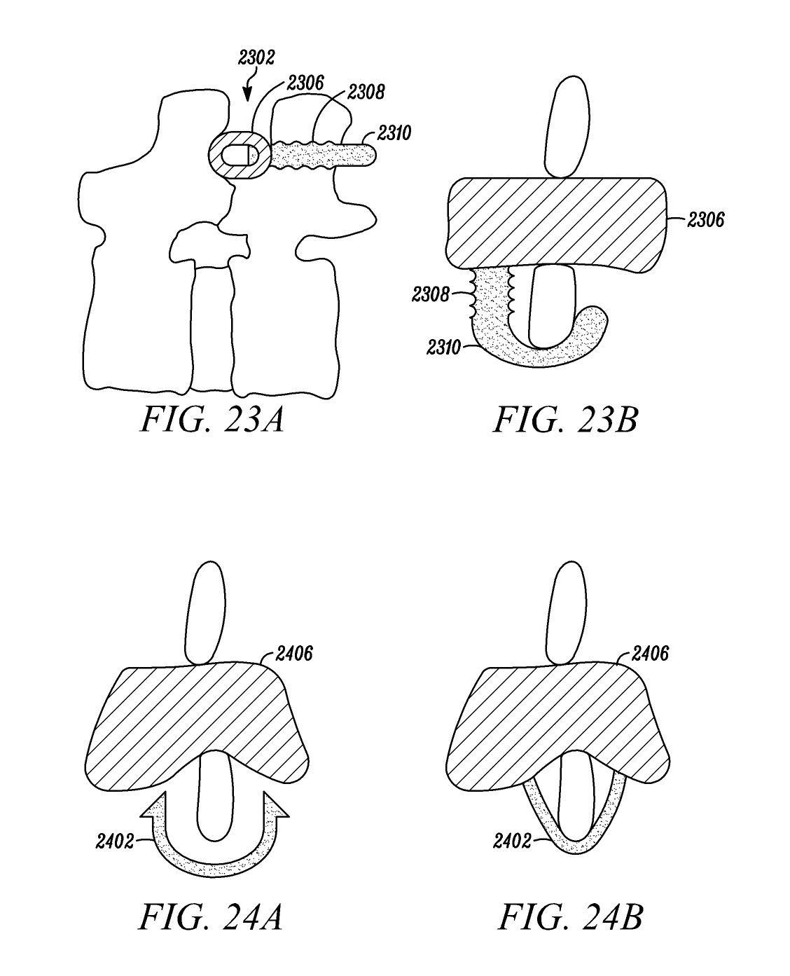

FIG. 23A is a lateral view of the spine and an alternative embodiment of the invention 2302 attached to the SP 2326 of the caudal vertebra. The device has a component 2306 that houses the bone growth material and a fastening component 2308.

FIG. 23B is a dorsal view of the spine and the embodiment of the invention shown in FIG. 23A. A hook 2310 from the fastening component is placed over the caudal aspect of the SP of the caudal vertebra. The fastening component may be connected to the component that contains the bone growth material via a ratchet mechanism. The ratchet mechanism locks the components after the components are compressed together.

FIG. 24A is an exploded dorsal view of the spine and an alternative embodiment of the invention including a spring-like clip 2402 that connects the device to the SP of the vertebra. FIG. 24B is a dorsal view of the spine and the embodiment of the invention shown in FIG. 24A. The spring-like component has been connected to the component 2406 that houses the bone growth material.

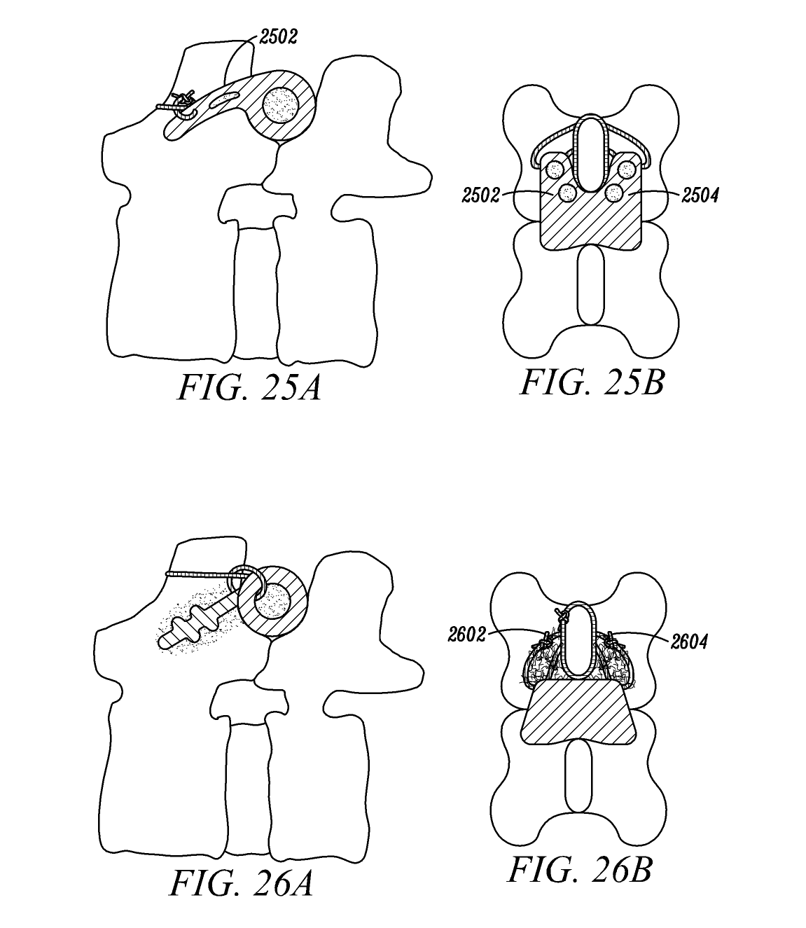

FIG. 25A is a lateral view of the spine and an alternative embodiment of the invention having projections 2502, 2504 that extend from the left and right sides of the cranial portion of the device. The projections have concavities that may receive bone growth promoting substances. The projections lie over the lamina of the cranial vertebra. The lamina may be decorticated to facilitate fusion between the device and the cranial vertebra. FIG. 25B is a dorsal view of the spine and the embodiment of the invention shown in FIG. 25A.

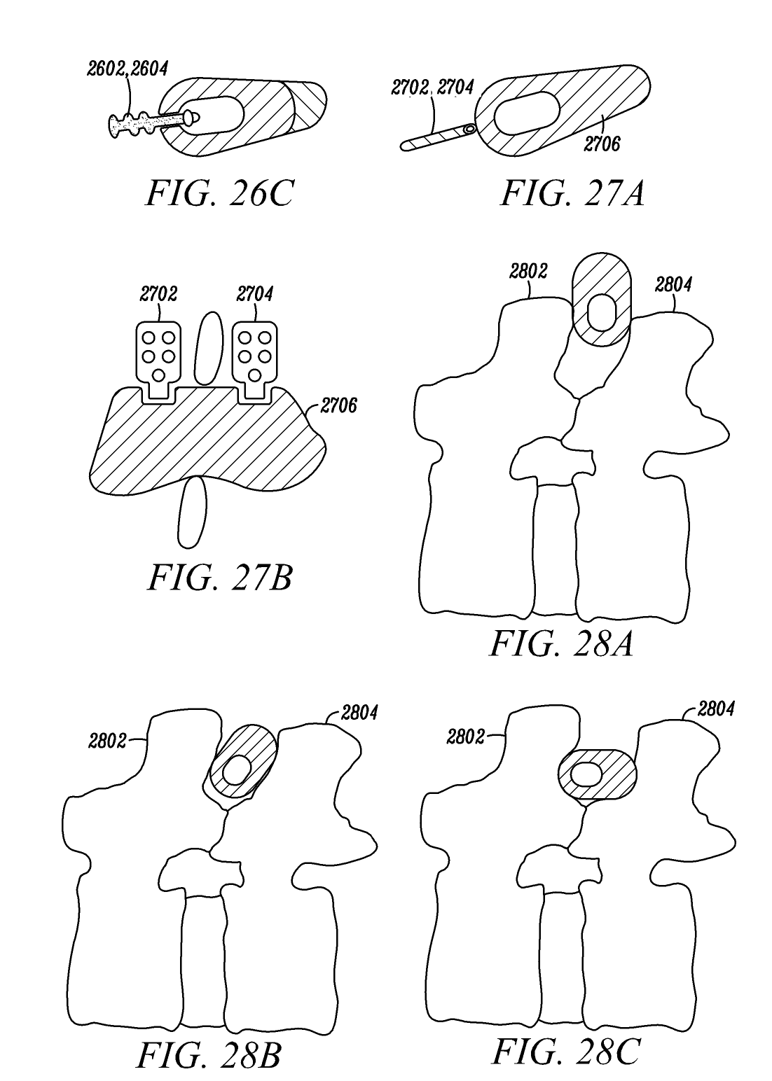

FIG. 26A is a lateral view of the spine and an alternative embodiment of the invention which has projections 2602, 2604 that extend from the left and right sides of the cranial portion of the device. Bone growth promoting substance has been packed around the projections. The projections have bristles that help hold the bone growth material. FIG. 26B is dorsal view of the spine and the embodiment of the invention shown in FIG. 26A. FIG. 26C is a sagittal cross section of an alternative embodiment of the invention shown in FIG. 26B. The projections 2602/4 swivel in holes on the cranial portion of the device.

FIG. 27A is a lateral view of an alternative embodiment of the invention, wherein projections 2702, 2704 from the cranial aspect of the device are connected to the component 2706 that houses the bone growth material via hinge joints. FIG. 27B is a dorsal view of the spine and the embodiment of the invention shown in FIG. 27A. The projections are preferably perforated to promote bone growth for a firmer attachment. Whereas a friction-fit or the use of soft tissues such as ligaments may weaken with time, fusion provides a more permanent attachment mechanism.

FIG. 28A is a lateral view of the spine illustrating one method of inserting a device according to the invention, including that shown in FIG. 10A. The spinous processes 2802, 2804 of the cranial and caudal vertebra are distracted as the wedge-shaped device is forced between the spinous processes. In FIG. 28B the spinous processes have been distracted by the device. In FIG. 28C the device has been rotated 90 degrees. The spinous processes have been further distracted as the device cams open the interspinous space.

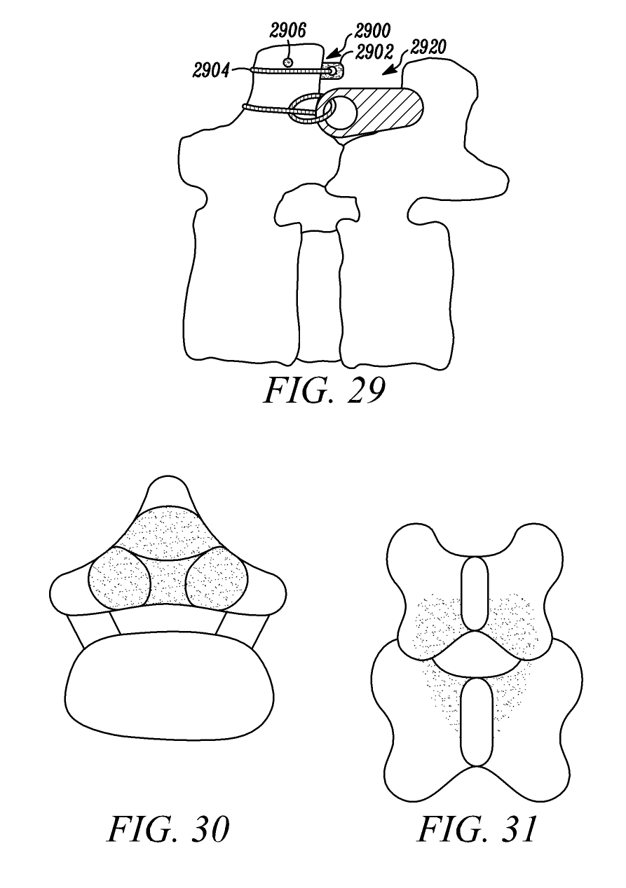

FIG. 29 is a lateral view of the spine along with an embodiment of the invention, such as that shown in FIG. 12A, including a device 2900 to help prevent extrusion of the spinous process spacer. The accessory device 2902 is strapped at 2904 to the SP of the cranial vertebra. A pin 2906 is placed through the SP dorsal to the strap 2904 of the accessory device 2902. The accessory device 2902 impinges against the dorsal aspect of the spacer device 2920 if the spacer device 2920 migrates in a dorsal direction.

FIG. 30 is a view of the caudal aspect of the cranial vertebra shown in FIG. 28C. The shaded area of the drawing represents possible contact points of the spinous process spacer shown in FIG. 12A. The spinous process spacer may contact the SP, lamina, and/or inferior facets of the cranial vertebra. FIG. 31 is a dorsal view of the spine. The shaded areas represent possible contact points of the spinous process spacer (SPS). The areas could be decorticated to promote fusion of the spinous process spacer to either or both vertebrae.

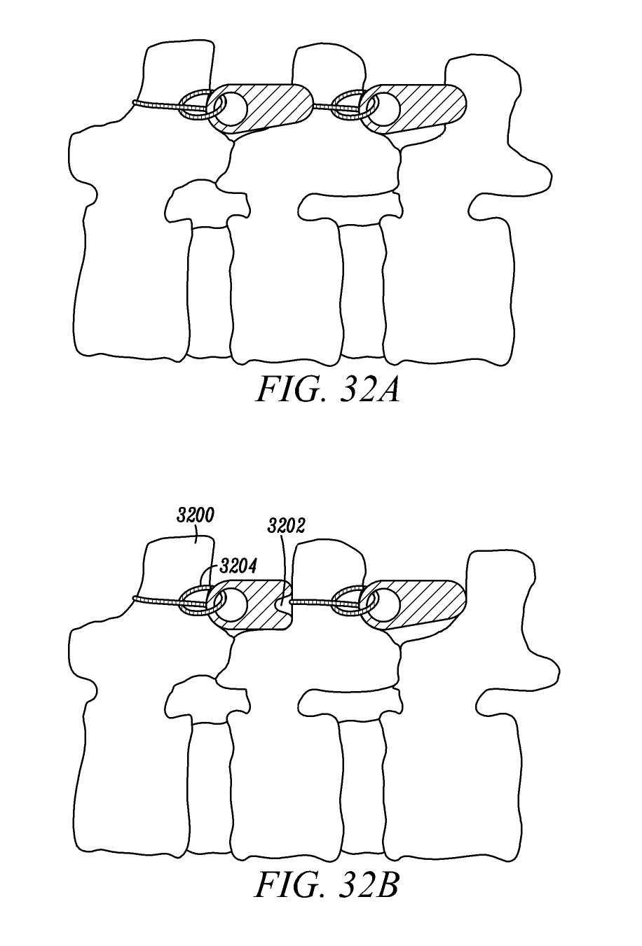

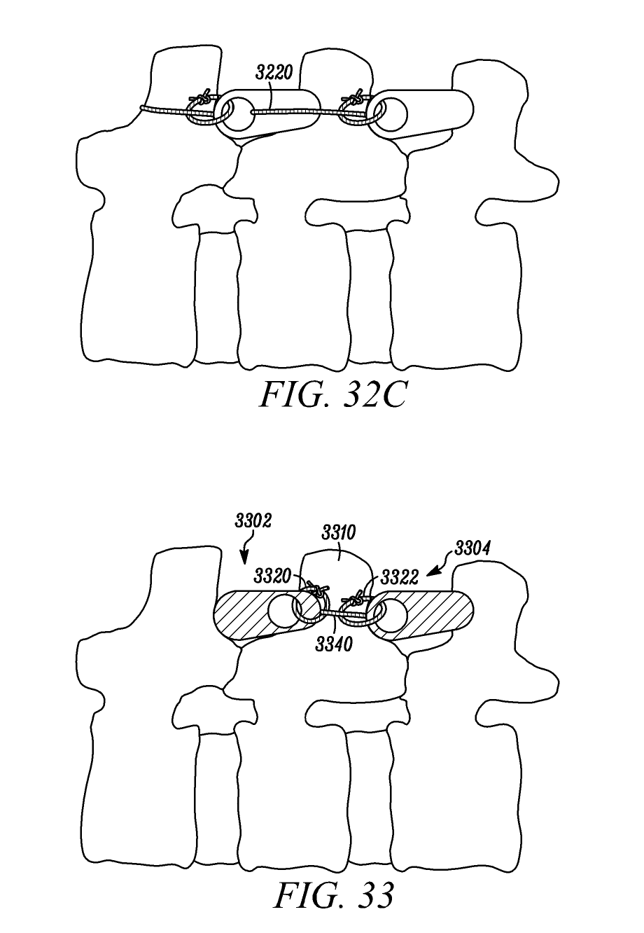

FIG. 32A is a lateral view of the spine and the embodiment of the invention shown in FIG. 12A. Spinous process spacers are used to distract two levels of the spine. Three or more spinous process spacers could be used to distract three or more levels of the spine. FIG. 32B is a lateral view of the spine and a variation of the embodiment of the invention shown in FIG. 32A. The caudal aspect of the cranial SPS has a concavity 3202. The strap 3204 from the caudal SPS 3200 fits in the concavity of the cranial SPS. The concavity avoids impingement of the strap from the caudal SPS between the cranial SPS and the intermediate SP.

FIG. 32C is a lateral view of the spine and an alternative, multilevel embodiment of the invention. The cranial strap 3220 from the caudal SPS is passed through an opening in the cranial SPS. The method avoids impingement of the strap from the caudal SPS and the intermediate SP.

FIG. 33 is a lateral view of the spine and an alternative, multilevel embodiment of the invention wherein SPS devices are connected to the cranial and caudal aspects of the SP of the intermediate vertebra. Both SPS devices 3302, 3304 are preferably fused to only the intermediate vertebra 3310. Cables are passed from the left and right sides of both SPS devices. The cables 3320, 3322 from the lateral aspects of the SPSs also pass through a cable 3340 wrapped around the SP of the intermediate vertebra.

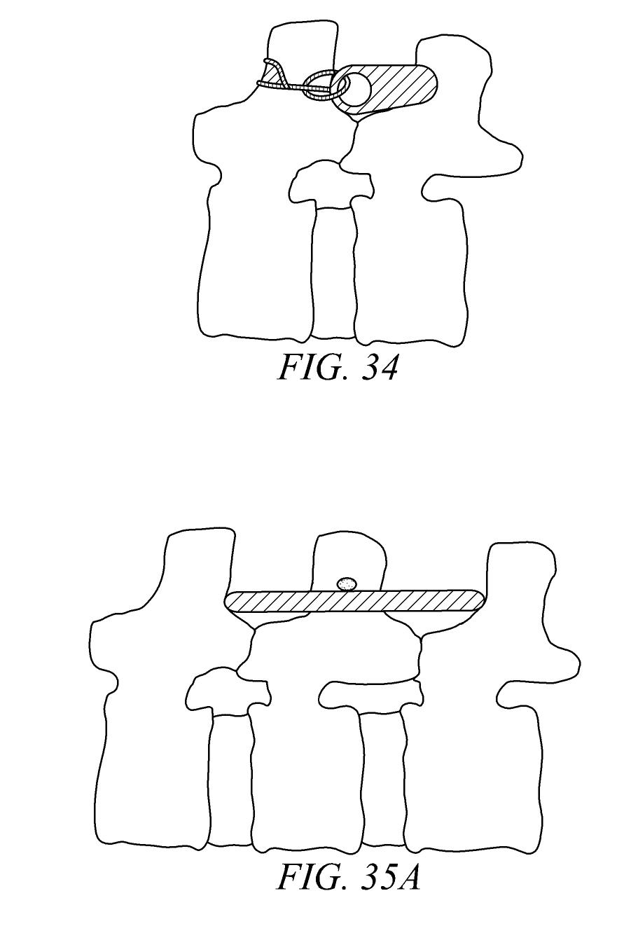

FIG. 34 is a lateral view of the spine and an alternative embodiment of the invention shown in FIG. 12A. The strap that surrounds the SPS is widened along the cranial aspect of the SP.

FIG. 35A is a lateral view of the spine and an alternative, multilevel embodiment of the invention related to that shown in FIG. 20B. The device distracts two adjacent levels of the spine. Allograft bone embodiments of the device could be treated to prevent fusion to the SP of the cranial and caudal vertebrae. For example, the cranial and caudal aspects of the device could be covered with bone wax, polymer, or other substance that inhibits bone growth to the device. The ends of the device could be constructed of only cortical bone. The center of the device is designed to fuse to the posterior elements of the intermediate vertebra. The center portion of an allograft bone device could include cortical and cancellous bone. Bone-growth-promoting substances could be placed between the device and the posterior elements of the intermediate vertebra. The posterior elements of the intermediate vertebra could be decorticated to facilitate fusion. The posterior elements of the vertebrae caudal and cranial to the device would not be decorticated.

FIG. 35B is a dorsal view of the spine and the embodiment of the invention shown in FIG. 35A. The device distracts two levels of the spine. The device preferably allows spinal flexion, but limits spinal extension at both levels of the spine.

FIG. 35C is a dorsal view of an alternative embodiment of the invention related to that shown in FIG. 20B. The two-component device is snapped together around a SP. The device may be held together through components 3540, 3542 that plastically deform when they area assembled. Alternatively the components could be made of a shape memory material such as Nitinol. FIG. 35D is a dorsal view of the spine and a three-level embodiment of the device shown in FIG. 35B.

FIG. 36A is a dorsal, exploded view of the spine and an alternative embodiment of the invention related to that shown in FIG. 35B. The device has a slot 3602 that accommodates more than one SP. FIG. 36B is dorsal view of the spine and the embodiment of the invention shown in FIG. 36A, wherein a cross member 3604 has been fastened to the device. The cross member 3604 fits between two adjacent spinous processes.

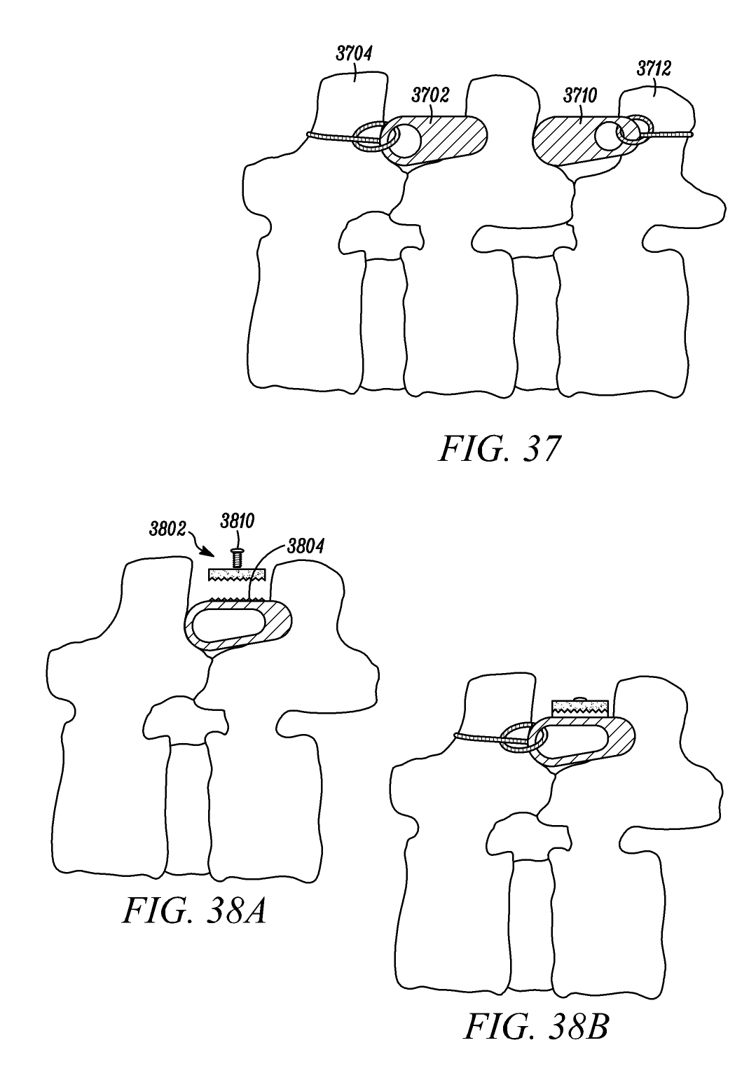

FIG. 37 is a lateral view of the spine and an alternative embodiment of the invention related to that shown in FIG. 33. A SPS 3702 has been connected to the caudal aspect of the cranial vertebra 3704 and a SPS 3710 has been connected to the cranial aspect of the caudal vertebra 3712 in a two-level construct. The cranial SPS is preferably fused to the cranial vertebra and the caudal SPS is preferable fused to the caudal vertebra. The arrangement keeps each SPS from impinging on the strap of an adjacent SPS.

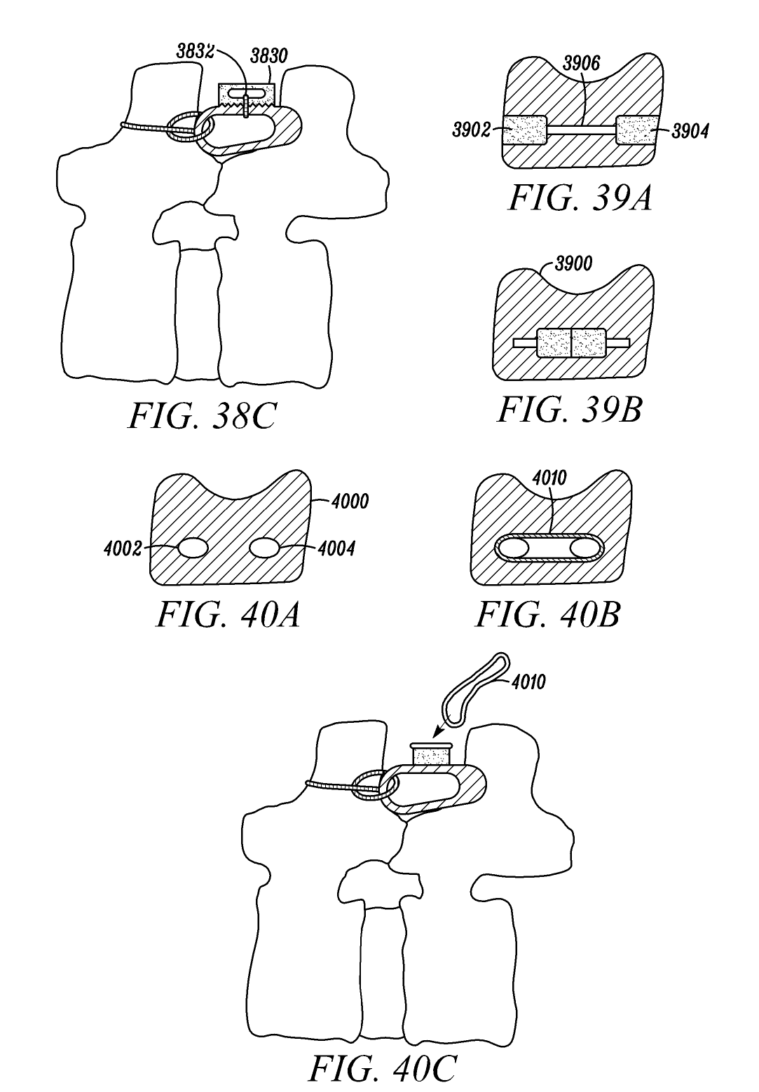

FIG. 38A is an exploded lateral view of spine and an alternative embodiment of the invention related to that shown in FIG. 12A. A component 3802 is attached to the dorsal aspect of the SPS. The two components 3802, 3804 have teeth along their mating surfaces. The teeth interdigitate to prevent movement of one component relative to the other component. A screw 3810 is used to connect the two components. The dorsal component 3810 helps prevent the SPS 3804 from rotating about the coronal axis of the spine. Rotation of the SPS about the coronal axis of the spine could reduce the distraction of the vertebrae. FIG. 38B is a lateral view of the spine and the embodiment of the invention shown in FIG. 38A. The assembled device has been attached to the SP using the technique taught with reference to FIG. 12A.

FIG. 38C is a lateral view of the spine and an alternative embodiment of the invention including a dorsal component 3830 attached to the SPS with a cable 3832. The dorsal component has a chamber. Bone or bone-growth promoting substances may be added to the chambers in both components of the device. Holes may pass between the chambers of both components.

FIG. 39A is a dorsal view of an alternative embodiment of the invention with components 3902, 3904 seen on the left and right sides of the device which slide along a slot 3906 formed across the dorsal surface of the SPS. The lateral locations of the dorsal components prevent the dorsal components from impinging against the spinous processes during rotation of the device. As taught in reference to FIG. 28C, rotation of the SPS in the coronal axis of the spine cams open the interspace.

FIG. 39B is a dorsal view of the embodiment of the invention shown in FIG. 39A. The components 3902, 3904 on the dorsal aspect of the device have been moved to the center of the device 3900. The dorsal components may be reversibly connected together. The components could snap together via portions of the components that plastically deform. Alternatively, the components could fasten together using shape-memory materials. The dorsal components are snapped together after the SPS is rotated to cam open the interspace. The connected dorsal components strike the spinous processes if the SPS is rotated after the components are connected. The configuration of the device prevents loss of distraction as the SPS is unable to rotate from the “cam” position.

FIG. 40A is a dorsal view of an alternative embodiment of the invention which has two projections 4002, 4004 on the dorsal aspect of the device 4000. FIG. 40B is a dorsal view of the embodiment of the invention shown in FIG. 40A. A cord 4010 has been wrapped around the projections on the dorsal aspect of the device. The cord is preferably an elastic band. FIG. 40C is a lateral view of the spine and the embodiment of the device shown in FIG. 40B. The band 4010 controls rotation of the device about the coronal axis of the spine. The band strikes the SP if the SPS is rotated about the coronal axis of the spine. The SPS may be rotated about the coronal aspect of the spine before the band is added to the device. The band is added to the device after the SPS is rotated into place.

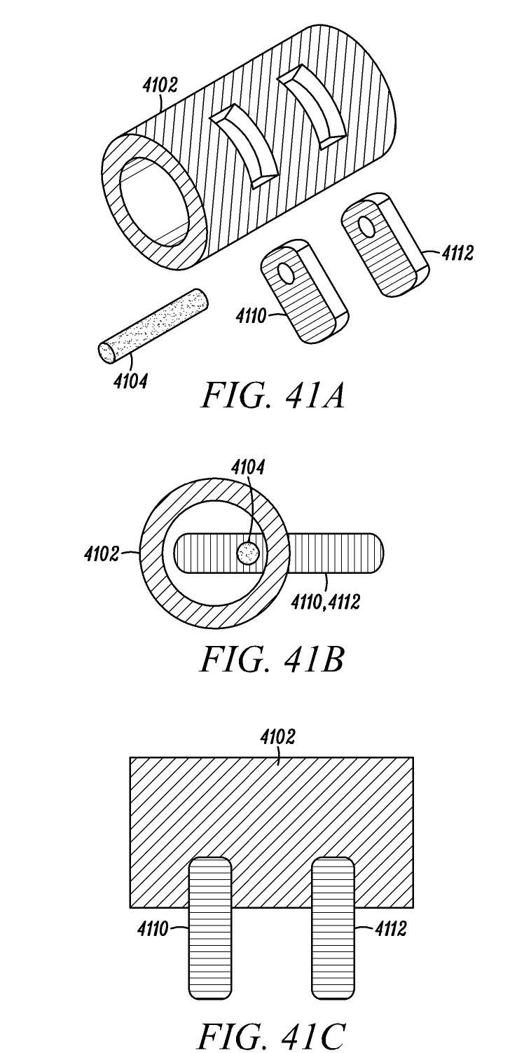

FIG. 41A is an exploded oblique view of an alternative embodiment of the invention may of allograft bone. The large cylinder 4102 could be made from the shaft of a long bone. The tibia, humerus, femur, radius, ulna, fibula, metatarsal; metacarpal, rib, pelvic bone, phalanges or other bones may be used to construct the device.

FIG. 41B is a lateral view of the embodiment of the device shown in FIG. 41A. A bone dowel 4104 has been placed through holes in the bone components 4110, 4112 that project from the cylinder shaped bone. The bone dowel holds the assembled bone SPS together. FIG. 41C is a dorsal view of the embodiment of the invention shown in FIG. 41B.

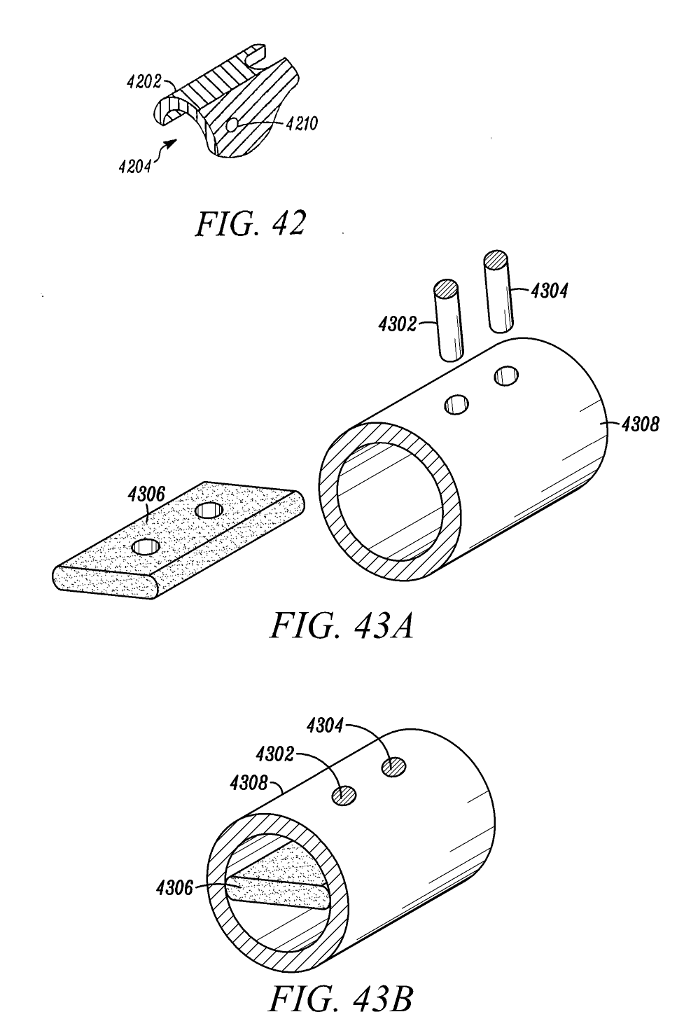

FIG. 42 is an oblique view of an alternative embodiment of the invention machined from the shaft of a single long bone. The ventral aspect 4204 of the SPS 4202 is open. The large opening on the ventral aspect of the SPS prevents the SPS from protruding into the spinal canal. Holes such as 4210 are drilled into the sides of the device. The holes can be used attach the SPS to the spine with suture or cables.

FIG. 43A is an exploded oblique view of an alternative embodiment of the invention similar to that shown in FIG. 41A. FIG. 43B is an oblique view of the device shown in FIG. 43B. Bone dowels 4302, 4304 are used to hold a rectangular or trapezoid shaped bone piece 4306 within a cylinder shaped bone 4308. The central bone component acts as a beam or column to strength the cylindrical bone.

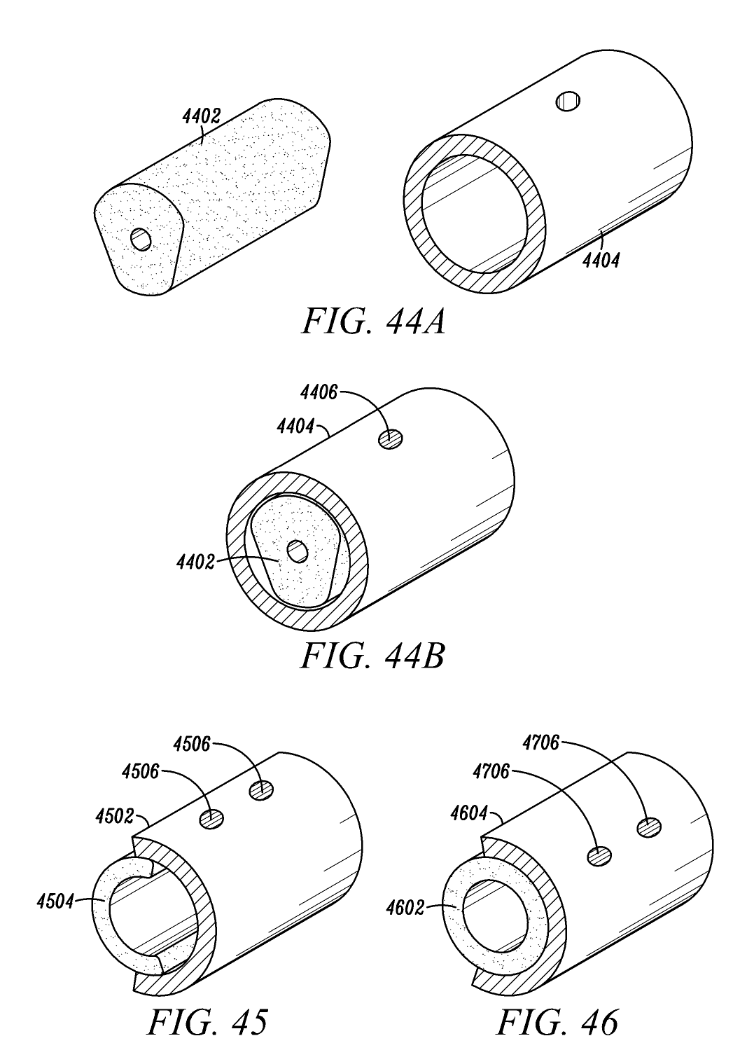

FIG. 44A is an exploded oblique view of an alternative embodiment of the invention similar to that shown in FIG. 43A. FIG. 44B is an oblique view of the SPS shown in FIG. 44A. A smaller bone 4402 is placed inside a larger bone 4404. The bones are held together with a bone dowel, screw, nail, staple, or other component 4406. For example, a portion of the shaft of a metatarsal bone could be placed inside a portion of the humerus.

FIG. 45 is an oblique view of an alternative embodiment of the invention shown in FIG. 44B. The device is manufactured by assembling the shafts of two bones 4502, 4504 that have been split along their longitudinal axes. The bones may be held together by bone dowels or other components 4506. The radius of one side of the assembled SPS is larger than the radius of the other side of the SPS.

FIG. 46 is an oblique view of an alternative embodiment wherein the shaft of a first bone 4602 has been placed into a portion of the shaft 4604 of a second bone. The larger bone has been split along its longitudinal axis. The smaller one projects through the opening in the larger bone. The assembled SPS can be held together with bone dowels or other fastening mechanism 4706.

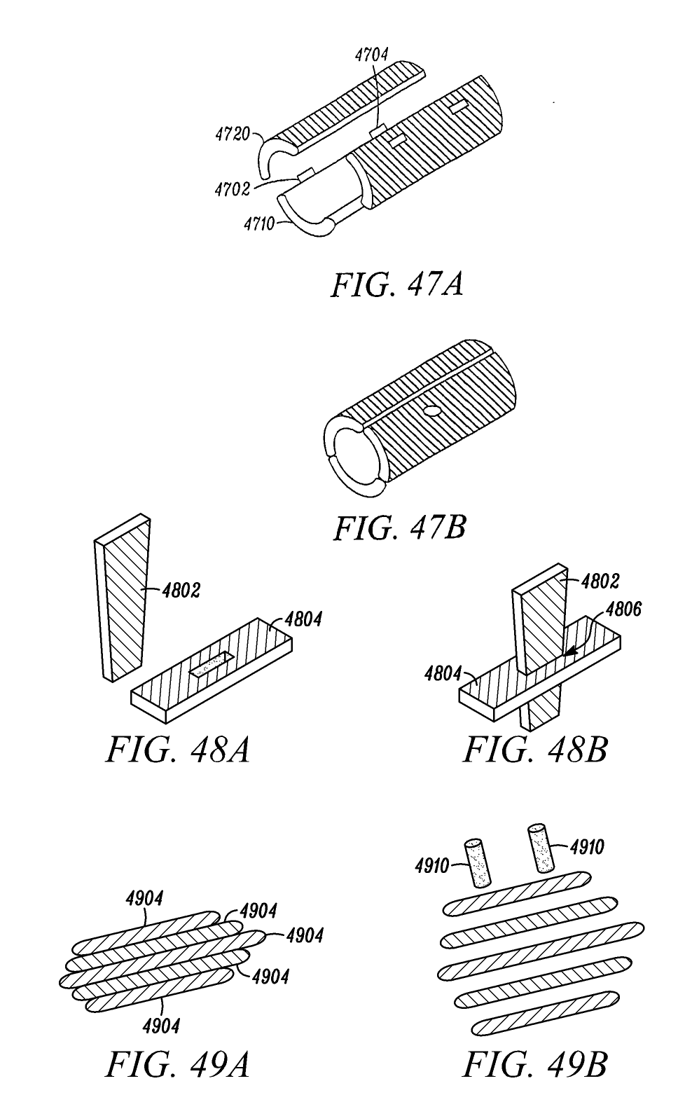

FIG. 47A is an exploded, oblique view of an alternative embodiment of the invention. FIG. 47B is an oblique view of the embodiment of the invention shown in FIG. 47A. Projections 4702, 4704 from one bone component 4710 fit into slots (not visible) in a second bone component 4720. Two or three pieces of bone are used to assemble the completed device, as shown in FIG. 47B. Other shapes of the assembled SPS can be manufactured by assembling more than three bones.

FIG. 48A is an exploded oblique view of an alternative embodiment of the invention wherein a first bone component 4802 is inserted into a slot of a second bone component 4804. FIG. 48B is an oblique view of the embodiment of the SPS shown in FIG. 48A. The bone components may be held together with bone pins 4806 on either slide of the slot within one of the bones. The shape of the SPS manufactured from two bones may be varied by changing the size of the bone components or the location and/or size of the slots within one of the components. Alternatively, the device could be manufactured with more than two bone components. For example a first bone component could be manufactured with two slots to receive two other bone components. A composite device could be constructed with bone and one or more other materials. For example, the device could be assembled from components made of bone and components made of PEEK.

FIG. 49A is lateral view of an alternative embodiment of the invention related to that shown in FIG. 10A. FIG. 49B is an exploded lateral view of the embodiment of the device shown in FIG. 49A. The device is assembled from pieces of bone 4904 that are stacked, machined, and pinned together. The pieces of bone are preferably pinned together with other pieces of bone 4910. FIG. 49C is a lateral view of an alternative embodiment of the invention similar to that shown in FIG. 49A. The pieces of bone have teeth 4920 on the dorsal and ventral surfaces where the pieces of bone contact with one another. The teeth interdigitate to improve the strength of the assembled bone SPS device.

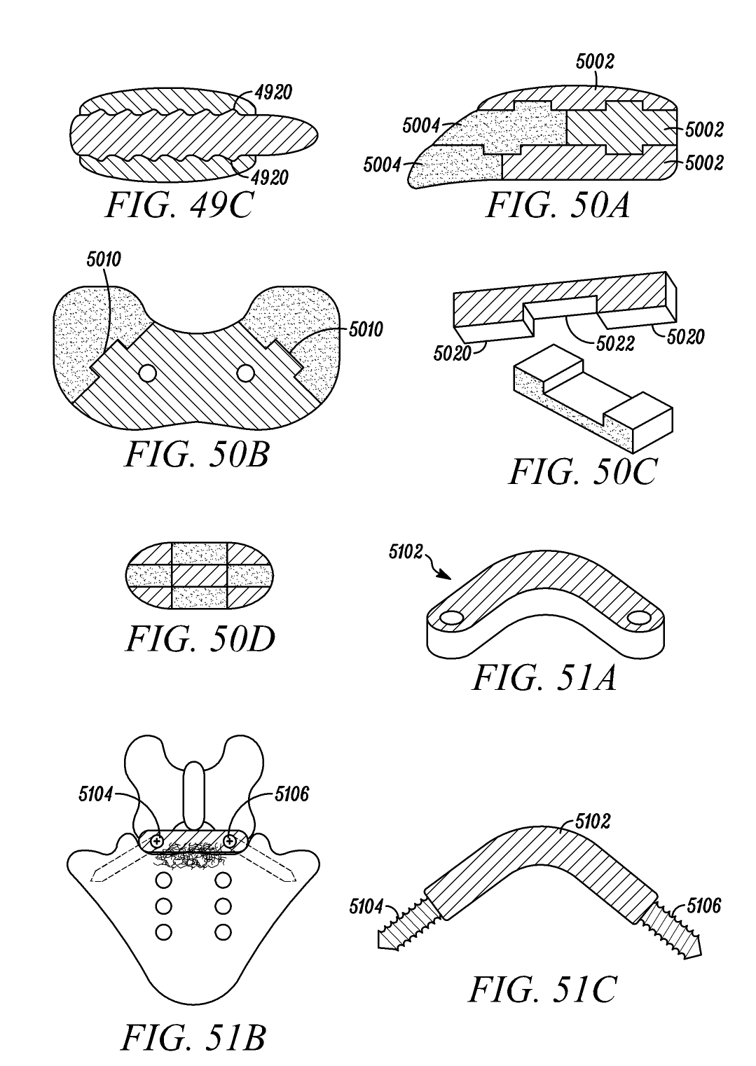

FIG. 50A is lateral view of an alternative embodiment related to the device shown in FIG. 49C which is assembled from multiple pieces of bone. The device may be constructed from machined pieces of cortical bone 5002 and pieces of cancellous bone 5004. Cortical bone is used to enable the device to receive loads from the vertebrae, whereas cancellous bone is used to facilitate fusion of the device to a single vertebra.

FIG. 50B is a dorsal view of the embodiment of the invention wherein the bone components are pinned together. Projections 5010 from bones fit into recesses in other bone components.

FIG. 50C is an oblique view of bones shaped to be connected in an alternative method than used in the device shown in FIG. 50A. Rectangular projections 5020 and slots 5022 are machined into the bones.

FIG. 50D is a lateral view of an alternative embodiment which has been constructed by assembling bones shaped like the bones in FIG. 50C. The pieces of bone may be assembled much like the pieces of wood are assembled in Jenga puzzles. The assembled bones could be pinned to hold the bones together.

FIG. 51A is an oblique view of an alternative embodiment of the invention related to that shown in FIG. 10A. FIG. 51B is a dorsal view of the spine and the embodiment of the invention shown in FIG. 51A. The V- or U-shaped device 5102 is designed to fit between the SP of the L5 vertebra and the sacrum. Screws 5104, 5106 connect the device to the sacrum. Bone-growth promoting material is placed over or in the device. Bone-growth material is also preferably placed on to the sacrum. The device is designed to fuse to the sacrum. The device may be made of bone, metal, ceramic, polymers, or other material. FIG. 51C is a caudal view of the embodiment of the device shown in FIG. 51A. Screws 5104, 5106 may be seen within the device. The screws may course in different directions. For example, the screws may converge. The device may be used in other levels of the spine. The screws may be placed into the pedicles of the vertebrae.

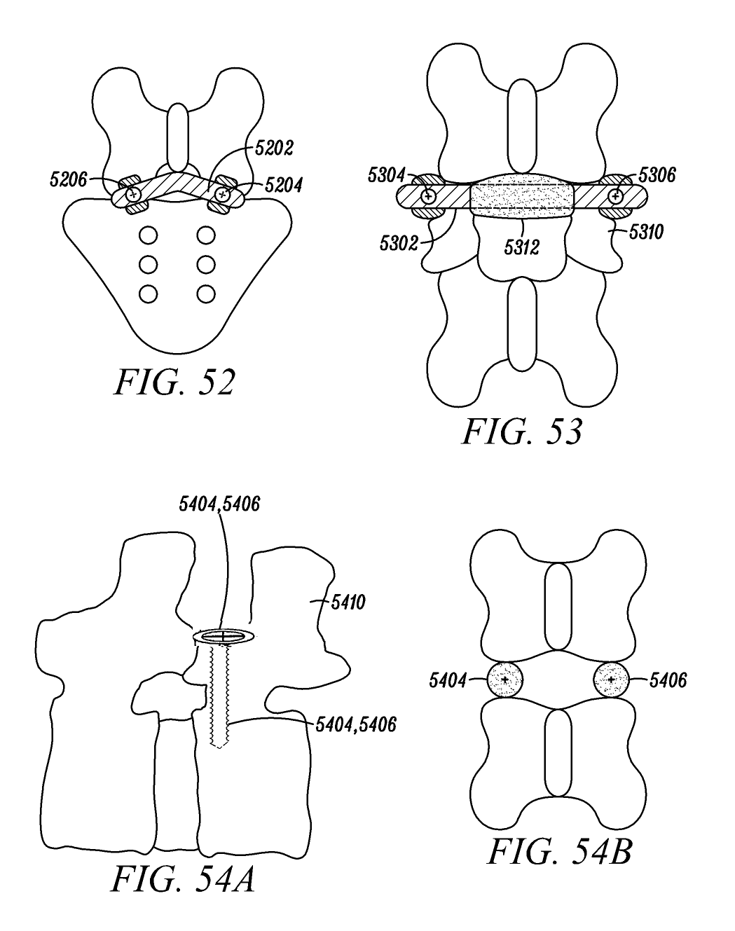

FIG. 52 is dorsal view of the spine and an alternative embodiment of the invention 5202 similar to that shown in FIG. 51B, which is connected to screws 5204, 5206 placed into the pedicles of the vertebra. The device may be made of metal, bone, ceramic, or polymers. The device may be fused to one of the vertebrae. Alternatively, the device may be used without promoting fusion to either vertebra.

FIG. 53 is a dorsal view of the spine and a version of the invention shown in FIG. 52. The device 5302 has been connected to screws 5304, 5306 placed into the pedicles of one of vertebrae 5310. The device is designed for use in patients who have undergone removal of one or more spinous processes. A “bumper” component 5312 has been placed over a component that courses from one pedicle screw to the other pedicle screw. The various components may be made of a polymer, metal, or bone.

FIG. 54A is a lateral view of the spine and an alternative embodiment of the invention shown in FIG. 53. Screws 5404, 5406 are placed into the pedicles of vertebrae 5410. The screws are placed through portions of the superior facets of the caudal vertebra. The inferior facets of the cranial vertebra impinge against the screws. The screws are placed after the spine is flexed. The screws allow spinal flexion but limit spinal extension.

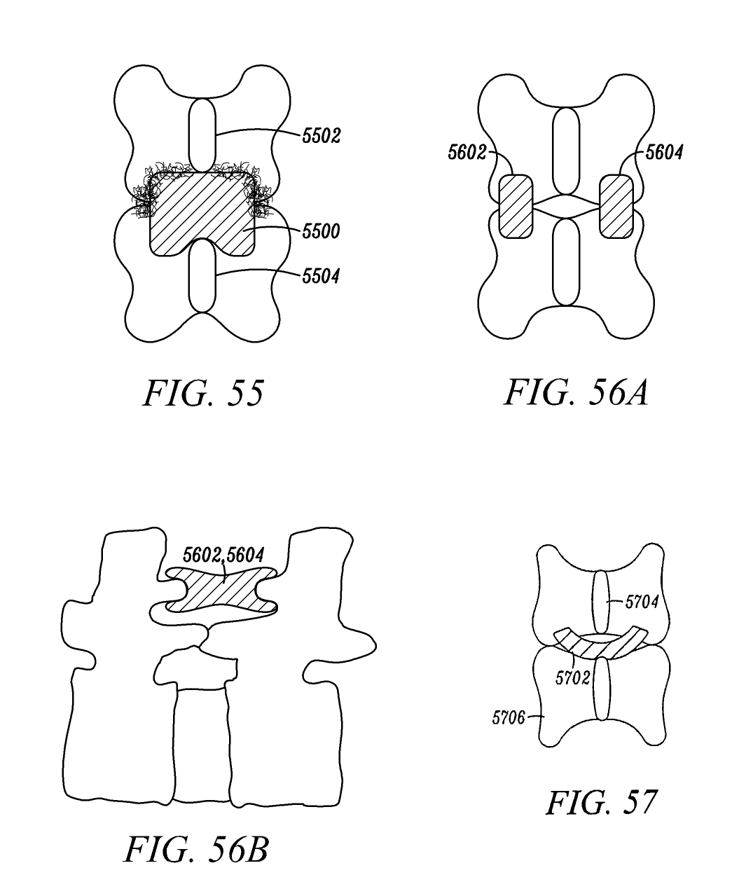

FIG. 55 is a dorsal view of the spine and an embodiment of the invention 5500 shown in FIG. 10A having been placed between the spinous processes 5502, 5504 of two vertebrae. The bone growth material extends into the facet joints between the two vertebrae. The bone growth material and the subsequent fusion mass cooperate with the SPS device to limit spinal extension.

FIG. 56A is a dorsal view of the spine and an alternative embodiment of the invention wherein paired devices 5602, 5604 are placed along the left and right sides of the dorsal aspect of the vertebrae. FIG. 56B is a lateral view of the spine and the embodiment of the invention shown in FIG. 56A. The devices fit over the caudal aspect of the lamina of the cranial vertebra and the cranial aspect of the lamina of the caudal vertebra.

FIG. 57 is a dorsal view of the spine and an alternative embodiment of the invention 5702 adapted to fit over the caudal aspect of the lamina 5704 of the cranial vertebra and the SP and/or lamina of the caudal vertebra 5706. The device distracts the vertebra and limits extension of the spine.

FIG. 58 is a lateral view of the spine and an alternative embodiment of the invention related to that shown in FIG. 10A. The dorsal and ventral surfaces of the cranial end of the device have concavities 5802, 5804 to receive bone-growth-promoting material (not shown).

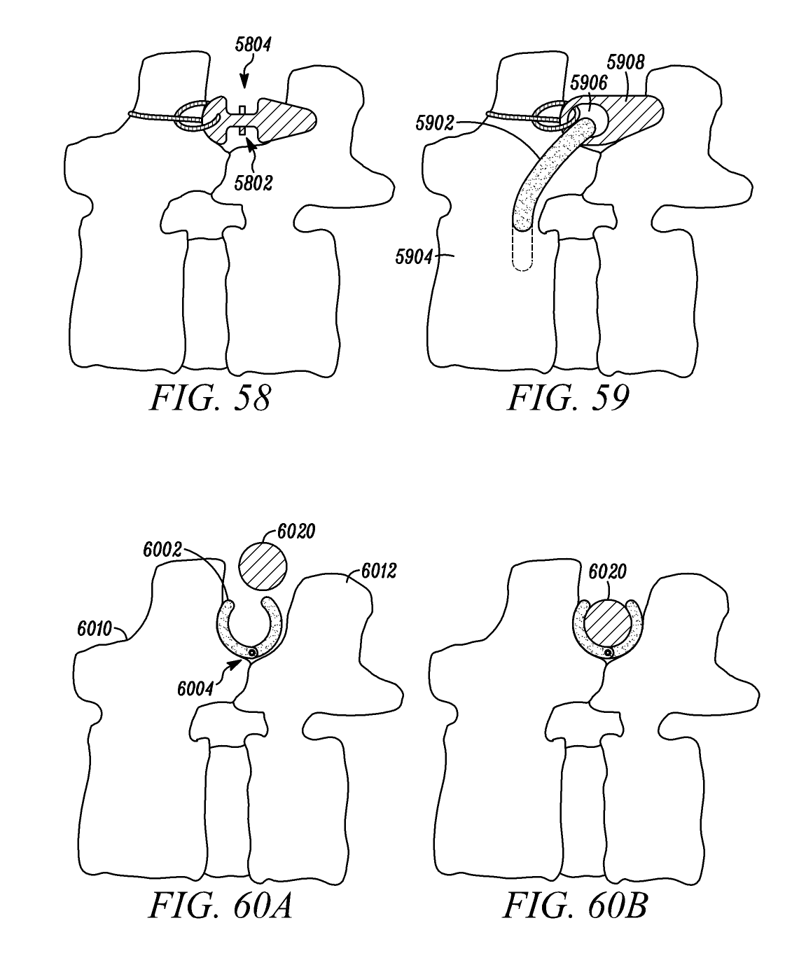

FIG. 59 is a lateral view of the spine and the embodiment of the invention related to that shown in FIG. 10A. A tube 5902 passes from a hole in the pedicle 5904 of the vertebra to the concavity 5906 of the device 5908. The tube 5902 facilitates the migration of cells from the body or pedicle of the vertebra to the bone growth promoting material. Alternatively, cells obtained from aspirating the vertebra or other bone may be added to the bone growth material in the device.

FIG. 60A is a lateral view of the spine and an exploded, lateral view of an alternative embodiment of the invention including a semi-cylindrical component 6002 with a hinge joint 6004 is placed between the spinous processes of two adjacent vertebrae 6010, 6012. FIG. 60B is a lateral view of the spine and the embodiment of the invention shown in FIG. 60A. A rod 6020 has been placed into the hinged semi-cylindrical component. The rod component expands the hinged component and distracts the spine. Spring-like properties of the hinged component or shape-memory properties of the components could be used to fasten the components.

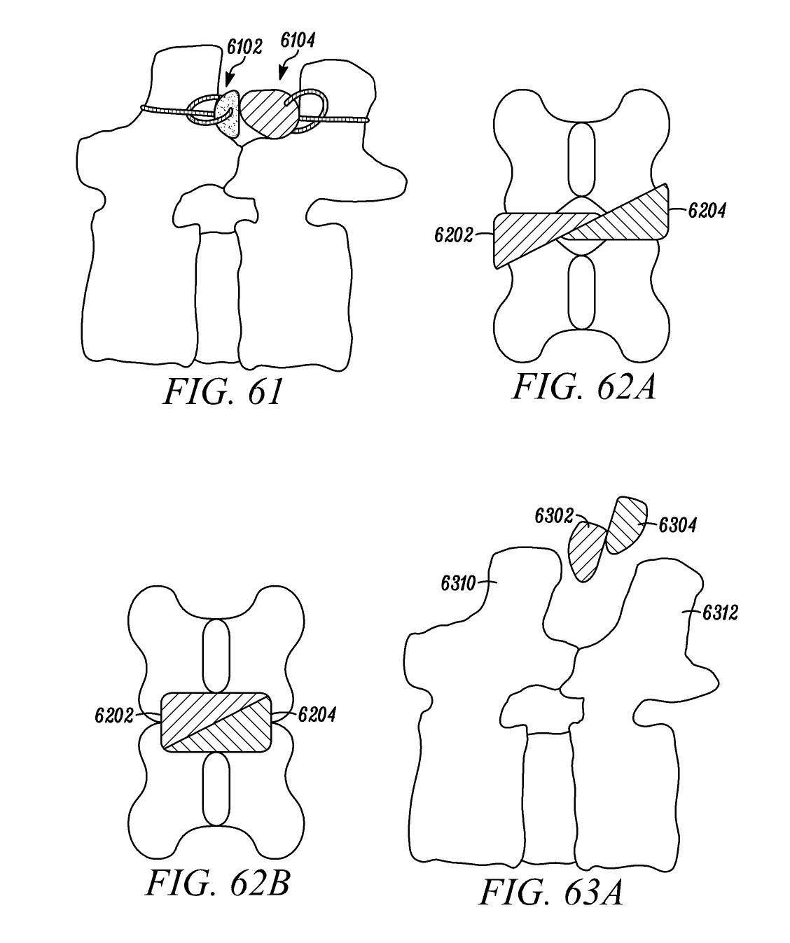

FIG. 61 is a lateral view of the spine and an alternative embodiment of the invention related to that shown in FIG. 10A. A component 6102 attached to one SP impinges against a component 6104 attached to a SP of an adjacent vertebra. The components may be attached to the spinous processes using the method taught in FIG. 12A. The components could be made of metal, polymers, ceramic, bone, fabric or combinations thereof.

FIG. 62A is a dorsal view of the spine and a further alternative embodiment of the invention related to that shown in FIG. 10C. Two wedge-shaped components 6202, 6204 are connected and inserted between the spinous processes of two adjacent vertebrae. FIG. 62B is a dorsal view of the spine and the embodiment of the invention shown in FIG. 62A. The wedge components 6202, 6204 have been urged together so as to increase the width of the device in the cranial-to-caudal direction, thereby distracting the vertebra. The components could be drawn together with screws. Alternatively, the components could be forced together with pliers. The components could be locked in the compressed position using screws or other fasteners, plastic deformation technology, or shape-memory technology.

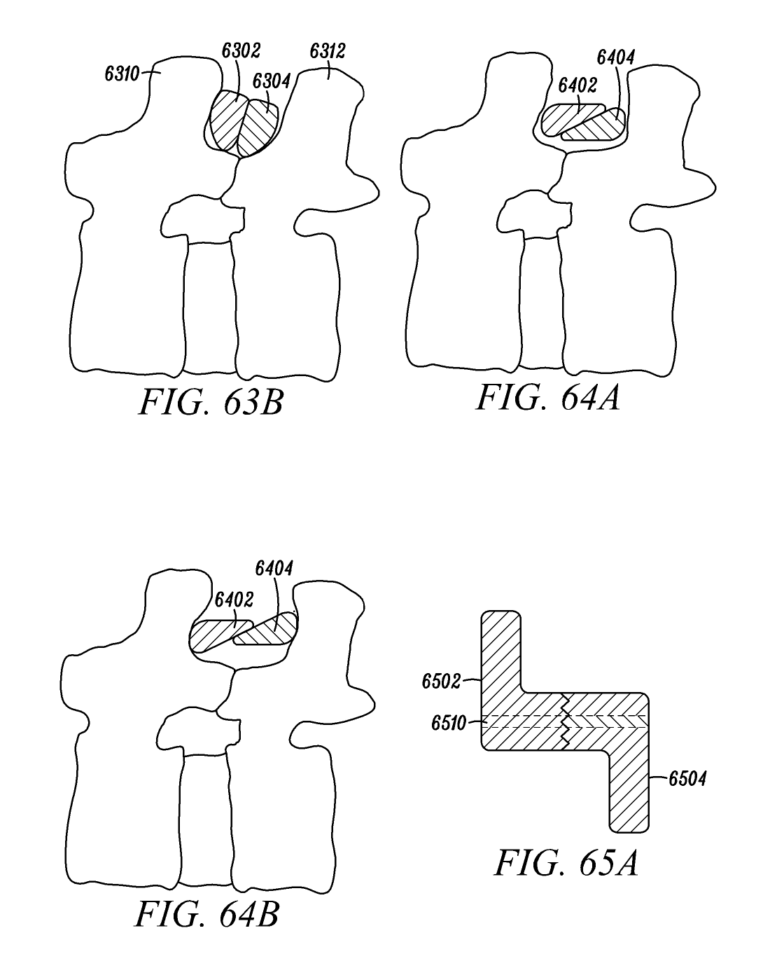

FIG. 63A is a lateral view of the spine and a variation of the embodiment of the invention shown in FIG. 62A. FIG. 63B is a lateral view of the spine and the embodiment of the device shown in FIG. 63A. Two components 6302, 6304 are compressed together after placing the device between the spinous processes 6310, 6312 of adjacent vertebrae. The device distracts the spine as the components are forced together. The components may be locked in their compressed position.

FIG. 64A is a lateral view of the spine and different configuration of the invention shown in FIG. 63B. FIG. 64B is a lateral view of the spine and the embodiment of the invention shown in FIG. 64A. The device distracts the spine as the components 6402, 6404 are forced apart. The components may be locked in their extended position.

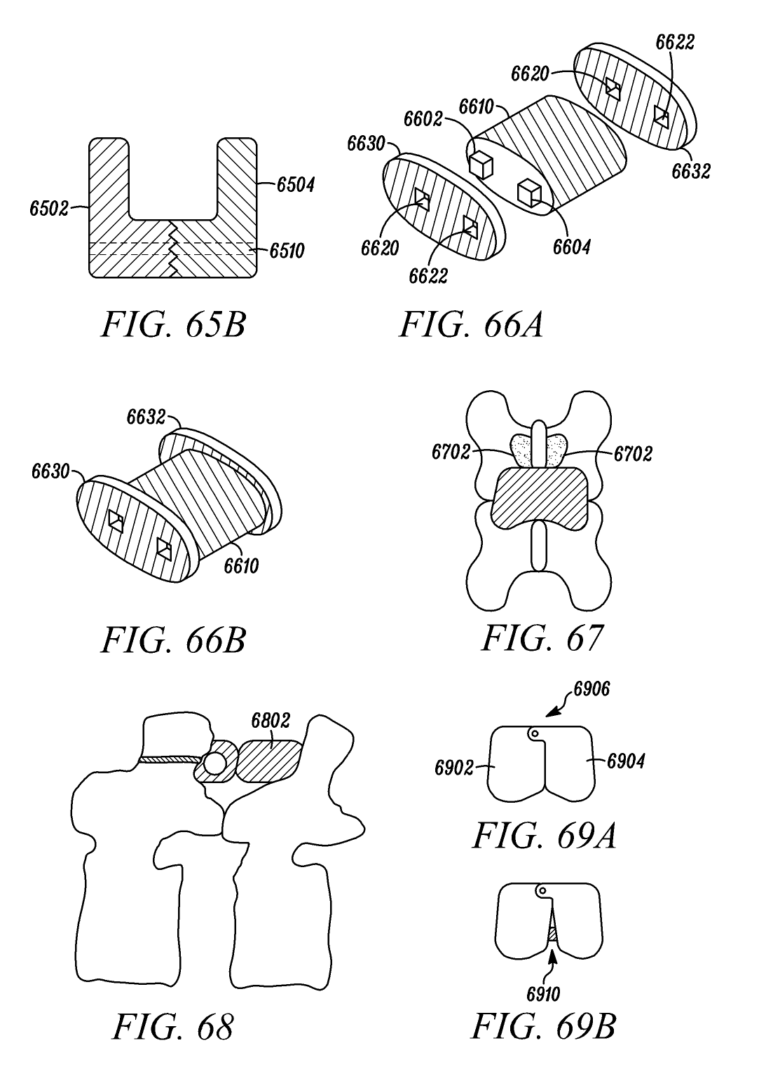

FIG. 65A is a dorsal view of an alternative embodiment of the invention related to that shown in FIG. 10A. FIG. 65B is a dorsal view of the embodiment of the device shown in FIG. 65A. The two components 6502, 6504 of the device articulate at the joint between the components. The positions of the components may be changed by rotating one component relative to the second component. A screw 6510 may be used to lock the components in a desired position.

FIG. 66A is an exploded oblique view of an alternative embodiment of the invention related to that shown in FIG. 41A. The device is preferably made of bone. FIG. 66B is an oblique view of the device shown in FIG. 66A. Projections 6602, 6604 from the central component 6610 are forced into holes 6620, 6622 in the lateral components 6630, 6632.

FIG. 67 is a dorsal view of the spine and an alternative embodiment of the invention related to that shown in FIG. 10A. The cranial aspect of the device 6702 is made of a resorbable material designed to resorb after the device fuses to one of the vertebrae.

FIG. 68 is a lateral view of the spine and an alternative embodiment of the device related to that shown in FIG. 12A. A compressible, resilient or elastic component 6802 is attached to the caudal end of the device. The component 6802 dampens loads across the device.

FIG. 69A is a dorsal view of an alternative embodiment of the invention. Components 6902, 6904 on the left and right side of the device are connected with a hinge joint 6906. The components are also connected with one or more elastic bands. Compression on the caudal end of the device hinge the two components open. The device dampens loads applied by the spinous processes. FIG. 69B is a dorsal view of the device shown in FIG. 69A. The device has been partially opened to show the bands 6910.

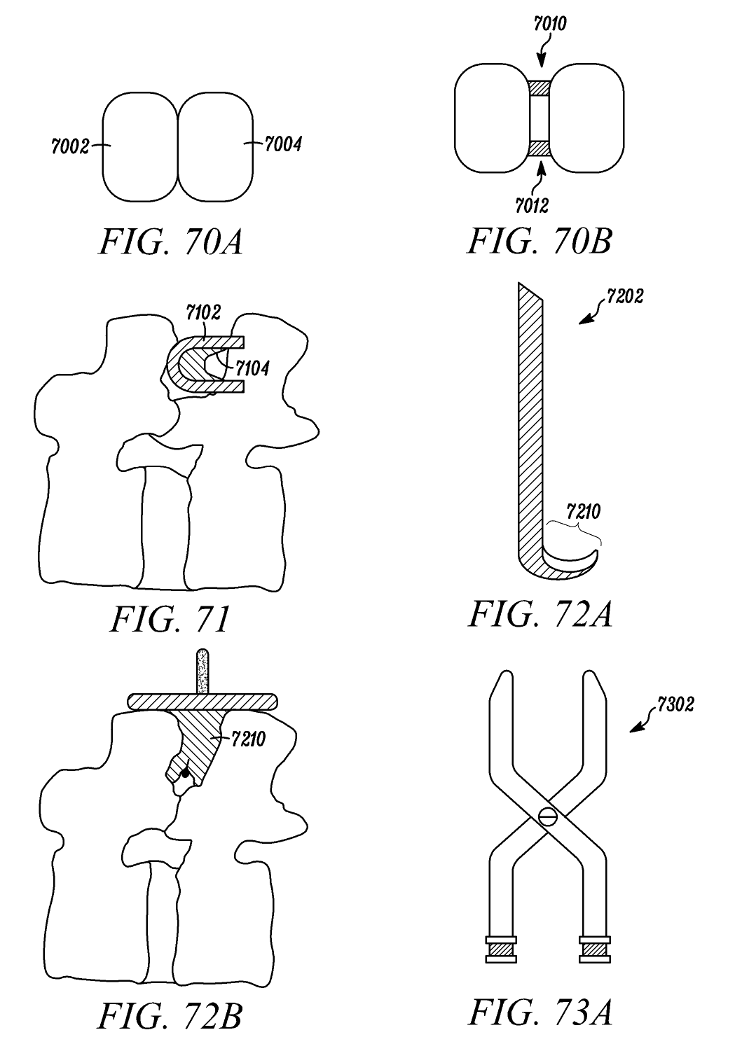

FIG. 70A is a dorsal view of an alternative embodiment of the invention similar to that shown in FIG. 69A. FIG. 70B is a dorsal view of the device shown in FIG. 70A. The components 7002, 7004 on the left and right sides of the device are connected with two or more elastic cords 7010, 7012. The device is shown in its opened position. The device may be opened by forces from the spinous process adjacent to the device. The device dampens loads applied by the spinous processes.

FIG. 71 is a lateral view of the spine and an alternative embodiment of the invention utilizing components preferably made from the shafts of bones. The bone components 7102, 7104 may be pinned in the configuration illustrated in the figure.

FIG. 72A is lateral view of a knife-like instrument 7202 that may be used to cut the ligaments between the spinous processes. The cutting surface of the knife is shown at 7210. FIG. 72B is a lateral view of the spine and the cutting tool shown in FIG. 72A. The device 7202 has partially severed the interspinous ligament 7210. The device cuts the ligament as it is pulled away from the spinal canal.

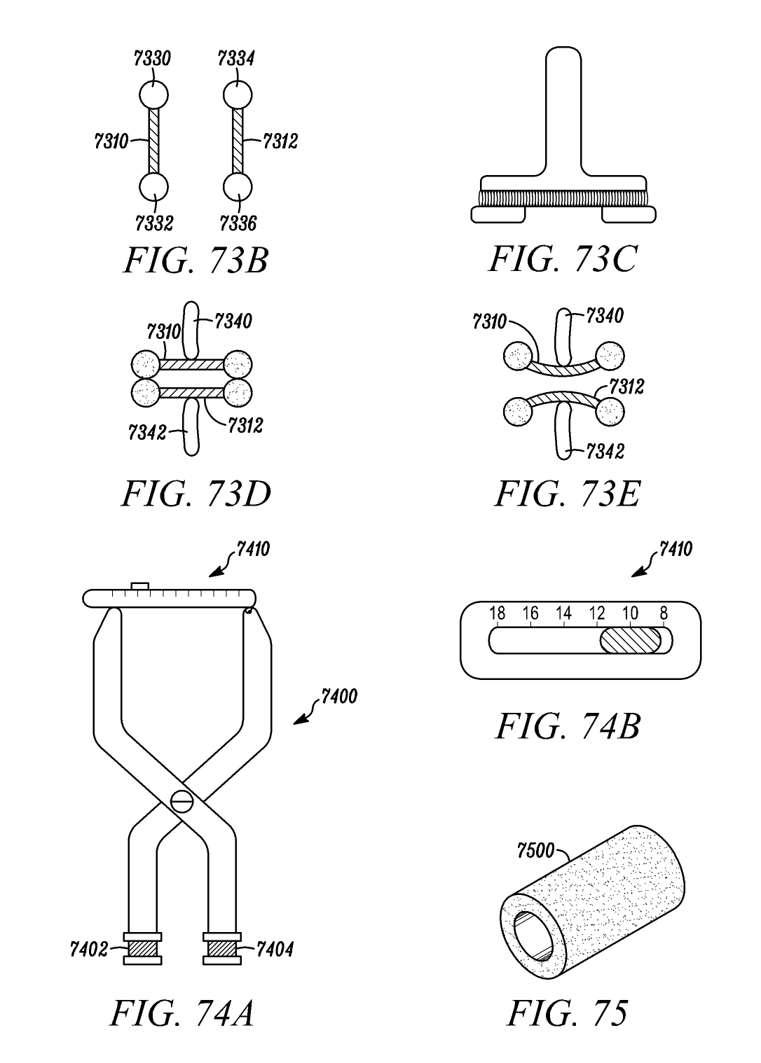

FIG. 73A is a lateral view of a tool 7302 used to distract the spinous processes. FIG. 73B is a view of the one end of the distracting tool shown in FIG. 73A. Fabric or elastic bands 7310, 7312 connect the tips 7330, 7332, 7334, 7336 of the tool.

FIG. 73C is a lateral view of the tool, and FIG. 73D is a view of the dorsal aspect of two adjacent spinous processes and the end of the tool shown in FIG. 73C. The fabric bands 7310, 7312 fit between the spinous processes 7340, 7342. The thin bands conform to the shape of the spinous processes while applying pressure over a large area. The flexibility and the size bands protect the spinous processes from injury during spinal distraction.

FIG. 73E is a dorsal view of two adjacent spinous processes and the tips of the tool shown in FIG. 73D. The drawing illustrates the tool distracting the spinous processes 7340, 7342. The handle of the tool may include a gauge (not shown) that measures the force applied to the tool or the distance the tips of the tools have opened. The method may include distracting the spinous processes a certain distance (for example, 5 mm), a certain percent (for example 20%) or until a certain amount of force is applied (for example, 20 inch/pounds).

FIG. 74A is a lateral view of a measuring tool 7400 having tips 7402, 7404 that are placed into the interspinous space. The tool may be used to distract the spinous processes and measure the distance between the spinous processes. The information may be used to determine the proper size of the device to be inserted between the spinous processes. FIG. 74B is a view of a gauge 7410 used on the handle of the instrument shown in FIG. 74A. The gauge suggests the proper size of the SPS device to insert between, the spinous processes.

FIG. 75 is an oblique view of a sleeve 7500 according to the invention that may be placed over the cables used in embodiments of the invention including that shown in FIG. 12A.

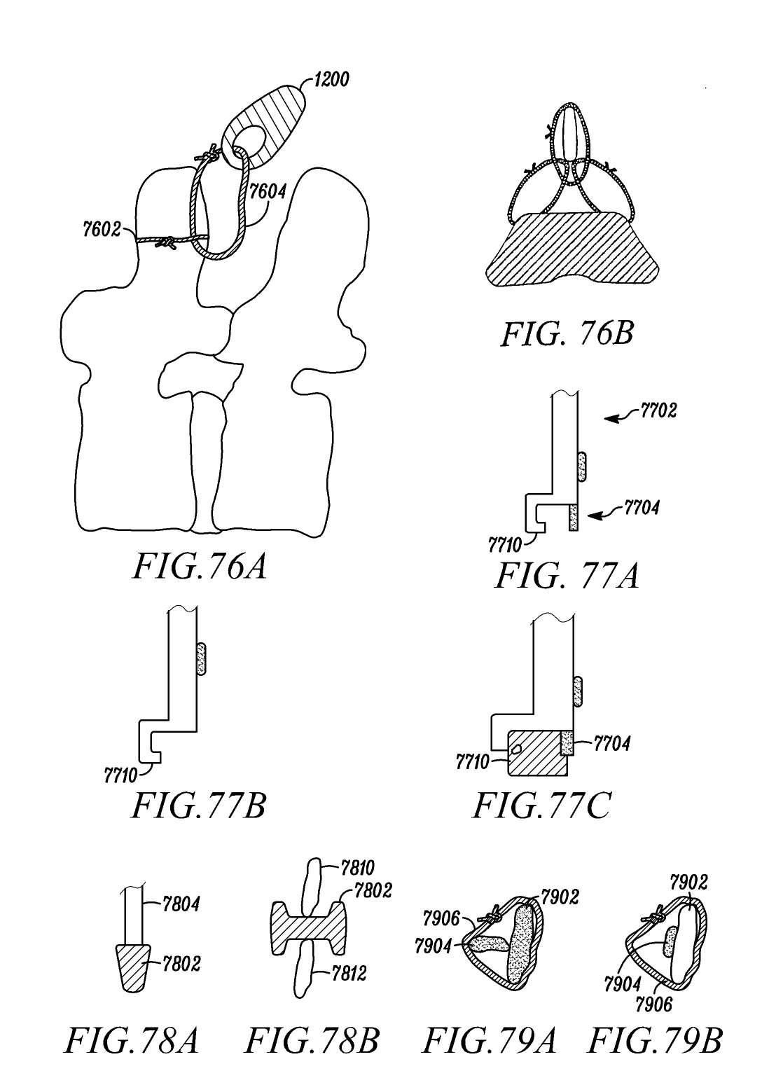

FIG. 76A is a lateral view of the spine and the embodiment of the invention shown in FIG. 12A. A cable 7602 has been looped around the SP of the cranial vertebra. The cable also surrounds cable 7604 loops that attach to the left and right sides of the device 1200. The cables on the left and right sides of the device are tightened after the device is placed between the spinous processes. FIG. 76B is a dorsal view of the spine and the embodiment of the invention shown in FIG. 76A.

FIG. 77A is a lateral view of the tip of an instrument 7702 that may be used to hold the SPS device. FIG. 77B is a lateral view of the tip of the instrument shown in FIG. 77A. A retractable member 7704 is shown in its retracted position. FIG. 77C is a lateral view of the tip of the tool shown in FIG. 77A and a SPS device. A projection 7710 from the tool is placed into a hole on the lateral side of the SPS device. The retractable arm 7704 passes over the other side of the SPS, thus holding the SPS device in the tool.

FIG. 78A is a lateral view of the tip 7802 of a distractor tool 7804 according to the invention. FIG. 78B is a dorsal view of the tips of two spinous processes 7810, 7812 and the tip of the distractor tool shown in FIG. 78A. The wedge shaped distractor tool is forced between adjacent spinous processes to wedge the spinous processes open.

FIG. 79A is a dorsal view of the tip of a SP 7902, a cross section of an inventive tool 7904, and a cable 7906. The tool is used to prevent over tightening the lower cable in the embodiment of the invention shown in FIG. 12D. FIG. 79B is a dorsal view of the tip of a SP, the cross section of the tool shown in FIG. 79A and a cable. The tool has been rotated 90 degrees. Rotating the tool allows removal of the tool. Removing the tool provides sufficient slack in the cable to allow the SPS device to move away from the caudal vertebra.

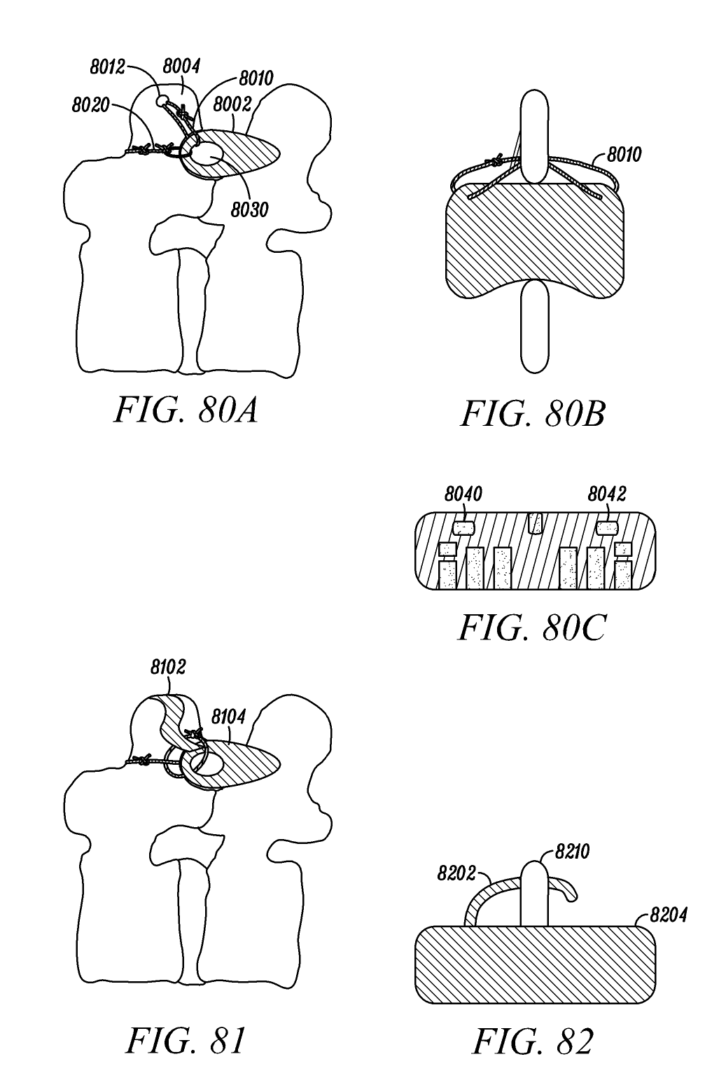

FIG. 80A is a lateral view of the spine and an alternative embodiment of the invention related to that shown in FIG. 12A. The SPS 8002 is attached to the dorsal portion of the SP 8004 of the cranial vertebra. A cable, cord, wire suture or other flexible member(s) 8010 pass through a hole 8012 in the SP. The flexible member “bridle” 8020 also attaches to the left and right sides of the SPS. For example, a cable could pass through hole(s) 8030 in the SPS. This dorsal cable and attachment mechanism prevents the SPS from migrating into the spinal canal. The invention may be particularly helpful in patients treated with unilateral or bilateral laminotomies and/or partial facetectomies. A portion of the caudal end of the lamina is removed during laminotomies. The medial portions of the facet area removed during partial facetectomies. Facetectomy and laminotomy enlarge the spinal canal. The invention helps prevent SPSs from falling into the enlarged opening into the spinal canal. A sleeve could be used to increase the surface area of the cable. The sleeve could fit over the cable where the cable passes through the hole in the SP. Alternatively, a grommet could be placed into the hole in the SP.

FIG. 80B is a dorsal view of the spine and the embodiment of the invention shown in FIG. 80A. The bridle cable 8010 passes from one side of the SPS, through a hole in the SP, to the other side of the SPS. The cables and methods illustrated in FIG. 12A were not shown to better illustrate the bridle cable. FIG. 80C is a cranial view of the embodiment of the SPS shown in FIG. 80A. The circles 8040, 8042 on the left and right sides of the dorsal portion of the SPS are designed to accept the ends of the bridle cable.

FIG. 81 is a lateral view of the spine and an alternative embodiment of the invention wherein a portion 8102 of the SPS 8104 extends over the dorsal aspect of the SP. The dorsal aspect of the SP could be notched to help prevent the SPS from sliding off of the SP. Alternatively, the SPS may have a projection that extends over the SP of the caudal vertebra or the spinous processes of the cranial and the caudal vertebrae. Additional embodiments may use a harness, bridle, or mesh that extends from the left and right sides of the SPS and over one or more spinous processes. Alternatively, the invention could use a single member that extends from one side of the SPS to the SP. The unilateral embodiment of the invention is preferably placed on the side of the unilateral “hemi” laminotomy.

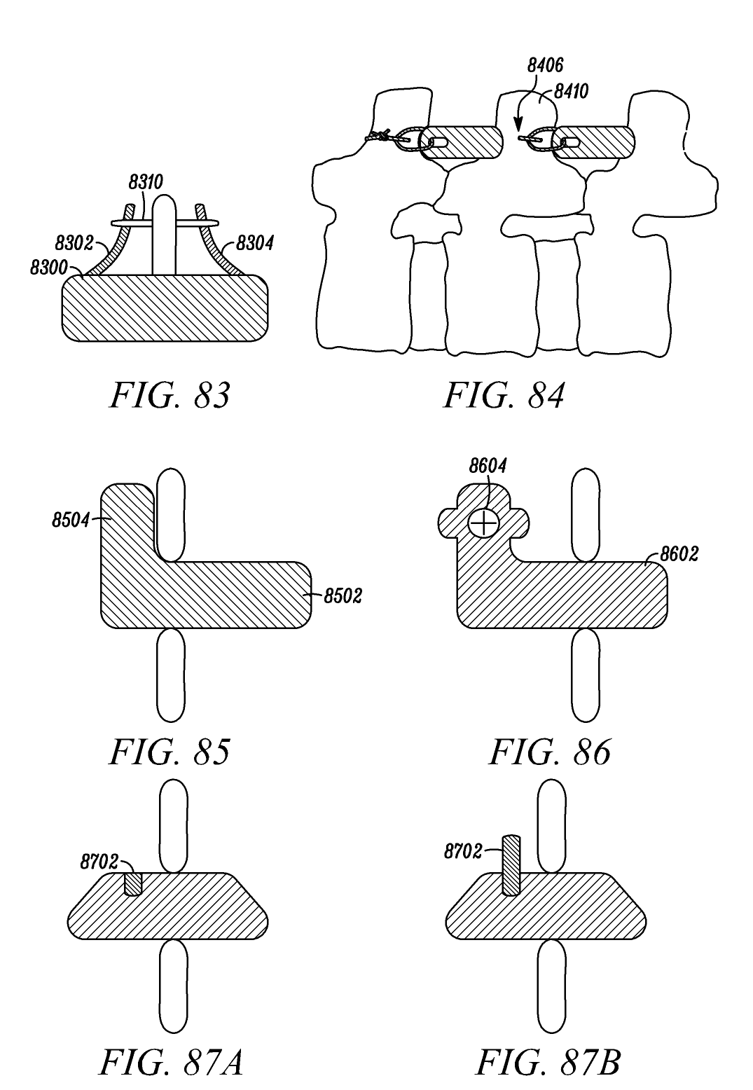

FIG. 82 is a caudal view of a SPS and an alternative embodiment of the invention related to that shown in FIG. 81. A projection including a hook 8202 from the SPS 8204 passes through a hole in the SP 8210.

FIG. 83 is a caudal view of an alternative embodiment of the invention similar to that shown in FIG. 82. Cables or other members 8302, 8304 pass from the sides of the SPS 8300 to a member 8310 that was placed into a hole in the SP. The cables or other members that pass through the SP could be made of bone, metal, ceramic, plastic, or other material. The component 8310 that passes through a hole in the SP preferably is made of a material that allows the patient's bone to grow into the component.

FIG. 84 is a lateral view of the spine and a variation of the embodiment of the invention shown in FIG. 37. The cable that connects the caudal SPS to the SP of the intermediate vertebra passes through a hole 8406 in the SP of the intermediate vertebra 8410. The hole in the SP is preferably located in the center of the SP. Alternatively, the cable could pass through another portion of the intermediate vertebra. For example, the cable could pass through holes in the lamina.

FIG. 85 is a dorsal view of the spine and an alternative embodiment of the invention related to that shown in FIG. 80A. The SPS 8502 has a projection 8504 from the cranial portion of the device. The projection extends over a portion of the lamina cranial to laminotomy defect. Projections could extend from the left and right sides of the SPS. A unilateral projection could extend from the SPS on the side of the laminectomy. Alternatively, a unilateral projection could extend from the SPS on the side contralateral to the laminectomy. FIG. 86 is a dorsal view of the spine and an alternative configuration of the invention shown in FIG. 85. The projection 8602 from the cranial portion of the SPS is connected to a screw 8604. The screw is preferably placed into on of the pedicles of the cranial vertebra.

FIG. 87A is a dorsal view of the spine and an alternative embodiment of the invention related to that shown in FIG. 85. The drawing illustrates a retractable projection member 8702 in its retracted position. FIG. 87B is a dorsal view of the spine and the embodiment of the SPS shown in FIG. 87A. The member 8702 is shown in its extended position. The projection member may locked in the extended position.

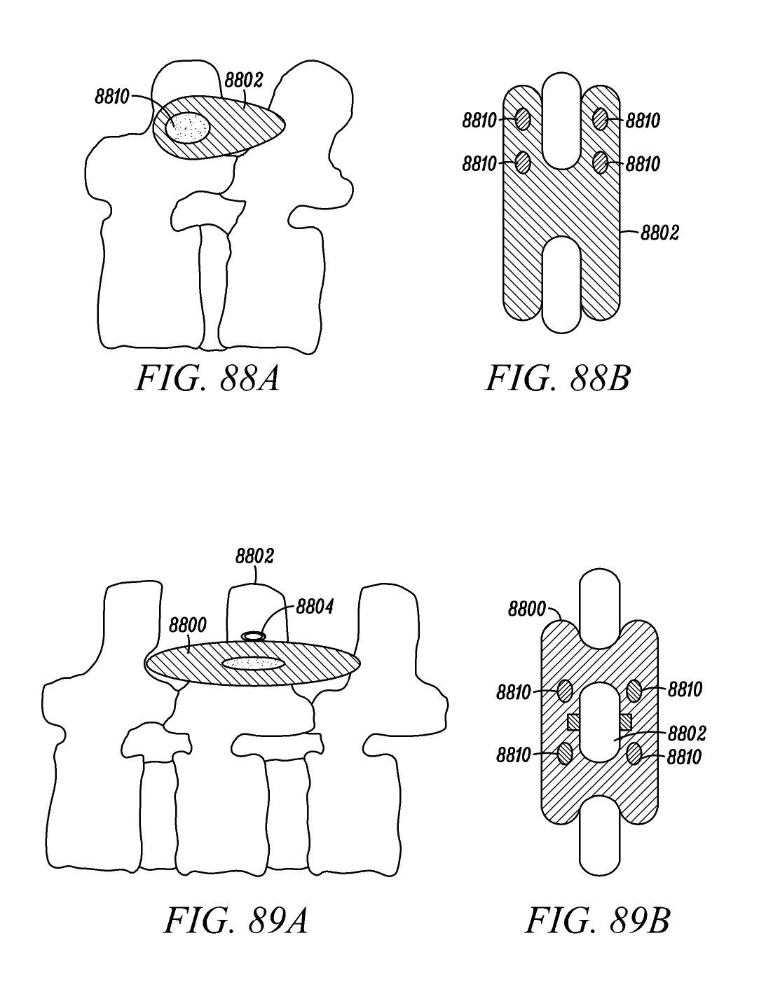

FIG. 88A is a lateral view of the spine an alternative embodiment of the invention related to that shown in FIG. 20A. The SPS device 8802 has two or more holes or chambers 8810 that may be filled with bone or bone growth promoting material. The device could be attached to the spine in the method taught in reference to FIG. 12A. As with most other embodiments described herein, the device may be constructed of bone, metal, polymer, ceramic, or other material. The circle with dots represents a chamber in the side of the device. FIG. 88B is a dorsal view of the spine and the embodiment of the invention shown in FIG. 88A.

FIG. 89A is a lateral view of the spine and an alternative embodiment of the invention related to that shown in FIG. 88A. The device 8800 fits over SP 8802 and distracts two sets of adjacent spinous processes. Pin 8804 may be used to hold the device in place, and holes/apertures 8810 may be provided for bone ingrowth. FIG. 89B is a dorsal view of the spine and the embodiment of the invention shown in FIG. 89A.

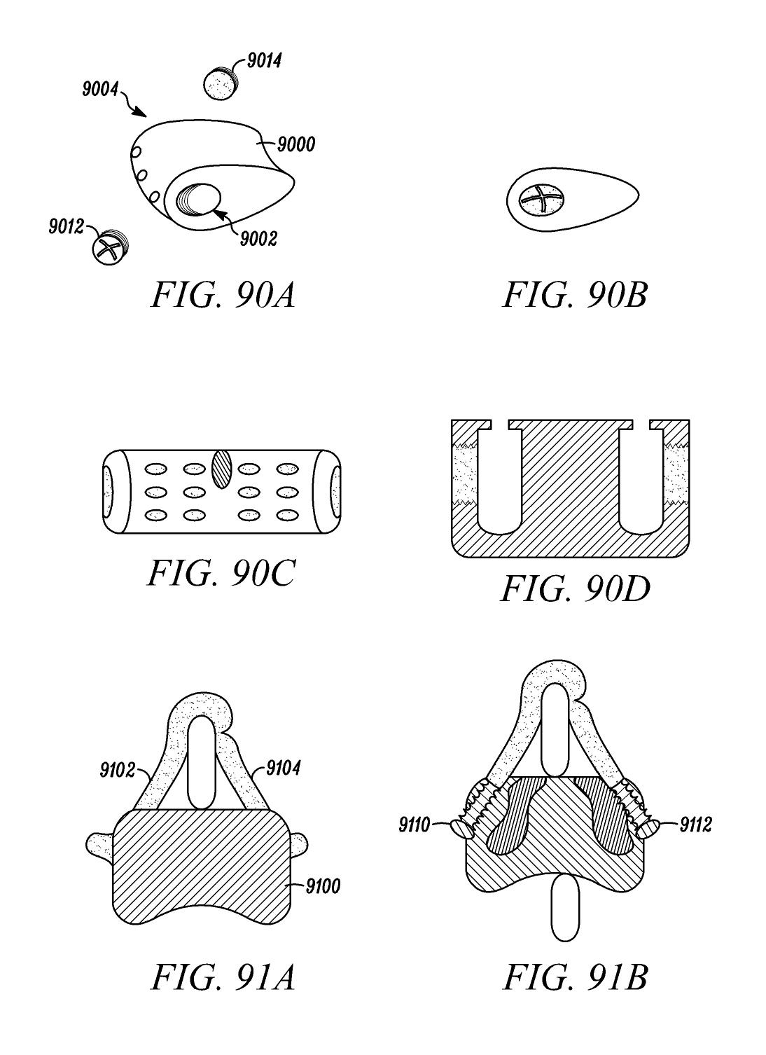

FIG. 90A is an exploded oblique view of an alternative embodiment of the invention related to that shown in FIG. 10A. Openings 9002, 9004 on the left and right side of the device 9000 may be optionally closed with additional components 9012, 9014. The additional components may be screwed into the openings in the sides of the device. Alternative mechanisms may be used to fasten the side components to the device. Bone or bone-growth promoting substances may be placed into the device before fastening the side components. Tether fastening components may be passed through the bone in-growth holes on the cranial portion of the device.

The bone in-growth holes are limited to the cranial portion of the assembled device. Alternatively, the in-growth holes may be limited to the caudal portion of the device, the ventral portion of the device, the dorsal portion of the device, or any combination of two, three, or more portions of the device. The invention may also include one component device that does not have holes on the left and/or the right sides of the device. FIG. 90B is a lateral view of the assembled device shown in FIG. 90A. FIG. 90C is an anterior view of the assembled device shown in FIG. 90B: FIG. 90D is coronal cross section of the assembled device shown in FIG. 90C.

FIG. 91A is a dorsal view of the spine and an alternative embodiment of the invention related to that shown in FIG. 12A. Components 9102, 9104 from one end of the device 9100 pass over the cranial end of the cranial SP or the caudal aspect of the SP caudal to the device. The components may be tightened to force the device against the SP or lamina. Forcing the device against the SP, or lamina, eliminates movement between the device and the posterior elements of the spine the device is attached to.

FIG. 91B is a coronal cross section of the spine and the embodiment of the device shown in FIG. 91A. The fixation components may be locked in the tightened position. For example, the fixation components may include nuts 9110, 9112 that is threaded onto the component. The loose fit between the fixation component and the device allow the fixation components to swivel within the holes of the device. The device may have spherical recesses to receive the nuts of the fixation components.

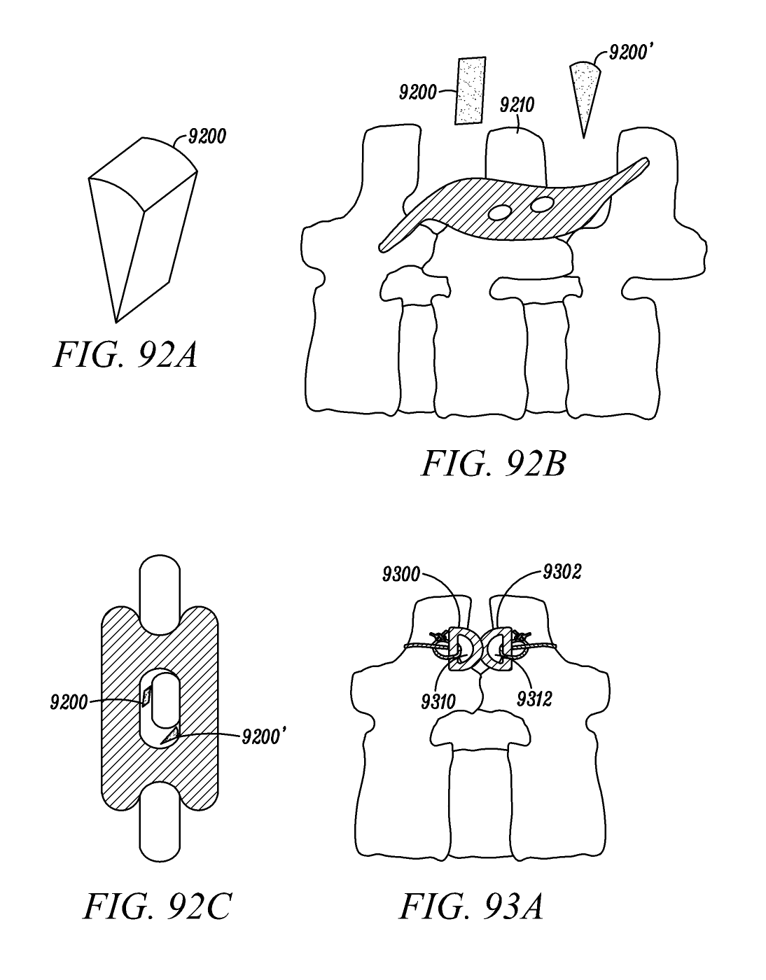

FIG. 92A is an oblique view of a shim-like device 9200 used to improve the fit between an interspinous device and the SP. The device is preferably made of bone. A portion of the device may be removed after the device is inserted between the interspinous device and the SP. The shim may also be made of metal, polymers (including PEEK), ceramic, or other material. The shims may be supplied in many different sizes and shapes.

FIG. 92B is an exploded lateral view of the spine, shims 9200, 9200′, and an alternative embodiment of the invention similar to that shown in FIG. 89A. Shims 9200, 9200′ fit between the SPS and the SP 9210. The SPS is designed to fuse to the posterior elements of the intermediate vertebra. The cranial and caudal ends of the device are sloped to fit the lamina of the cranial and caudal vertebra, respectively.

FIG. 92C is a dorsal view of the spine and the embodiment of the invention shown in FIG. 92B. A shim 9200 can be seen between the lateral aspect of the SP and the SPS and a shim 9200′ can be seen between the caudal aspect of the SP and the SPS. Bone or bone growth promoting material may be placed in the openings between the SPS, the SP and the shims. Bone or bone-growth promoting substances may also be placed in the chambers of the SPS, over the SPS, and around the posterior elements of the intermediate vertebra. The hole in the device may be customized at the time of surgery. For example, surgeons could use power burs to enlarge the hole in the device. The enlarged hole would enable surgeons to place the device over abnormally large or deformed spinous processes.

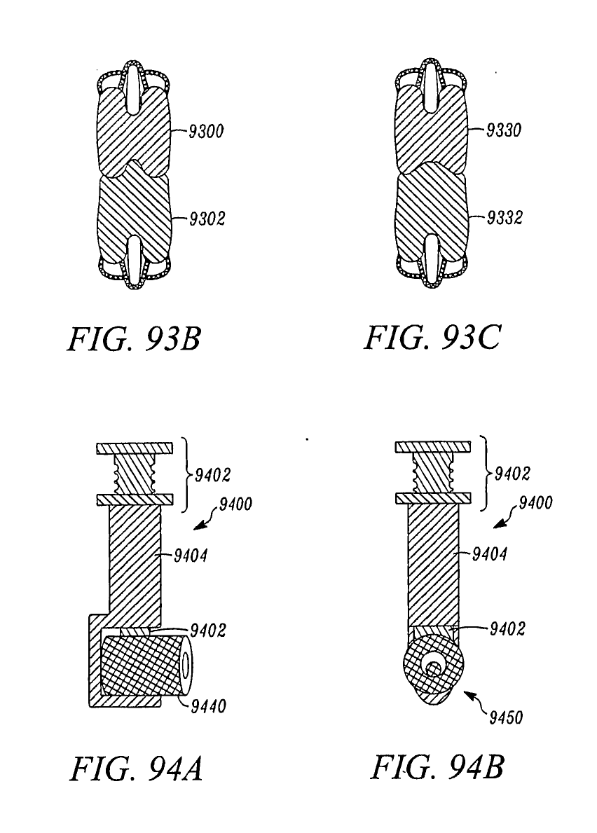

FIG. 93A is a lateral view of the spine and an alternative embodiment of the invention (see also FIG. 61). The two components 9300, 9302 of the device have chambers 9310, 9312. Bone or bone-growth promoting substances may be placed in the chambers and between each component and the SP the component partially surrounds. The cranial component 9300 is designed to fuse to the SP of the cranial vertebra. The caudal component 9302 is designed to fuse to the SP of the caudal vertebra.

FIG. 93B is a dorsal view of the spine and the embodiment of the invention shown in FIG. 93A. The components cooperate to limit spinal extension, lateral bending, and/or axial rotation. The components allow spinal flexion. The device decreases the loads across the facet joints. Decreasing the loads may decrease back pain from arthritic facet joints.

The articulating surfaces of the components may be shaped in many different ways without departing from the spirit of the invention. FIG. 93C is a dorsal view of the spine and an alternative embodiment of the invention wherein the articulating surfaces of the components 9330, 9332 are shaped differently.

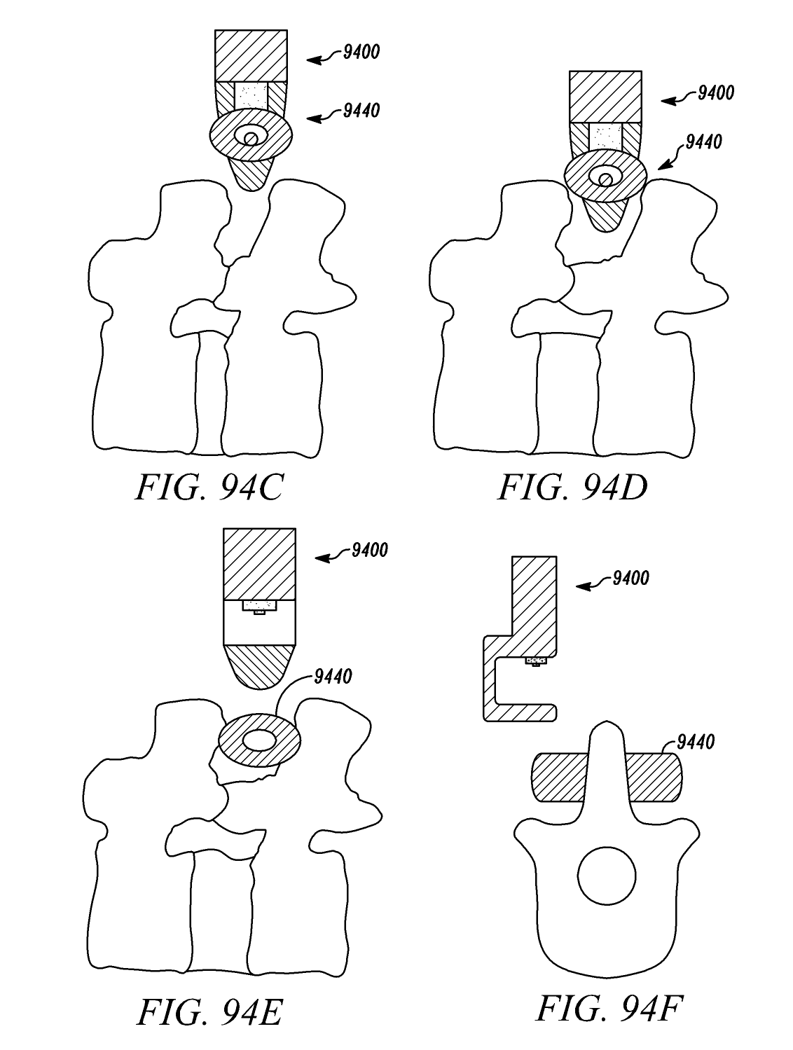

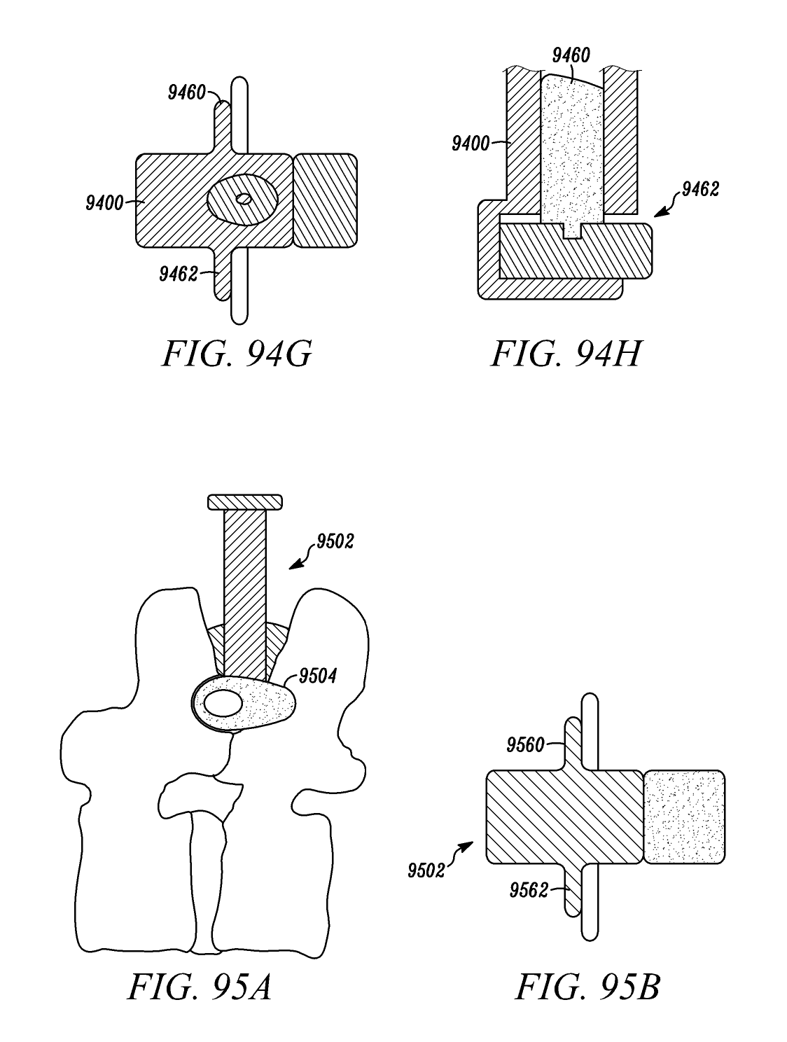

FIG. 94A is a view of the cranial side of the embodiment of the SPS shown FIG. 10A and a tool 9400 used to facilitate insertion of the SPS. The tool has two components. An impactor component 9402 passes through a cylindrical opening in a second component 9404. The SPS 9440 fits into a U-shaped opening in the side of the second component 9404. A projection (not visible) from the tip of the impactor component fits into a hole in the dorsal side of the SPS. A projection (also not visible) from the base of the U of the second component fits into a hole on the side of the SPS.

The ventral surface of the SPS lies on one of the arms of the U-shaped component. The impactor component pistons inside the second component. The impactor component is advanced into the hole of the SPS to reversibly lock the SPS in the instrument. The impactor component can be reversibly locked in the second component. For example, a spring-loaded ring could be moved from one position to the next to reversibly lock the two components. Alternatively, a nut could be advanced along the impactor component to reversibly lock the impactor component to the second component. FIG. 94B is a side view of the embodiment of the invention shown in FIG. 94A. The ventral arm 9450 of the U-shaped end of the second component is wedge-shaped in cross section.

FIG. 94C is a lateral view of the spine and the embodiment of the invention shown in FIG. 94B. FIG. 94D is a lateral view of the spine and the embodiment of the invention shown in FIG. 94C. The tool and the SPS are impacted between adjacent spinous processes. The wedge-shaped end of the tool separates the spinous processes as the tool is advanced between the spinous processes. The impactor component of the tool may be struck with a mallet to advance the tool and the SPS between the spinous processes.

FIG. 94E is an exploded lateral view of the spine and the embodiment of the invention shown in FIG. 94D. The tool has been removed from the SPS. The SPS maintains distraction of the spinous processes. Distraction of the spinous processes by the SPS enables the wedge-shaped end of the tool to be easily removed from between the spinous processes. The impactor component of the tool is withdrawn from the SPS to enable the U-shaped second component to slide off the SPS. FIG. 94F is an exploded view of the caudal end of a vertebra, a SPS, and the embodiment of the tool shown in FIG. 94E. The tool 9400 has been removed from the SPS 9440.

FIG. 94G is a dorsal view of the spine and the embodiment of the invention shown in FIG. 94F. The impactor component of the tool was not shown. Projections 9460, 9462 from the cranial and/or caudal sides of the U-shaped component of the tool fit along the sides of the Spinous processes. The projections help center the SPS between the spinous processes. Alternatively, two projections may project from both the cranial and caudal sides of the tool. The projects could straddle both sides of the spinous processes cranial and caudal to the SPS. The notch in the SPS also helps center the SPS relative to the spinous processes.

FIG. 94H is a cross section of the embodiment of the invention shown in FIG. 94A. The impactor component is depicted at 9460, and the SPS is shown at 9462. The component with the wedge component is represented at 9400.

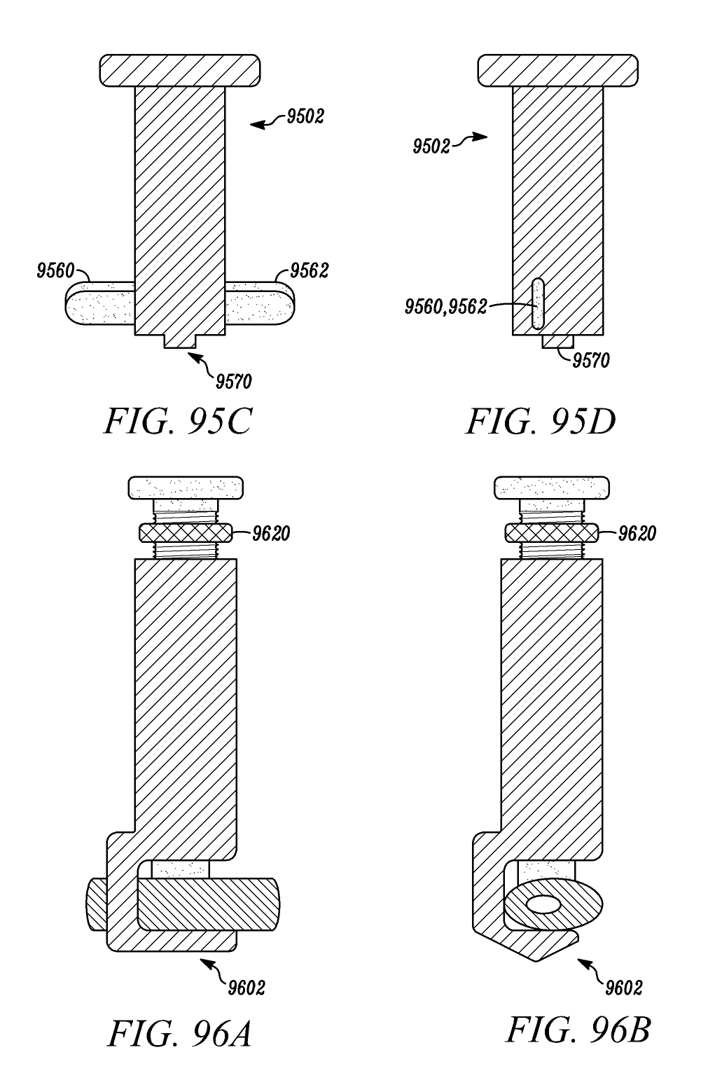

FIG. 95A is a lateral view of the spine, the embodiment of the SPS shown in FIG. 10A, and a second impactor tool 9502 used to advance the SPS 9504 towards the spinal canal. The tool may used after initial placement of the SPS by the tool shown in FIG. 94A. FIG. 95B is a dorsal view of the spine and the embodiment of the invention shown in FIG. 95A. Like the tool shown in FIG. 94G, optional projections 9560, 9562 help center the SPS in the sagittal plane of the spine. FIG. 95C is a lateral view of the tool shown in FIG. 95A. The projection 9570 from the ventral end of the tool fits into a hole in the dorsal side of the SPS. FIG. 95D is a view of the cranial side of the tool shown in FIG. 95C.

FIG. 96A is a cranial view of an alternative configuration of the invention shown in FIG. 94A, wherein the arm 9602 that connects the wedge component to the shaft of the instrument passes cranial to the SPS. Alternatively, the connecting arm may pass on the caudal side of the SPS. The piston component is threaded into the shaft of the second component. A nut 9620 may used to reversibly lock the components together. The SPS is represented by the area of the drawing with vertical and horizontal lines. FIG. 96B is a lateral view of the embodiment of the invention shown in FIG. 96B.

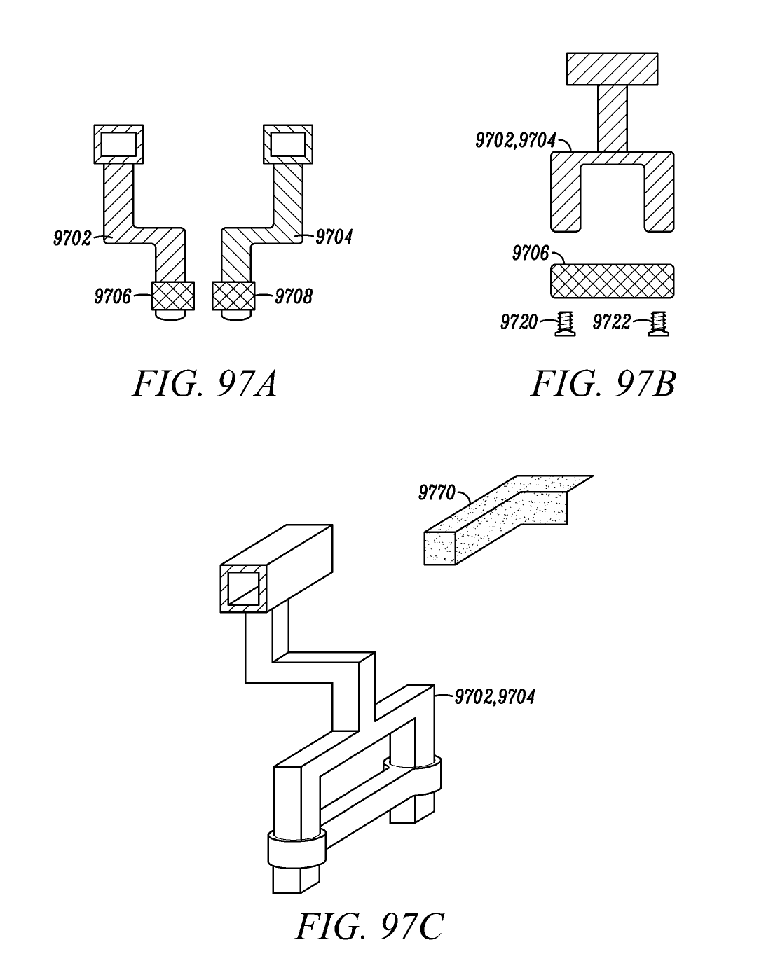

FIG. 97A is a lateral view of an alternative embodiment of the invention related to that shown in FIG. 73A. L-shaped components 9702, 9704 fit over the arms of distraction or retraction devices. For example, the L-shaped components may fit over the arms of a “McCulloch” retractor (V. Mueller Company). Flexible bands are indicated at 9706, 9708. The distraction components could be designed to fit into other instruments such as the “Caspari Distractor”.

FIG. 97B is an exploded, cranial view of the embodiment of the invention shown in FIG. 97A. The flexible band 9706 fits over the arms of the distraction component. Screws 9720, 9722 may be used to prevent the flexible band from sliding off the distraction component. Alternatively, a Velcro strap could be placed over the arms of the distraction component. FIG. 97C is an oblique view of the embodiment of the invention shown in FIG. 97A and one arm 9770 of a McCulloch retractor. The square shaped opening in the instrument fits over the square shaped arm 9770 of the retractor.

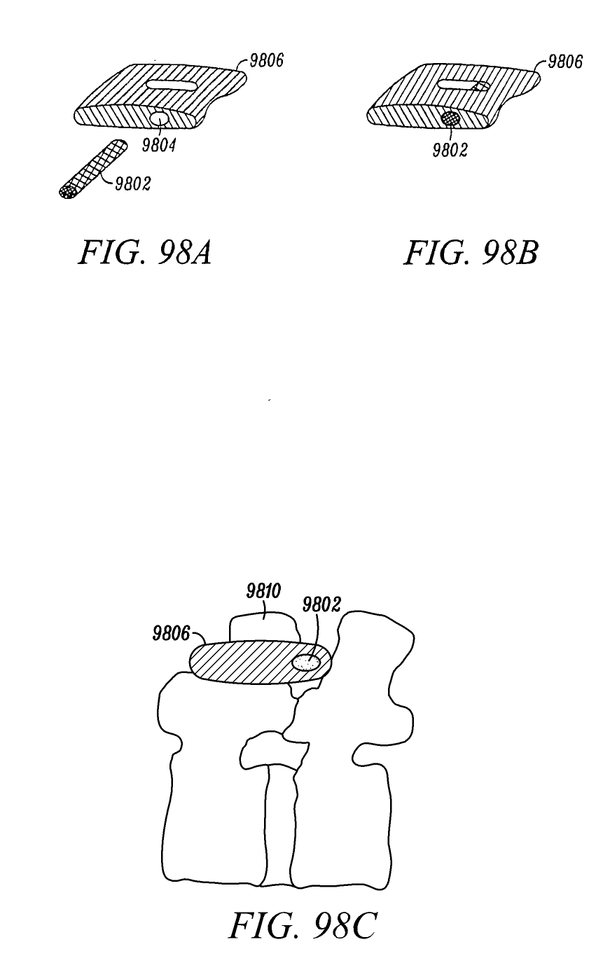

FIG. 98A is an exploded oblique view of an alternative embodiment of the invention related to that shown in FIG. 20A. A spacer component is placed over a spinous process. A dowel-like component 9802 is placed through an opening 9804 on the side of the device 9806, after the device is placed over the spinous process. The dowel component may locked into the spacer component. For example, the dowel component may be oval in cross section. Alternatively the oval dowel component could be cammed to lock the two components together.

FIG. 98B is an oblique view of an assembled device of the embodiment shown in FIG. 98A. FIG. 98C is a lateral view of the spine and the embodiment of the invention shown in FIG. 98A. The dowel component narrows the hole in the spacer component. The tip of the SP 9810 is too large to fit through the narrowed hole in the SPS. The dowel component may also increase apply pressure to the SP.

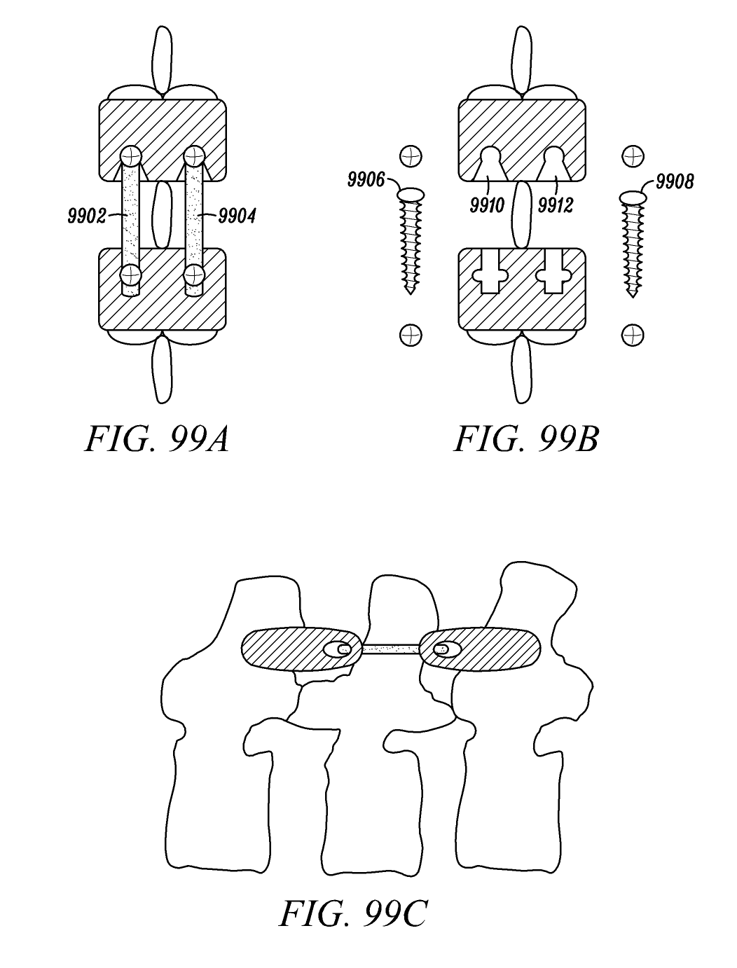

FIG. 99A is a dorsal view of the spine and an alternative embodiment of the invention including rods 9902, 9904 that connect components placed between spinous processes. One or more of the interspinous components may prevent spinal extension through the level the interspinous component was placed.

FIG. 99B is an exploded dorsal view of the embodiment of the invention drawn in FIG. 99B. The rods may have spherical enlargements 9906, 9908 on one end of the rods. The spherical enlargements of the rods articulate with spherical concavities 9910, 9912 in one of the interspinous components. Set screws hold the rods in the interspinous components. The spherical articulation between the rods and the interspinous components allow the rods to be collinear or in a non-collinear alignment. The interspinous components may be tightened over the intermediate SP.

FIG. 99C is a lateral view of the spine and the embodiment of the invention drawn in FIG. 99B. The holes in the interspinous components may be filled with bone or a bone-growth-promoting substance. The interspinous components may fuse to the posterior elements of the intermediate vertebra.

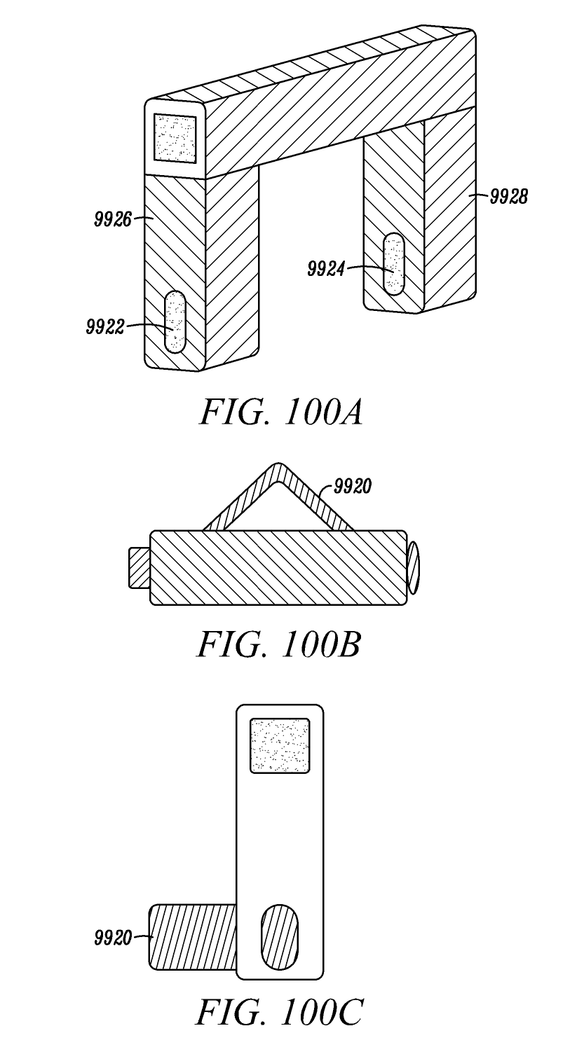

FIG. 100A is an oblique view of an alternative embodiment of the invention related to that drawn in FIG. 97A. FIG. 100B is a dorsal view of the embodiment of the invention drawn in FIG. 100A. A band 9920 has been placed through slots 9922, 9924 in the arms 9926, 9928 of the device. The band is preferably flexible. The band may be made of plastic, metal, or fibrous material. For example, a plastic cable tie could be used. The large fastening end of the cable would prevent the first end of the cable from passing completely through one arm of the device. The fastening end of a second cable tie could be affixed to the second end of the cable tie, after the second end of the cable tie is passed through the second slot in the device. The large ends cable tie trap the cable tie within the device. FIG. 100C is a lateral view of the embodiment of the device drawn in FIG. 100B.

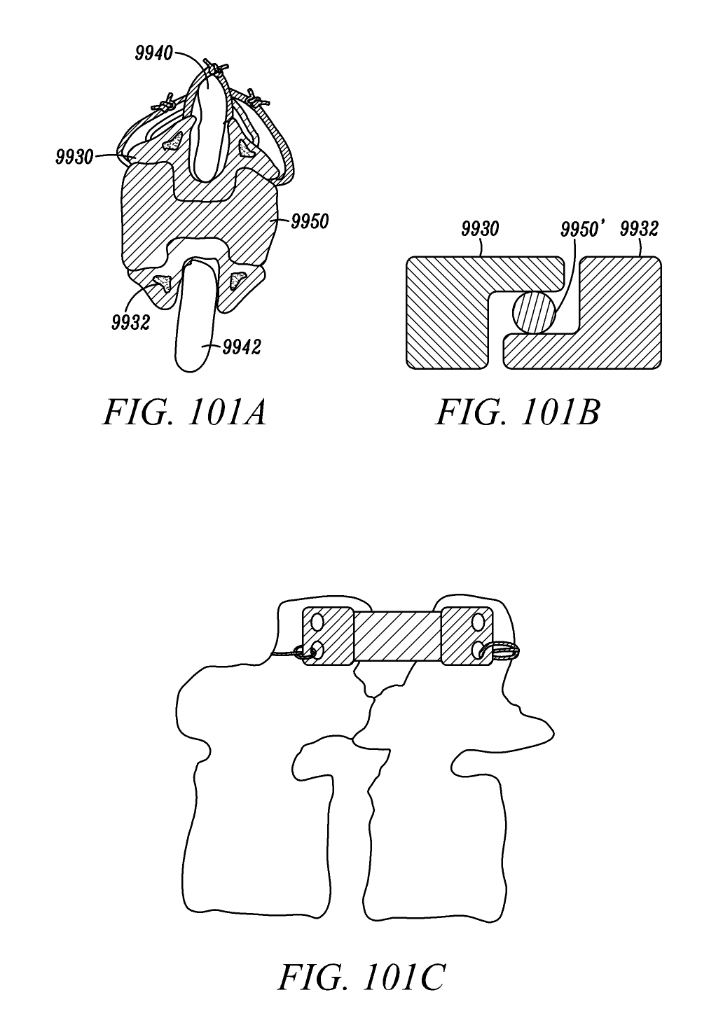

FIG. 101A is a coronal cross section of an alternative embodiment of the invention drawn in FIG. 93B. Rigid components 9930, 9932 are attached to adjacent spinous processes 9940, 9942. The fastening bands were not drawn on the component attached to the caudal SP. A flexible member 9950 is placed between the rigid components. The flexible component is preferably trapped between the rigid components without attaching to either component. The rigid components may have chambers filled with bone or bone-growth-promoting substances. The rigid components could fuse to the spinous processes. The flexible component may be made polymers, including elastomers or hydrogels. Alternatively, the intermediate component could be made of polyethylene. The polyethylene component could be attached to one of the rigid components. The large surface area of the rigid components enables transfer of loads across a larger area of the polymer component than can be transferred by the SP alone. The rigid components also surround a portion of the spinous processes. The configuration of the rigid components permits insertion of a polymer component that is larger than the space between the spinous processes. The device permits load transfer through large portions of large polymer components. The longevity of the polymer component is increased by the use of larger polymer components and by the transfer of loads through large portions of the polymer component. The polymer component could dampen the loads between the rigid components.

FIG. 101B is sagittal cross section of the embodiment of the device drawn in FIG. 101A. The areas of the drawing with closely spaced lines represent the rigid components. The area 9950′ represents a portion of the polymer component. The device is configured to allow motion between the rigid components and contain the polymer component. The polymer component could be made of more than one material or of the same material with different durometers. For example, the transverse component of the polymer component may have more tensile strength than the lateral portions of the polymer component. FIG. 101C is a lateral view of the spine and the embodiment of the device drawn in FIG. 101A.

What is claimed is:

1. A method for distracting adjacent spinous processes of a spine, comprising: inserting a device having a first side portion and a second side portion separated by a central portion, each of said first side portion and second side portion having a length that is greater than the maximum length of said central portion, wherein said length of said central portion is dimensioned to fit between said adjacent spinous processes a device having a first side portion and a second side portion separated by a central portion each of said first side portion and second side portion having a length that is greater than the maximum length of said central portion wherein said length of said central portion is dimensioned to fit between adjacent spinous processes, wherein said device is configured for insertion to an interim position between said spinous processes and wherein said central portion is situated between said adjacent spinous processes, said lengths of said first side portion, said second side portion, and said central portion are generally perpendicular to said longitudinal axis of said spine, and rotating said device from said interim position to a final position, wherein said central portion is situated between said adjacent spinous processes, and said lengths of said first side portion, said second side portion, and said central potion are generally parallel to said longitudinal axis of said spine.

2. The method of claim 1, wherein said device is configured to fuse to at least one of said adjacent spinous processes.

3. The method of claim 1, wherein said device further comprises a maximum width and wherein said maximum width is less than a minimum length of said central portion.

4. The method of claim 3, wherein said device is configured such that when positioned in said interim position said adjacent spinous processes are separated by said maximum width.

5. The method of claim 4, wherein said device is configured such that when positioned in said final position said adjacent vertebra are separated by said minimum length of said central portion.

6. The method of claim 1, wherein said device includes at least one interior cavity to permit bone growth therein.

7. The method of claim 6, wherein said cavity is configured to receive bone-growth promoting substances.

8. The method of claim 1, wherein said device is comprised of at least one of allograft and PEEK.

9. The method of claim 2, wherein said device is configured to fuse only one of said adjacent spinous processes.
